(19) United States
(12) Patent Application Publication
(10) Pub. No.: US 2026/0285930 A1
(43) Pub. Date: Sep. 24, 2026

(54) METHOD FOR PRODUCING THE SUBUNITS OF HUMAN INTERLEUKIN 35 FROM INTERLEUKIN 12 AND INTERLEUKIN 27

(71) Applicant: TECHNISCHE UNIVERSITAET MUENCHEN, IN VERTRETUNG DES FREISTAATES BAYERN, Munich (DE)

(72) Inventors: Matthias FEIGE, Munich (DE); Karen HILDENBRAND, Munich (DE)

(73) Assignee: TECHNISCHE UNIVERSITAET MUENCHEN, IN VERTRETUNG DES FREISTAATES BAYERN, Munich (DE)

(21) Appl. No.: 19/474,913

(22) PCT Filed: Apr. 12, 2024
(86) PCT No.: PCT/EP2024/059944

§ 371 Date: Oct. 13, 2025

(30) Foreign Application Priority Data
Apr. 14, 2023 (EP) 23167997.8

(51) Int. Cl. C07K 14/54; A61K 38/00; A61P 11/06; A61P 37/06; B01D 15/34; C12N 15/85

(52) U.S. Cl. CPC C07K 14/54; A61P 11/06; A61P 37/06; B01D 15/34; C12N 15/85; A61K 38/00; C07K 2319/21

(57) ABSTRACT

The present invention refers to a method for producing the α-subunit of human Interleukin 35 (SEQ ID NO: 1) from human Interleukin 12 and/or the β-subunit EBI3 of human Interleukin 35 (SEQ ID NO: 2) from human Interleukin 27. The present invention further relates to the α-subunit of human Interleukin 12 (SEQ ID NO: 3) and to the β-subunit of human Interleukin 27 (SEQ ID NO: 6) obtained according to said method of the present invention. Further, the present invention provides the nucleic acid molecule(s) comprising a nucleotide sequence enclosing said subunit(s), said nucleic acid molecule(s) being comprised in a vector, a host cell containing said nucleic acid molecule(s), and an immune modulator containing (a) nucleic acid molecule(s) according to the present invention.

### FIELD OF THE INVENTION

The present invention refers to a method for producing the α-subunit of human Interleukin 35 (SEQ ID NO: 1) from human Interleukin 12 and/or the β-subunit EBI3 of human Interleukin 35 (SEQ ID NO: 2) from human Interleukin 27. The present invention further relates to the α-subunit of human Interleukin 12 (SEQ ID NO: 3) and to the β-subunit of human Interleukin 27 (SEQ ID NO: 6) obtained according to said method of the present invention. Further, the present invention provides the nucleic acid molecule(s) comprising a nucleotide sequence enclosing said subunit(s), said nucleic acid molecule(s) being comprised in a vector, a host cell containing said nucleic acid molecule(s), an immune modulator containing (a) nucleic acid molecule(s) according to the present invention. The invention further relates to the use of any of said subunit(s) according to the present invention for the manufacture of a medicament for treating a disease in a mammal. The present invention further provides any of said subunit(s) according to the present invention for use as a medicament or a drug or for use in the treatment of a disease in a mammal. Further, the present invention provides a method of treating an Interleukin 35-mediated disease, comprising the step of administering a composition comprising the α-subunit and/or the β-subunit of human Interleukin 35 (SEQ ID NOs: 1 and 2). The present invention also relates to the use of the subunit(s) according to the present invention for producing (a) binding reagent(s) against any of said subunit(s). In a further aspect, the present invention is directed to a method for producing (a) binding reagent(s) against the α-subunit of human Interleukin 35 and/or the β-subunit of human Interleukin 35 (SEQ ID NOs: 1 and 2). The present invention also relates to a binding reagent which specifically binds to the α-subunit of human Interleukin 35 (SEQ ID NO: 1) and/or the β-subunit of human Interleukin 35 (SEQ ID NO: 2).

### BACKGROUND OF THE INVENTION

Interleukins are key signaling molecules of the immune system that are classified into families based on structural similarities (1). The Interleukin (IL) 12 family consists of at least four members (IL-12, IL-23, IL-27, and IL-35) and is assigned to one family due to its unique heterodimeric character that separates it from other ILs (2). Each member is composed of an α-subunit that shows a cytokine-characteristic four-helix bundle fold (IL-12α, IL-23α, IL-27α) and a β-subunit composed of two fibronectin (Fn) III domains (EBI3) with an optional immunoglobulin (Ig) domain (IL-12β) (2). A remarkable feature of the IL-12 family is that nature uses extensive sharing of only five subunits to build the four heterodimers (IL-12: IL-12α/IL-12β, IL-23: IL-23α/IL-12β, IL-27: IL-27α/EBI3, IL-35: IL-12α/EBI3). Likewise, this combinatorial complexity applies to the IL-12 family receptors, which are also heterodimers formed by five different chains (IL-12Rβ1, IL-12Rβ2, IL-23R, IL-27Rα, gp130). Binding of the suitable interleukin induces receptor chain dimerization, thereby activating Jak STAT signaling pathways (2).

Subunit sharing on the level of cytokines and receptors may suggest closely related functions of the IL-12 family members, but the opposite holds true. The effects of the four family members are surprisingly diverse and even opposing. The mostly pro-inflammatory IL-12 and IL-23 are drivers of inflammation via Th1 differentiation and Th17 development respectively (3). IL-27 is an immunomodulatory cytokine, which on the one hand is able to promote Th1 differentiation and on the other hand suppresses pro-inflammatory Th17 cells and induces anti-inflammatory IL-10 producing T regulatory (Tr) 1 cells (4). In contrast, IL-35 is the only strictly inhibitory family member and acts by suppression of conventional T cells and their conversion into induced suppressive iTr35 cells (5).

Among the IL-12 family cytokines, IL-35 remains particularly unintelligible, despite its many important biological functions. In contrast to the other family members, IL-35 is predominantly produced by subsets of regulatory T and B cells, to a lesser extent by dendritic cells (DCs), but also placental trophoblasts and tumor cells, and is mainly involved in the immunosuppressive capabilities of these different cell types (5). Due to its inhibitory character, IL-35 plays an important role in keeping immune reactions in check, but is also associated with a broad range of different immune-related diseases. On the one hand, a variety of autoimmune, inflammatory, and allergic diseases are linked to reduced circulating IL-35 levels, underlining its importance in maintaining self-tolerance (6-9). On the other hand, IL-35 is able to induce tumor growth by enhancing myeloid cell accumulation and promoting angiogenesis (10). High serum levels can be found in various cancers including breast, pancreatic, and lung cancer (11-16). In strong contrast to the detailed insights into its many biological functions stands the understanding of the structure and receptor repertoire of IL-35. For IL-12, IL-23 and IL-27, experimental structures and experimentally validated models of the isolated cytokines (17-20) and their receptor binding are available (21-23). In case of IL-35, even comprehensive studies have failed to identify its heterodimerization interface (24), which thus is clearly distinct from its other family members. Furthermore, recent studies point towards independent functions of IL-35 subunits, but insights into the biological relevance and molecular mechanisms remain very limited (25-28). Lastly, IL-35 signaling does not occur via a single heterodimeric receptor, as described for the other members, but four possible receptor chain combinations (IL-12Rβ2:gp130, IL-12Rβ2:IL-12Rβ2, gp130:gp130, IL-12Rβ2:IL-27Rα) are so far reported as IL-35 receptors (28, 29). This pronounced discrepancy between the biological importance and our molecular understanding of IL-35 demands for further studies, in particular since the biological functions of IL-35 qualify it as a molecule of potentially very high medical relevance.

Concerning the present invention, the inventors have found that IL-35 subunits mutually promote their secretion from cells not only as a heterodimer, but also as non-heterodimers. Thus, the inventors of the present invention performed the first detailed biochemical analysis of the IL-35 human subunits IL-12α and EBI3 and show that these can independently act as anti-inflammatory cytokines. Additionally, the method according to the present invention is the first method which allows the production of the subunits of IL-35 in high yields, which was not possible before the present invention. The method allows to receive the respective subunit(s) of IL-35 in milligram-amounts per 100 ml cell-culture and due to distinct steps of the present invention, the inventors were able to provide homogenous and active subunits of IL-35.

### SUMMARY OF THE INVENTION

In a first aspect, the present invention provides a method for producing the α-subunit of human Interleukin 35 (SEQ ID NO: 1) from human Interleukin 12 and/or the β-subunit EBI3 of human Interleukin 35 (SEQ ID NO: 2) from human Interleukin 27, comprising the steps of:
(a) introducing into a nucleic acid molecule encoding the recombinant α- and β-subunits of the human Interleukin 12 polypeptide (SEQ ID NOs: 3 and 4) a nucleotide sequence mutating at least one amino acid residue of the α-subunit of human Interleukin 12 and/or mutating at least one amino acid residue of the β-subunit of human Interleukin 12, and/or
(b) introducing into a nucleic acid molecule encoding the recombinant α- and β-subunits of the human Interleukin 27 polypeptide (SEQ ID NOs: 5 and 6) a nucleotide sequence mutating at least one amino acid residue of the α-subunit of human Interleukin 27, and
(c) introducing the obtained nucleic acid molecule(s) of steps (a) and/or (b) for expression into a suitable host cell or into a suitable cell extract or cell lysate.

In a further aspect, the present invention provides the α-subunit of human Interleukin 12 (SEQ ID NO: 3) obtained according to the method of the present invention.

In a further aspect, the present invention provides the β-subunit of human Interleukin 27 (SEQ ID NO: 6) obtained according to the method of the present invention.

The present invention also provides the nucleic acid molecule comprising a nucleotide sequence encoding the α-subunit of human Interleukin 12 (SEQ ID NO: 3) according to the present invention. The present invention also relates to the nucleic acid molecule comprising a nucleotide sequence encoding the β-subunit of human Interleukin 27 (SEQ ID NO: 6) according to the present invention.

In a further aspect, the present invention provides a host cell containing a nucleic acid molecule according to the present invention.

The present invention also provides an immune modulator comprising a subunit according to the present invention.

In a further aspect, the present invention provides the use of the α-subunit of human Interleukin 35 (SEQ ID NO: 1) and/or of the β-subunit of human Interleukin 35 (SEQ ID NO: 2), preferably (a) subunit(s) obtained according to the method of the present invention, for the manufacture of a medicament for treating a disease in a mammal.

The present invention also provides the α-subunit of human Interleukin 35 (SEQ ID NO: 1) and/or the β-subunit of human Interleukin 35 (SEQ ID NO: 2), preferably (a) subunit(s) obtained according to the method of the present invention, for use as a medicament.

Additionally, the present invention provides the α-subunit of human Interleukin 35 (SEQ ID NO: 1) and/or the β-subunit of human Interleukin 35 (SEQ ID NO: 2), preferably (a) subunit(s) obtained according to the method of the present invention, for use as a drug.

The present invention also provides the α-subunit of human Interleukin 35 (SEQ ID NO: 1) and/or the β-subunit of human Interleukin 35 (SEQ ID NO: 2), preferably (a) subunit(s) obtained according to the method of the present invention, for use in the treatment of a disease in a mammal.

Additionally, the present invention provides a method of treating an Interleukin 35-mediated disease, comprising the step of administering a composition comprising the α-subunit and/or the β-subunit of human Interleukin 35 (SEQ ID NOs: 1 and 2) according to the present invention.

The present invention also provides the use of the α-subunit of human Interleukin 35 (SEQ ID NO: 1) and/or the β-subunit of human Interleukin 35 (SEQ ID NO: 2), for producing (a) binding reagent(s) against any of said subunit(s).

The present invention also provides a method for producing (a) binding reagent(s) against the α-subunit of human Interleukin 35 (SEQ ID NO: 1) and/or the β-subunit of human Interleukin 35 (SEQ ID NO: 2).

The present invention further provides a binding reagent which specifically binds to the α-subunit of human Interleukin 35 (SEQ ID NO: 1) and/or the β-subunit of human Interleukin 35 (SEQ ID NO: 2).

These aspects of the invention will be more fully understood in view of the following drawings, detailed description and non-limiting examples.

### BRIEF DESCRIPTION OF THE DRAWINGS

The accompanying drawings are included to further an understanding of the embodiments that are incorporated in and constitute a part of this specification. The drawings illustrate embodiments and together with the description serve to explain principles of embodiments. Other embodiments and many of the intended advantages of embodiments will be readily appreciated, as they become better understood by reference to the detailed description. The elements of the drawings are not necessarily to scale relative to each other.

FIG. 1 shows that IL-12α (SEQ ID NO: 3) and EBI3 (SEQ ID NO: 6) mutually promote their secretion and form IL-35. FIG. 1a shows that IL-35 (alphaFold2 docked model) shares its α-subunit IL-12α (SEQ ID NO: 3) with IL-12 (PDB: 3HMX), and its β-subunit EBI3 (SEQ ID NO: 6) with IL-27 (PDB: 7u7n). To assess mutually induced secretion of IL-12α (SEQ ID NO: 3) and EBI3 (SEQ ID NO: 6), constant DNA amounts of IL-12α (SEQ ID NO: 3) were co-transfected with increasing DNA amounts of EBI3 (SEQ ID NO: 6) (FIG. 1b), or vice versa (FIG. 1c). Both, IL-12α (SEQ ID NO: 3) and EBI3 (SEQ ID NO: 6) show a reduced mobility in the medium, indicating that both subunits are retained in the endoplasmic reticulum in isolation and traverse the Golgi during secretion, as indicated by modification of their glycans. FIG. 1d shows co-immunoprecipitation of FLAG-tagged IL-12α (SEQ ID NO: 3) co-expressed with EBI3 (SEQ ID NO: 6) in the cell medium verifies assembly for these two proteins. Quantification of the IL-12α (SEQ ID NO: 3) IP efficiency and the fraction of EBI3 (SEQ ID NO: 6), which is found in complex with IL-12α (SEQ ID NO: 3), is shown (n=3±SD). FIG. 1e shows co-immunoprecipitation of secreted HA-tagged EBI3 (SEQ ID NO: 6) co-expressed with IL-12α^{FLAG} verifies assembly for these two proteins in the medium. Quantification of the EBI3-HA IP efficiency and the fraction of IL-12α^{FLAG}, which is found in complex with EBI3-HA is shown (n=5±SD). Constructs were expressed in HEK293T cells. One representative immunoblot is shown in each case.

FIG. 2 shows that IL-35 subunits (SEQ ID NOs: 1 and 2) can be secreted as non-heterodimers in contrast to IL-12 and IL-27. FIG. 2a shows that EBI3 (SEQ ID NO: 6) induces the secretion of IL-12α (SEQ ID NO: 3), even when it is retained in the ER (via a C-terminal KDEL sequence). EBI3-induced secretion is observed for wild-type IL-12α (SEQ ID NO: 3) and a variant lacking the cysteine that forms an interchain disulfide bond in IL-12α (C96S, SEQ ID NO: 7). FIG. 2b shows the same as in FIG. 2a, only that IL-12α (SEQ ID NO: 3) was furnished with a KDEL ER-retention sequence and secretion of EBI3 (SEQ ID NO: 6) was monitored. EBI3 (SEQ ID NO: 6) secretion was slightly increased by co-expression with both IL-12α (SEQ ID NO: 3) and the C96S variant (SEQ ID NO: 7). Quantifications of EBI3 (SEQ ID NO: 6) secretion are shown below the blot. FIG. 2c shows a similar analysis for IL-12, which reveals that IL-12β^{C199S} (SEQ ID NO: 8), with an ER retention sequence, does not induce secretion of free IL-12α (SEQ ID NO: 3), but instead co-retains it in the cell. The same is observed for the combination of IL-12α^{C96S} (SEQ ID NO: 7) with IL-12β^{C199S} (SEQ ID NO: 8), with both proteins lacking the cysteines that form the interchain disulfide bond in IL-12. FIG. 2d shows that co-expression of wildtype EBI3 (SEQ ID NO: 6) and IL-27a (SEQ ID NO: 5) leads to the secretion of IL-27. When IL-27a (SEQ ID NO: 5) was ER-retained (IL-27α^{KDEL}, SEQ ID NO: 31), EBI3 (SEQ ID NO: 6) secretion was reduced, in contrast to FIG. 2b. FIG. 2e shows that free IL-12α (SEQ ID NO: 3) can be detected in medium samples after pulldown of EBI3 (SEQ ID NO: 6). IL-12α (SEQ ID NO: 3) and EBI3 (SEQ ID NO: 6) are co-transfected and cell supernatants underwent two consecutive HA-IPs to isolate EBI3^{HA} containing complexes. IL-12α (SEQ ID NO: 3) is also co-immunoprecipitated as can be seen in the IL-12α (SEQ ID NO: 3) blot after HA-IP I and II indicating pulldown of IL-35. The final IL-12α-IP reveals remaining IL-12α (SEQ ID NO: 3) in the medium that is not interacting with EBI3 (SEQ ID NO: 6). Band intensities show a significantly higher amount of free IL-12α (SEQ ID NO: 3) when co-expressed with EBI3 (SEQ ID NO: 6) (or EBI3^{KDEL}, SEQ ID NO: 29) compared to transfection in isolation without EBI3 (SEQ ID NO: 6).

FIG. 3 shows that recombinant human IL-12α^{C96S} (SEQ ID NO: 7) and EBI3 (SEQ ID NO: 6) are stable and well-structured proteins. FIG. 3a shows the analysis of purified IL-12α^{C96S} (SEQ ID NO: 5) and EBI3 (SEQ ID NO: 6) by reducing and non-reducing SDS-PAGE. Faster migration on non-reducing SDS-PAGE indicates the presence of disulfide bonds. Positions of intramolecular disulfide bonds are indicated in each subunit structure. FIG. 3b shows that reconstituted IL-12 and IL-27 are able to induce receptor heterodimerization. COS-7 cells were co-transfected with the indicated receptor chains equipped with the NanoBRET reporter system. Cells were stimulated with purified IL-12α^{C96S} (SEQ ID NO: 7), which was previously incubated with recombinant human IL-12β^{C199S} (SEQ ID NO: 8), or EBI3 (SEQ ID NO: 6), previously incubated with murine IL-27α^{His} (SEQ ID NO: 33) or the isolated subunits as indicated (10 nM final concentrations). Graphs represent the normalized NanoBRET signal (n=3±SD). Statistical significance was calculated by one way ANOVA followed by Dunnett's multiple comparison test; ****p<0.0001 compared with the corresponding PBS control. FIG. 3c shows that NK-92 or BL-2 cells were stimulated with pre-incubated IL-12α^{C96S} (SEQ ID NO: 7)+IL-12β^{C199S} (SEQ ID NO: 8) or EBI3 (SEQ ID NO: 6)+mIL-27α^{His} (SEQ ID NO: 33) and the heterodimeric cytokines (10 ng/ml final concentrations). Downstream signaling was detected by STAT-phosphorylation via immunoblot. FIG. 3d shows far-UV CD spectra for IL-12α^{C96S} (SEQ ID NO: 7) and EBI3 (SEQ ID NO: 6). FIG. 3e shows that IL-12α^{C96S} (SEQ ID NO: 7) unfolds cooperatively with an apparent melting temperature of 47±0.2° C. and EBI3 (SEQ ID NO: 6) with an apparent melting temperature of 50±0.2° C. (green line: experimental data; black line: Boltzmann sigmoidal non-linear curve fit; transitions were not reversible).

FIG. 4 shows that IL-12α^{C96S} (SEQ ID NO: 7) and EBI3 (SEQ ID NO: 6) act as immunosuppressors. FIG. 4a shows concentrations of secreted IL-1β, IL-6, IL-8 and TNFα (ELISA) in supernatants from LPS stimulated human PBMCs after cytokine stimulation (n=10 donors). FIG. 4b shows a scheme of the experimental workflow to assess IL-12α^{C96S} (SEQ ID NO: 7) and EBI3 (SEQ ID NO: 6) effects on alveolar like monocyte-derived macrophages (MDM). FIG. 4c shows amounts of IL-1β, IL-6, IL-8, and TNFα (ELISA) produced by human MDM (n=10 to 17 donors) in supernatants after stimulation with HDM and cytokines. In each case, 10 ng/ml cytokine was used. Data are presented as means+SEM. Statistical significance was determined by Friedmann test. *p<0.05; **p<0.01.

FIG. 5 shows that IL-12α^{C96S} (SEQ ID NO: 7) and EBI3 (SEQ ID NO: 6) do not inhibit IL-12 or IL-27 signaling. FIG. 5a shows that the NanoBRET reporter system indicates IL-12 receptor heterodimerization after stimulation with 10 nM IL-12, which is not blocked by a 30 min pre-treatment with IL-12α^{C69S} (SEQ ID NO: 7) (10, 100 or 1000 nM). FIG. 5b shows that the same is observed for IL-27 and IL-27 receptor heterodimerization upon EBI3 pre-treatment. Graphs represent the normalized NanoBRET signal (n=3±SD). FIG. 5c shows that STAT4 phosphorylation in NK-92 cells after treatment with 10 ng/ml IL-12 is not blocked by a 30 min pre-treatment with IL-12α^{C69S} (SEQ ID NO: 7) (10, 100 or 1000 ng/ml; n=3±SD). FIG. 5d shows that BL-2 cells were used to monitor IL-27 (10 ng/ml) mediated STAT1 phosphorylation, which is not blocked by pre-treatment with EBI3 (SEQ ID NO:6) (10, 100 or 1000 ng/ml) for 30 min (n=3±SD). FIGS. 5e and 5f show that NK-92 (FIG. 5e) respectively BL-2 cells (FIG. 5f) were treated with 10 ng/ml IL-12α^{C69S} (SEQ ID NO: 7) or EBI3 (SEQ ID NO: 6) o/n to investigate receptor internalization. No inhibiting effect on STAT4 or STAT1 phosphorylation is observed after stimulation with IL-12 or IL-27 (both 10 ng/ml), instead EBI3 (SEQ ID NO: 6) treatment o/n leads to a weak induction of STAT1 phosphorylation, which is additive to IL-27-induced STAT1 phosphorylation (n=3±SD, *p<0.05; ****p<0.0001).

FIG. 6 shows that IL-12α^{C69S} (SEQ ID NO: 7) and EBI3 (SEQ ID NO: 6) potentiate regulatory T cell development and suppress SEA-induced IL-4 production from human PBMCs. FIG. 6a shows the percentage of CD25⁺Foxp3⁺ Treg cells (FACS) in human PBMC cultures treated with IL-12α^{C69S} (SEQ ID NO: 7), EBI3 (SEQ ID NO: 6) (10 ng/ml) or PBS (control) (n=8). FIG. 6b shows the concentrations of IL-4 (ELISA) in culture supernatants from PBS (ctrl) and SEA (50 μg/ml)-stimulated human PBMCs alone or in combination with IL-12α^{C96S} (SEQ ID NO: 7) or EBI3 (SEQ ID NO: 6) (10 ng/ml) (n=8 donors). Dashed lines show the percentage of Treg cells or IL-4 levels in presence of PBS control. Data are presented as means+SEM. Statistical significance was determined by Friedmann test. *p<0.05; **p<0.01.

FIG. 7 shows that co-immunoprecipitation of HA-tagged EBI3 (SEQ ID NO: 6) with FLAG-tagged IL-12α (SEQ ID NO: 3) in cell lysates verifies assembly for these two proteins. Constructs were expressed in HEK293T cells. One representative immunoblot is shown.

FIG. 8 shows that recombinant IL-12α^{C96S} (SEQ ID NO: 7) and EBI3 (SEQ ID NO: 6) are pure and interaction-competent proteins. FIGS. 8a and 8b show the detailed purification strategy with the corresponding Coomassie stained SDS-gels of IL-12α^{C96S} (SEQ ID NO: 7) (FIG. 8a) and EBI3 (SEQ ID NO: 6) (FIG. 8b). Pooled fractions are shown in rectangles. For EBI3 (SEQ ID NO: 6), the red dashed line is shown to guide the eye for the comparison of its migration behavior under reducing versus non-reducing conditions. FIG. 8c shows that recombinant IL-12α^{C96S} (SEQ ID NO: 7) and EBI3 (SEQ ID NO: 6) interact with their partner subunits to form IL-12 and IL-27, respectively. IL-12α^{C69S} (SEQ ID NO: 7) and IL-12β^{C199S,His} (SEQ ID NO: 35) or EBI3 (SEQ ID NO: 6) and murine IL-27α^{His} (SEQ ID NO: 33) were incubated and co-immunoprecipitated using the His-tag, which reveals specific interaction. Murine IL-27α^{His} (SEQ ID NO: 33) was used for all experiments and was needed since human IL-27a (SEQ ID NO: 5) cannot be produced in isolation⁴³.

FIG. 9 shows that recombinant IL-12α^{C69S} (SEQ ID NO: 7) and EBI3 (SEQ ID NO: 6) do not stably interact to form IL-35. FIG. 9a shows analytical ultracentrifugation experiments of IL-12α^{C69S} (SEQ ID NO: 7) and EBI3 (SEQ ID NO: 6) confirm a mostly monomeric state of both proteins, with frictional ratios of 1.31 (IL-12α^{C69S}, SEQ ID NO: 7) and 1.45 (EBI3, SEQ ID NO: 6) and calculated molecular weights for the monomers of 27.3 kDa (IL-12α^{C69S}, SEQ ID NO: 7) and 27.5 kDa (EBI3, SEQ ID NO: 6). Incubation of both proteins did not indicate the formation of a heterodimeric complex. FIG. 9b shows that individually purified IL-12α^{C96S,His} (SEQ ID NO: 32) and EBI3 (SEQ ID NO: 6) do not co-immunoprecipitate in contrast to EBI3 (SEQ ID NO: 6) and mIL-27α^{His} (SEQ ID NO: 33). FIG. 9c shows that hydrogen/deuterium exchange (HDX) experiments reveal IL-12α^{C96S} (SEQ ID NO: 7) stabilization through complex formation only after incubation with IL-12β^{C199S} (SEQ ID NO: 8) in contrast to EBI3 (SEQ ID NO: 6). IL-12α^{C96S} (SEQ ID NO: 7) is colored according to the fractional uptake of HDX measurements. Blue color indicates a low (less flexible and potentially shielded regions) and red colors a high (flexible and solvent accessible regions) fractional uptake (gray: no sequence coverage in HDX measurements).

FIG. 10 shows gene expression of IL1B, IL6, CXCL8, TNFA, TGFB and IL5 (qPCR) from LPS-stimulated human PBMCs after additional cytokine treatment (n=6 to 10 donors). Data are presented as means+SEM. Statistical significance was determined by Friedmann test (*p<0.05; **p<0.01).

FIG. 11 shows gene expression of IL5 (qPCR) from LPS stimulated human PBMCs after additional cytokine treatment (n=7 donors). Data are presented as means+SEM. Statistical significance was determined by Friedmann test. *p<0.05.

FIG. 12 shows that CD81 does not co-immunoprecipitate with IL-12α (SEQ ID NO: 3) and EBI3 (SEQ ID NO: 6). Co-immunoprecipitation of FLAG-tagged IL-12α (SEQ ID NO: 3) with HA-tagged EBI3 (SEQ ID NO: 6) in cell lysates and in the medium verifies assembly for these two proteins. Endogenous CD81 can be detected in the input lysate fraction only.

### DETAILED DESCRIPTION OF THE INVENTION

The following language and descriptions of certain preferred embodiments of the present invention are provided in order to further an understanding of the principles of the present invention. However, it will be understood that no limitations of the present invention are intended, and that further alterations, modifications, and applications of the principles of the present invention are also included.

In general, the present invention is directed to a method for producing the α-subunit of human Interleukin 35 (SEQ ID NO: 1) from human Interleukin 12 and/or the β-subunit EBI3 of human Interleukin 35 (SEQ ID NO: 2) from human Interleukin 27, comprising the steps of:
(a) introducing into a nucleic acid molecule encoding the recombinant α- and β-subunits of the human Interleukin 12 polypeptide (SEQ ID NOs: 3 and 4) a nucleotide sequence mutating at least one amino acid residue of the α-subunit of human Interleukin 12 (SEQ ID NO: 3) and/or mutating at least one amino acid residue of the β-subunit of human Interleukin 12 (SEQ ID NO: 4), and/or
(b) introducing into a nucleic acid molecule encoding the recombinant α- and β-subunits of the human Interleukin 27 polypeptide (SEQ ID NOs: 5 and 6) a nucleotide sequence mutating at least one amino acid residue of the α-subunit of human Interleukin 27 (SEQ ID NO: 5), and
(c) introducing the obtained nucleic acid molecule(s) of steps (a) and/or (b) for expression into a suitable host cell or into a suitable cell extract or cell lysate.

In a further aspect, the present invention is also directed to a method for producing the α-subunit of human Interleukin 35 (SEQ ID NO: 1) from human Interleukin 12 and/or the β-subunit EBI3 of human Interleukin 35 (SEQ ID NO: 2) from human Interleukin 27, comprising the steps of:
(a) introducing into a nucleic acid molecule encoding the recombinant α- and β-subunits of the human Interleukin 12 polypeptide (SEQ ID NOs: 3 and 4) a nucleotide sequence mutating at least one amino acid residue of the α-subunit of human Interleukin 12 (SEQ ID NO: 3) and having at least 70%, at least 75%, at least 80%, at least 85%, at least 90%, at least 91%, at least 92%, at least 93%, at least 94%, at least 95%, at least 96%, at least 97%, at least 98% or at least 99% sequence identity to the α-subunit of human Interleukin 12 (SEQ ID NO: 3) and/or a nucleotide sequence mutating at least one amino acid residue of the β-subunit of human Interleukin 12 (SEQ ID NO: 4) and having at least 70%, at least 75%, at least 80%, at least 85%, at least 90%, at least 91%, at least 92%, at least 93%, at least 94%, at least 95%, at least 96%, at least 97%, at least 98% or at least 99% sequence identity to the β-subunit of human Interleukin 12 (SEQ ID NO: 4), and/or
(b) introducing into a nucleic acid molecule encoding the recombinant α- and β-subunits of the human Interleukin 27 polypeptide (SEQ ID NOs: 5 and 6) a nucleotide sequence mutating at least one amino acid residue of the α-subunit of human Interleukin 27 (SEQ ID NO: 5) and having at least 70%, at least 75%, at least 80%, at least 85%, at least 90%, at least 91%, at least 92%, at least 93%, at least 94%, at least 95%, at least 96%, at least 97%, at least 98% or at least 99% sequence identity to the α-subunit of human Interleukin 27 (SEQ ID NO: 5), and
(c) introducing the obtained nucleic acid molecule(s) of steps (a) and/or (b) for expression into a suitable host cell or into a suitable cell extract or cell lysate.

By “identity” or “sequence identity” is meant a property of sequences that measures their similarity or relationship. The term “sequence identity” or “identity” as used in the present invention means the percentage of pair-wise identical residues—following (homology) alignment of a sequence of a polypeptide of the invention with a sequence in question—with respect to the number of residues in the longer of these two sequences. Identity is measured by dividing the number of identical residues by the total number of residues and multiplying the product by 100.

The percentage of sequence homology or sequence identity can, for example, be determined herein using the program BLASTP, version blastp 2.2.5 (Nov. 16, 2002; cf. Altschul, S. F. et al. (1997) Nucl. Acids Res. 25, 3389-3402). In this embodiment, the percentage of homology is based on the alignment of the entire polypeptide sequences (matrix: BLOSUM 62; gap costs: 11.1; cutoff value set to 10⁻³) including the respective sequences. It is calculated as the percentage of numbers of “positives” (homologous amino acids) indicated as result in the BLASTP program output divided by the total number of amino acids selected by the program for the alignment.

In a further aspect, the present invention is directed to a method for producing the α-subunit of human Interleukin 35 (SEQ ID NO: 1) from human Interleukin 12 and/or the β-subunit EBI3 of human Interleukin 35 (SEQ ID NO: 2) from human Interleukin 27, comprising the steps of:
(a) introducing into a nucleic acid molecule encoding the recombinant α- and β-subunits of the human Interleukin 12 polypeptide (SEQ ID NOs: 3 and 4) a nucleotide sequence mutating one amino acid residue of the α-subunit of human Interleukin 12 (SEQ ID NO: 3) and/or mutating one amino acid residue of the β-subunit of human Interleukin 12 (SEQ ID NO: 4), and/or
(b) introducing into a nucleic acid molecule encoding the recombinant α- and β-subunits of the human Interleukin 27 polypeptide (SEQ ID NOs: 5 and 6) a nucleotide sequence mutating one amino acid residue of the α-subunit of human Interleukin 27 (SEQ ID NO: 5), and
(c) introducing the obtained nucleic acid molecule(s) of steps (a) and/or (b) for expression into a suitable host cell or into a suitable cell extract or cell lysate.

In a preferred embodiment, the method of the present invention is directed to a method for producing the α-subunit of human Interleukin 35 (SEQ ID NO: 1) from human Interleukin 12 and/or the β-subunit EBI3 of human Interleukin 35 (SEQ ID NO: 2) from human Interleukin 27, comprising the steps of:
(a) introducing into a nucleic acid molecule encoding the recombinant α- and β-subunits of the human Interleukin 12 polypeptide (SEQ ID NOs: 3 and 4) a nucleotide sequence mutating one amino acid residue of the α-subunit of human Interleukin 12 (SEQ ID NO: 3), and/or
(b) introducing into a nucleic acid molecule encoding the recombinant α- and β-subunits of the human Interleukin 27 polypeptide (SEQ ID NO: 5 and 6) a nucleotide sequence mutating one amino acid residue of the α-subunit of human Interleukin 27 (SEQ ID NO: 5), and
(c) introducing the obtained nucleic acid molecule(s) of steps (a) and/or (b) for expression into a suitable host cell or into a suitable cell extract or cell lysate.

In one embodiment, the method of the present invention is directed to a method for producing the α-subunit of human Interleukin 35 (SEQ ID NO: 1) from human Interleukin 12 and/or the β-subunit EBI3 of human Interleukin 35 (SEQ ID NO: 2) from human Interleukin 27, comprising the steps of:
(a) introducing into a nucleic acid molecule encoding the recombinant α- and β-subunits of the human Interleukin 12 polypeptide (SEQ ID NOs: 3 and 4) a nucleotide sequence mutating one amino acid residue of the β-subunit of human Interleukin 12 (SEQ ID NO: 4), and/or
(b) introducing into a nucleic acid molecule encoding the recombinant α- and β-subunits of the human Interleukin 27 polypeptide (SEQ ID NO: 5 and 6) a nucleotide sequence mutating one amino acid residue of the α-subunit of human Interleukin 27 (SEQ ID NO: 5), and
(c) introducing the obtained nucleic acid molecule(s) of steps (a) and/or (b) for expression into a suitable host cell or into a suitable cell extract or cell lysate.

In one embodiment, the present invention is directed to a method for producing the α-subunit of human Interleukin 35 (SEQ ID NO: 1) from human Interleukin 12, comprising the steps of:
(a) introducing into a nucleic acid molecule encoding the recombinant α- and β-subunits of the human Interleukin 12 polypeptide (SEQ ID NOs: 3 and 4) a nucleotide sequence mutating at least one amino acid residue of the α-subunit of human Interleukin 12 (SEQ ID NO: 3), and
(b) introducing the obtained nucleic acid molecule of step (a) for expression into a suitable host cell or into a suitable cell extract or cell lysate.

In one embodiment, the present invention is directed to a method for producing the α-subunit of human Interleukin 35 (SEQ ID NO: 1) from human Interleukin 12, comprising the steps of:
(a) introducing into a nucleic acid molecule encoding the recombinant α- and β-subunits of the human Interleukin 12 polypeptide (SEQ ID NOs: 3 and 4) a nucleotide sequence mutating one amino acid residue of the α-subunit of human Interleukin 12 (SEQ ID NO: 3), and
(b) introducing the obtained nucleic acid molecule of step (a) for expression into a suitable host cell or into a suitable cell extract or cell lysate.

In one embodiment, the present invention is directed to a method for producing the α-subunit of human Interleukin 35 (SEQ ID NO: 1) from human Interleukin 12, comprising the steps of:
(a) introducing into a nucleic acid molecule encoding the recombinant α- and β-subunits of the human Interleukin 12 polypeptide (SEQ ID NOs: 3 and 4) a nucleotide sequence mutating at least one amino acid residue of the β-subunit of human Interleukin 12 (SEQ ID NO: 4), and
(b) introducing the obtained nucleic acid molecule of step (a) for expression into a suitable host cell or into a suitable cell extract or cell lysate.

In one embodiment, the present invention is directed to a method for producing the α-subunit of human Interleukin 35 (SEQ ID NO: 1) from human Interleukin 12, comprising the steps of:
(a) introducing into a nucleic acid molecule encoding the recombinant α- and β-subunits of the human Interleukin 12 polypeptide (SEQ ID NOs: 3 and 4) a nucleotide sequence mutating one amino acid residue of the β-subunit of human Interleukin 12 (SEQ ID NO: 4), and
(b) introducing the obtained nucleic acid molecule of step (a) for expression into a suitable host cell or into a suitable cell extract or cell lysate.

In one embodiment, the present invention is directed to a method for producing the β-subunit EBI3 of human Interleukin 35 (SEQ ID NO: 2) from human Interleukin 27, comprising the steps of:
(a) introducing into a nucleic acid molecule encoding the recombinant α- and β-subunits of the human Interleukin 27 polypeptide (SEQ ID NOs: 5 and 6) a nucleotide sequence mutating at least one amino acid residue of the α-subunit of human Interleukin 27 (SEQ ID NO: 5), and
(b) introducing the obtained nucleic acid molecule of step (a) for expression into a suitable host cell or into a suitable cell extract or cell lysate.

In one embodiment, the present invention is directed to a method for producing the β-subunit EBI3 of human Interleukin 35 (SEQ ID NO: 2) from human Interleukin 27, comprising the steps of:
(a) introducing into a nucleic acid molecule encoding the recombinant α- and β-subunits of the human Interleukin 27 polypeptide (SEQ ID NOs: 5 and 6) a nucleotide sequence mutating one amino acid residue of the α-subunit of human Interleukin 27 (SEQ ID NO: 5), and
(b) introducing the obtained nucleic acid molecule of step (a) for expression into a suitable host cell or into a suitable cell extract or cell lysate.

The protein sequence of the subunits of wild-type human Interleukin 35 as used herein refers to SEQ ID NOs: 1 and 2. IL-35 is composed of IL-12α and EBI3.

The polypeptide sequences of the subunits of wild-type human Interleukin 12, as used herein, refers to SEQ ID NOs: 3 and 4. In the context of the present invention, the term “α-subunit of human IL-12” or “α-subunit of human Interleukin 12” as used herein refers to the polypeptide sequence of SEQ ID NO: 3, which is also deposited under UniProtKB accession number P29459. In the context of the present invention, the term “β-subunit of human IL-12” or “β-subunit of human Interleukin 12”, as used herein, refers to the polypeptide sequence of SEQ ID NO: 4, which is also deposited under UniProtKB accession number P29460.

The protein sequence of the subunits of wild-type human Interleukin 27, as used herein, refers to SEQ ID NOs: 5 and 6. In the context of the present invention, it is noted that the term “α-subunit of human IL-27” or “α-subunit of human Interleukin 27” as used herein refers to the polypeptide sequence of SEQ ID NO: 5, which is also deposited under UniProtKB accession number Q8NEV9. The term “β-subunit of human IL-27” or “β-subunit of human Interleukin 27” or “EBI3”, as used herein, refers to the polypeptide sequence of SEQ ID NO: 6, which is also deposited under UniProtKB accession number Q14213. This subunit associates with the human IL-27 α-subunit to form the IL-27 Interleukin.

In a preferred embodiment of the method of the present invention, the method further comprises a step (d) comprising the isolation of human Interleukin 12 and/or the isolation of human Interleukin 27 from the product of step (c). The isolation of human Interleukin 12 and/or the isolation of human Interleukin 27 may be carried out by affinity chromatography.

It is also preferred for the method of the present invention that the method further comprises a step (e) comprising the separation of the α-subunit of human Interleukin 12 (SEQ ID NO: 3) from the isolated human Interleukin 12 of step (d) and/or the separation of the β-subunit of human Interleukin 27 (SEQ ID NO: 6) from the isolated human Interleukin 27 of step (d). The separation of the α-subunit of human Interleukin 12 (SEQ ID NO: 3) from the isolated human Interleukin 12 of step (d) and/or the separation of the β-subunit of human Interleukin 27 (SEQ ID NO: 6) from the isolated human Interleukin 27 of step (d) may be carried out by denaturation and subsequent renaturation under physiological conditions. It is preferred for step (e) of the present invention to use denaturing conditions to separate the α-subunit of human Interleukin 12 (SEQ ID NO: 3) from the isolated human Interleukin 12 of step (d) and/or to separate the β-subunit of human Interleukin 27 (EBI3, SEQ ID NO: 6) from the isolated human Interleukin 27. This means that it is preferred for step (e) of the present invention that the α-subunit of human Interleukin 12 (SEQ ID NO: 3) and/or the β-subunit of human Interleukin 27 (SEQ ID NO: 6) are denaturated and thereby separated from the β-subunit of human Interleukin 12 (SEQ ID NO: 4) and/or respectively from the β-subunit of human Interleukin 27 (SEQ ID NO: 6). It is further preferred for that embodiment that those subunits, the α-subunit of human Interleukin 12 (SEQ ID NO: 3) and/or the β-subunit of human Interleukin 27 (SEQ ID NO: 6), are subsequently refolded, more preferably refolded on a column, which may be used in step (f) of the method of the present invention, or refolded in solution.

In a preferred embodiment of the method of the present invention, the method further comprises a step (f) comprising the purification of the α-subunit of human Interleukin 12 (SEQ ID NO: 3) of step (e) and/or the purification of the β-subunit of human Interleukin 27 (SEQ ID NO: 6) of step (e). The purification of the α-subunit of human Interleukin 12 (SEQ ID NO: 3) of step (e) and/or the purification of the β-subunit of human Interleukin 27 (SEQ ID NO: 6) of step (e) may be carried out by size exclusion chromatography. However, any other chromatography method known to the person skilled in the art, like e.g. also immobilized metal affinity chromatography, may be applied in step (f) of the method of the present invention. Further, step (f) of the present invention preferably also comprises (an) enzymatic digestion step(s). The purification of the α-subunit of human Interleukin 12 (SEQ ID NO: 3) of step (e) and/or the purification of the β-subunit of human Interleukin 27 (SEQ ID NO: 6) of step (e) may comprise that one of said subunit(s) comprise(s) a tag.

It is preferred for the method of the present invention that step (a) comprises introducing into a nucleic acid molecule encoding the recombinant α- and β-subunits of the human Interleukin 12 polypeptide (SEQ ID NOs: 3 and 4) a nucleotide sequence mutating the amino acid residue of the α-subunit of human Interleukin 12 (SEQ ID NO: 3) at sequence position 96. It is also preferred for the method of the present invention that step (a) comprises introducing into a nucleic acid molecule encoding the recombinant α- and β-subunits of the human Interleukin 12 polypeptide (SEQ ID NOs: 3 and 4) a nucleotide sequence mutating the amino acid residue of the α-subunit of human Interleukin 12 (SEQ ID NO: 3) at sequence position 96 to any other amino acid than cysteine. It is further preferred for this embodiment that step (a) comprises introducing into a nucleic acid molecule encoding the recombinant α- and β-subunits of the human Interleukin 12 polypeptide (SEQ ID NOs: 3 and 4) a nucleotide sequence mutating the amino acid residue of the α-subunit of human Interleukin 12 (SEQ ID NO: 3) at sequence position 96 to serine. In the context of the present invention, it is noted that the term “C96S-mutation of the α-subunit of human IL-12” or “IL-12α^{C96S}” as used herein refers to the polypeptide sequence of SEQ ID NO: 7. The nucleotide sequence may further comprise a nucleotide sequence encoding an affinity tag.

In another preferred embodiment of the method of the present invention, step (a) comprises introducing into a nucleic acid molecule encoding the recombinant α- and β-subunits of the human Interleukin 12 polypeptide (SEQ ID NOs: 3 and 4) a nucleotide sequence mutating the amino acid residue of the β-subunit of human Interleukin 12 (SEQ ID NO: 4) at sequence position 199. It is also preferred for the method of the present invention that step (a) comprises introducing into a nucleic acid molecule encoding the recombinant α- and β-subunits of the human Interleukin 12 polypeptide (SEQ ID NOs: 3 and 4) a nucleotide sequence mutating the amino acid residue of the β-subunit of human Interleukin 12 (SEQ ID NO: 4) at sequence position 199 to any other amino acid than cysteine. It is further preferred for this embodiment that step (a) comprises introducing into a nucleic acid molecule encoding the recombinant α- and β-subunits of the human Interleukin 12 polypeptide (SEQ ID NOs: 3 and 4) a nucleotide sequence mutating the amino acid residue of the β-subunit of human Interleukin 12 (SEQ ID NO: 4) at sequence position 199 to serine. In the context of the present invention, it is noted that the term “C199S-mutation of the β-subunit of human IL-12” or “IL-12β^{C199S}” as used herein refers to the polypeptide sequence of SEQ ID NO: 8. The nucleotide sequence may further comprise a nucleotide sequence encoding an affinity tag.

In one embodiment of the method of the present invention, step (a) comprises introducing into a nucleic acid molecule encoding the recombinant α- and β-subunits of the human Interleukin 12 polypeptide (SEQ ID NOs: 3 and 4) a nucleotide sequence mutating the amino acid residue of the α-subunit of human Interleukin 12 (SEQ ID NO: 3) at sequence position 96 and introducing into a nucleic acid molecule encoding the recombinant α- and β-subunits of the human Interleukin 12 polypeptide (SEQ ID NOs: 3 and 4) a nucleotide sequence mutating the amino acid residue of the β-subunit of human Interleukin 12 (SEQ ID NO: 4) at sequence position 199. It is preferred for this embodiment that step (a) comprises introducing into a nucleic acid molecule encoding the recombinant α- and β-subunits of the human Interleukin 12 polypeptide (SEQ ID NOs: 3 and 4) a nucleotide sequence mutating the amino acid residue of the α-subunit of human Interleukin 12 (SEQ ID NO: 3) at sequence position 96 to serine and introducing into a nucleic acid molecule encoding the recombinant α- and β-subunits of the human Interleukin 12 polypeptide (SEQ ID NOs: 3 and 4) a nucleotide sequence mutating the amino acid residue of the β-subunit of human Interleukin 12 (SEQ ID NO: 4) at sequence position 199 to serine.

In one embodiment of the method of the present invention, step (b) comprises introducing into a nucleic acid molecule encoding the recombinant α- and β-subunits of the human Interleukin 27 polypeptide (SEQ ID NOs: 5 and 6) a nucleotide sequence mutating the amino acid residue of the α-subunit of human Interleukin 27 (SEQ ID NO: 5) at sequence position 162. It is preferred for this embodiment that step (b) comprises introducing into a nucleic acid molecule encoding the recombinant α- and β-subunits of the human Interleukin 27 polypeptide (SEQ ID NOs: 5 and 6) a nucleotide sequence mutating the amino acid residue of the α-subunit of human Interleukin 27 (SEQ ID NO: 5) at sequence position 162 to cysteine. In the context of the present invention, it is noted that the term “L162C-mutation of the α-subunit of human IL-27” or “IL-27α^{L162C}” as used herein refers to the polypeptide sequence of SEQ ID NO: 9. The nucleotide sequence may further comprise a nucleotide sequence encoding an affinity tag.

It is also within the scope of the present invention that the method for producing the α-subunit of human Interleukin 35 (SEQ ID NO: 1) from human Interleukin 12 and/or the β-subunit EBI3 of human Interleukin 35 (SEQ ID NO: 2) from human Interleukin 27, comprises the steps of:
(a) introducing into a nucleic acid molecule encoding the recombinant α- and β-subunits of the human Interleukin 12 polypeptide (SEQ ID NOs: 3 and 4) a nucleotide sequence mutating at least one, preferably one, amino acid residue of the α-subunit of human Interleukin 12 (SEQ ID NO: 3) and/or mutating at least one, preferably one, amino acid residue of the β-subunit of human Interleukin 12 (SEQ ID NO: 4), and/or
(b) introducing into a nucleic acid molecule encoding the recombinant α- and β-subunits of the human Interleukin 27 polypeptide (SEQ ID NOs: 5 and 6) the nucleotide sequence encoding the wild type of the α-subunit of human Interleukin 27 (SEQ ID NO: 5), and
(c) introducing the obtained nucleic acid molecule(s) of steps (a) and/or (b) for expression into a suitable host cell or into a suitable cell extract or cell lysate.

It is also within the scope of the present invention that the method for producing the α-subunit of human Interleukin 35 (SEQ ID NO: 1) from human Interleukin 12 and/or the β-subunit EBI3 of human Interleukin 35 (SEQ ID NO: 2) from human Interleukin 27, comprises the steps of:
(a) introducing into a nucleic acid molecule encoding the recombinant α- and β-subunits of the human Interleukin 12 polypeptide (SEQ ID NOs: 3 and 4) a nucleotide sequence encoding the wild type of the α-subunit of human Interleukin 12 (SEQ ID NO: 3) and a nucleotide sequence mutating at least one, preferably one, amino acid residue of the β-subunit of human Interleukin 12 (SEQ ID NO: 4), and/or
(b) introducing into a nucleic acid molecule encoding the recombinant α- and β-subunits of the human Interleukin 27 polypeptide (SEQ ID NOs: 5 and 6) a nucleotide sequence mutating at least one, preferably one, amino acid residue of the α-subunit of human Interleukin 27 (SEQ ID NO: 5), and
(c) introducing the obtained nucleic acid molecule(s) of steps (a) and/or (b) for expression into a suitable host cell or into a suitable cell extract or cell lysate.

It is also within the scope of the present invention that the method for producing the α-subunit of human Interleukin 35 (SEQ ID NO: 1) from human Interleukin 12 and/or the β-subunit EBI3 of human Interleukin 35 (SEQ ID NO: 2) from human Interleukin 27, comprises the steps of:
(a) introducing into a nucleic acid molecule encoding the recombinant α- and β-subunits of the human Interleukin 12 polypeptide (SEQ ID NOs: 3 and 4) a nucleotide sequence mutating at least one, preferably one, amino acid residue of the α-subunit of human Interleukin 12 (SEQ ID NO: 3) and a nucleotide sequence encoding the wild type of the β-subunit of human Interleukin 12 (SEQ ID NO: 4), and/or
(b) introducing into a nucleic acid molecule encoding the recombinant α- and β-subunits of the human Interleukin 27 polypeptide (SEQ ID NOs: 5 and 6) a nucleotide sequence mutating at least one, preferably one, amino acid residue of the α-subunit of human Interleukin 27 (SEQ ID NO: 5), and
(c) introducing the obtained nucleic acid molecule(s) of steps (a) and/or (b) for expression into a suitable host cell or into a suitable cell extract or cell lysate.

It is also within the scope of the present invention that the method for producing the α-subunit of human Interleukin 35 (SEQ ID NO: 1) from human Interleukin 12 comprises the steps of:
(a) introducing into a nucleic acid molecule encoding the recombinant α- and β-subunits of the human Interleukin 12 polypeptide (SEQ ID NOs: 3 and 4) a nucleotide sequence mutating at least one, preferably one, amino acid residue of the α-subunit of human Interleukin 12 (SEQ ID NO: 3) and mutating at least one, preferably one, amino acid residue of the β-subunit of human Interleukin 12 (SEQ ID NO: 4), and
(b) introducing the obtained nucleic acid molecule of step (a) for expression into a suitable host cell or into a suitable cell extract or cell lysate.

It is also within the scope of the present invention that the method for producing the α-subunit of human Interleukin 35 (SEQ ID NO: 1) from human Interleukin 12 comprises the steps of:
(a) introducing into a nucleic acid molecule encoding the recombinant α- and β-subunits of the human Interleukin 12 polypeptide (SEQ ID NOs: 3 and 4) a nucleotide sequence encoding the wild type of the α-subunit of human Interleukin 12 (SEQ ID NO: 3) and a nucleotide sequence mutating at least one, preferably one, amino acid residue of the β-subunit of human Interleukin 12 (SEQ ID NO: 4), and
(c) introducing the obtained nucleic acid molecule(s) of steps (a) and/or (b) for expression into a suitable host cell or into a suitable cell extract or cell lysate.

It is also within the scope of the present invention that the method for producing the α-subunit of human Interleukin 35 (SEQ ID NO: 1) from human Interleukin 12 comprises the steps of:
(a) introducing into a nucleic acid molecule encoding the recombinant α- and β-subunits of the human Interleukin 12 polypeptide (SEQ ID NOs: 3 and 4) a nucleotide sequence mutating at least one, preferably one, amino acid residue of the α-subunit of human Interleukin 12 (SEQ ID NO: 3) and a nucleotide sequence encoding the wild type of the β-subunit of human Interleukin 12 (SEQ ID NO: 4), and
(b) introducing the obtained nucleic acid molecule of step (a) for expression into a suitable host cell or into a suitable cell extract or cell lysate.

It is also within the scope of the present invention that the method for producing the β-subunit EBI3 of human Interleukin 35 (SEQ ID NO: 2) from human Interleukin 27, comprises the steps of:
(a) introducing into a nucleic acid molecule encoding the recombinant α- and β-subunits of the human Interleukin 27 polypeptide (SEQ ID NOs: 5 and 6) a nucleotide sequence mutating at least one, preferably one, amino acid residue of the α-subunit of human Interleukin 27 (SEQ ID NO: 5), and
(b) introducing the obtained nucleic acid molecule of step (a) for expression into a suitable host cell or into a suitable cell extract or cell lysate.

In one embodiment of the present invention, steps (a) and (b) according to the method of the present invention are carried out simultaneously or step (a) is carried out before step (b) or step (b) is carried out before step (a). In one embodiment of the present invention, only step (a) and not step (b) according to the method of the present invention is carried out. In one embodiment of the present invention, only step (b) and not step (a) according to the method of the present invention is carried out.

In yet another embodiment of the present invention, step (a) comprises introducing into a nucleic acid molecule encoding the recombinant α- and β-subunits of the human Interleukin 12 polypeptide (SEQ ID NOs: 3 and 4) a nucleotide sequence mutating the amino acid residue of the α-subunit of human Interleukin 12 (SEQ ID NO: 3) at sequence position 96, and step (a) comprises introducing into a nucleic acid molecule encoding the recombinant α- and β-subunits of the human Interleukin 12 polypeptide (SEQ ID NOs: 3 and 4) a nucleotide sequence mutating the amino acid residue of the β-subunit of human Interleukin 12 (SEQ ID NO: 4) at sequence position 199, and step (b) comprises introducing into a nucleic acid molecule encoding the recombinant α- and β-subunits of the human Interleukin 27 polypeptide (SEQ ID NOs: 5 and 6) a nucleotide sequence mutating the amino acid residue of the α-subunit of human Interleukin 27 (SEQ ID NO: 5) at sequence position 162. It is preferred for this embodiment of the present invention that step (a) comprises introducing into a nucleic acid molecule encoding the recombinant α- and β-subunits of the human Interleukin 12 polypeptide (SEQ ID NOs: 3 and 4) a nucleotide sequence mutating the amino acid residue of the α-subunit of human Interleukin 12 (SEQ ID NO: 3) at sequence position 96 to serine, and step (a) comprises introducing into a nucleic acid molecule encoding the recombinant α- and β-subunits of the human Interleukin 12 polypeptide (SEQ ID NOs: 3 and 4) a nucleotide sequence mutating the amino acid residue of the β-subunit of human Interleukin 12 (SEQ ID NO: 4) at sequence position 199 to serine, and step (b) comprises introducing into a nucleic acid molecule encoding the recombinant α- and β-subunits of the human Interleukin 27 polypeptide (SEQ ID NOs: 5 and 6) a nucleotide sequence mutating the amino acid residue of the α-subunit of human Interleukin 27 (SEQ ID NO: 5) at sequence position 162 to cysteine.

The present invention additionally provides an α-subunit of human Interleukin 12 (SEQ ID NO: 3) obtained according to the method of the present invention. In one embodiment, said α-subunit of human Interleukin 12 (SEQ ID NO: 3) is identical to the α-subunit of human Interleukin 35 (SEQ ID NO: 1). It is preferred for this aspect of the present invention that in said α-subunit of human Interleukin 12 (SEQ ID NO: 3) the amino acid residue at amino acid position 96 is mutated to serine (SEQ ID NO: 7).

In a further aspect, the present invention also provides a β-subunit of human Interleukin 27 (SEQ ID NO: 6) obtained according to the method of the present invention. In one embodiment, said β-subunit of human Interleukin 27 (SEQ ID NO: 6) is identical to the β-subunit of human Interleukin 35 (SEQ ID NO: 2).

In a further aspect, the present invention provides a nucleic acid molecule comprising a nucleotide sequence encoding the α-subunit of human Interleukin 12 (SEQ ID NO: 3) obtained according to the method of the present invention.

The present invention additionally provides a nucleic acid molecule comprising a nucleotide sequence encoding the β-subunit of human Interleukin 27 (SEQ ID NO: 6) obtained according to the method of the present invention.

It is preferred, that the nucleic acid molecule of the present invention is operably linked to a regulatory sequence to allow expression of the nucleic acid molecule. This regulatory sequence may comprise a promoter sequence. The term “promoter” or “promoter sequence” means a DNA sequence, which initiates and directs the transcription of a gene into an RNA transcript in cells.

The nucleic acid molecule(s) according to the present invention may be comprised in a vector.

As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome.

In yet another aspect, the invention provides the nucleic acid molecule(s) according to the present invention and as described herein for use as a therapeutic agent.

The present invention also provides a host cell containing a nucleic acid molecule of the present invention as described above. A host cell can be any prokaryotic (e.g., E. coli) or eukaryotic cell (e.g., insect cells, yeast or mammalian cells).

The present invention also provides an immune modulator comprising a subunit obtained according to the method of the present invention. An immune modulator is any protein, substance or composition that is able to carry out immunomodulation, which is the adjustment of the immune-response to a desired level, as e.g. in immunopotentiation, immunosuppression, or induction of immunologic tolerance.

The present invention also provides the use of the α-subunit of human Interleukin 35 (SEQ ID NO: 1) and/or of the β-subunit of human Interleukin 35 (SEQ ID NO: 2), preferably of a subunit obtained according to the method of the present invention, for the manufacture of a medicament for treating a disease in a mammal, preferably a human. Suitable diseases include, but are not limited to, an infectious disease, an autoimmune disease, cancer, preferably breast cancer, pancreatic cancer or lung cancer, a transplantation-related disease, preferably Graft-versus-Host-disease, an inflammatory disease, preferably a chronic inflammatory disease or an acute inflammatory disease, more preferably a chronic inflammatory bowel disease, sepsis, septic shock, diabetes or an allergic disease, preferably asthma in a mammal.

The present invention also provides the α-subunit of human Interleukin 35 (SEQ ID NO: 1) and/or the β-subunit of human Interleukin 35 (SEQ ID NO: 2), preferably of a subunit obtained according to the method of the present invention, for use as a medicament. The present invention also provides the α-subunit of human Interleukin 35 (SEQ ID NO: 1) and/or the β-subunit of human Interleukin 35 (SEQ ID NO: 2), preferably of a subunit obtained according to the method of the present invention, for use as drug. Thus, any of the said subunits, preferably those obtained according to the method of the present invention, may be used as a drug. It is also preferred for this aspect of the present invention that any of these subunits are for use in the treatment of a disease in a mammal. Such a disease may be, but is not limited to, an infectious disease, an autoimmune disease, cancer, preferably breast cancer, pancreatic cancer or lung cancer, a transplantation-related disease, preferably Graft-versus-Host-disease, an inflammatory disease, preferably a chronic inflammatory disease or an acute inflammatory disease, more preferably a chronic inflammatory bowel disease, sepsis, septic shock, diabetes or an allergic disease, preferably asthma.

The present invention also provides a method of treating an Interleukin 35-mediated disease, comprising the step of administering a composition comprising the α-subunit and/or the β-subunit of human Interleukin 35 (SEQ ID NOs: 1 and 2), preferably any of those subunits obtained according to the method of the present invention. The present invention also provides a method of treating an Interleukin 35-mediated disease, comprising the step of administering the α-subunit and/or the β-subunit of human Interleukin 35 (SEQ ID NOs: 1 and 2), preferably any of those subunits obtained according to the method of the present invention. The present invention also provides a method of treating an Interleukin 35-mediated disease, comprising the step of administering the α-subunit of human Interleukin 35 (SEQ ID NO: 1), preferably that α-subunit of human Interleukin 35 (SEQ ID NO: 1) obtained according to the method of the present invention. The present invention also provides a method of treating an Interleukin 35-mediated disease, comprising the step of administering the β-subunit of human Interleukin 35 (SEQ ID NO: 2), preferably that β-subunit of human Interleukin 35 (SEQ ID NO: 1) obtained according to the method of the present invention. The Interleukin 35-mediated disease may be, but is not limited to, an infectious disease, an autoimmune disease, cancer, preferably breast cancer, pancreatic cancer or lung cancer, a transplantation-related disease, preferably Graft-versus-Host-disease, an inflammatory disease, preferably a chronic inflammatory disease or an acute inflammatory disease, more preferably a chronic inflammatory bowel disease, sepsis, septic shock, diabetes or an allergic disease, preferably asthma. The term “Interleukin 35-mediated disease” can be used interchangeably herein with the term “Interleukin 35-related disease” and means any disease wherein IL-35 regulates the development and/or progression of that disease.

The present invention also provides the use of the α-subunit of human Interleukin 35 (SEQ ID NO: 1) and/or of the β-subunit of human Interleukin 35 (SEQ ID NO: 2), preferably of any of those subunits obtained according to the method of the present invention, for producing (a) binding reagent(s) against any of said subunit(s). The term “against” means herein that the binding reagent(s) specifically bind to the α-subunit of human Interleukin 35 (SEQ ID NO: 1) and/or to the β-subunit of human Interleukin 35 (SEQ ID NO: 2).

A further aspect of the present invention is directed to a method for producing (a) binding reagent(s) against the α-subunit of human Interleukin 35 (SEQ ID NO: 1) and/or against the β-subunit of human Interleukin 35 (SEQ ID NO: 2), preferably against any subunit obtained according to the method of the present invention. It is preferred for this method of the present invention that the method comprises immunizing an animal with any of said subunits(s). In one embodiment of this method for producing (a) binding reagent(s), said method comprises the in vitro generation of the binding reagent(s) against any of said subunit(s) according to the present invention. It is also preferred for this method for producing (a) binding reagent(s) of the present invention that the method comprises the selection of the binding reagent(s) specific for any of said subunit(s) according to the present invention, preferably comprising the selection of the binding reagent(s) specific for any of said subunit(s) according to the present invention from a pool of binding reagents. It is preferred for said method of producing (a) binding reagent(s) against the α-subunit of human Interleukin 35 (SEQ ID NO: 1) and/or against the β-subunit of human Interleukin 35 (SEQ ID NO: 2) that said binding reagent(s) may be selected from the group consisting of an antibody, a nanobody, a divalent antibody fragment, a monovalent antibody fragment, a proteinaceous binding molecule with antibody-like binding properties and an MHC molecule, preferably wherein the divalent antibody fragment is an (Fab)2′-fragment, or a divalent single-chain Fv fragment, and/or preferably wherein the monovalent antibody fragment is selected from the group consisting of a Fab fragment, a Fv fragment, and a single-chain Fv fragment (scFv) and/or preferably wherein the proteinaceous binding molecule with antibody-like binding properties is selected from the group consisting of an aptamer, a mutein based on a polypeptide of the lipocalin family, a glubody, a protein based on the ankyrin scaffold, a protein based on the crystalline scaffold, an adnectin, an avimer, a EGF-like domain, a Kringle-domain, a fibronectin type I domain, a fibronectin type II domain, a fibronectin type III domain, a PAN domain, a G1a domain, a SRCR domain, a Kunitz/Bovine pancreatic trypsin Inhibitor domain, tendamistat, a Kazal-type serine protease inhibitor domain, a Trefoil (P-type) domain, a von Willebrand factor type C domain, an Anaphylatoxin-like domain, a CUB domain, a thyroglobulin type I repeat, LDL-receptor class A domain, a Sushi domain, a Link domain, a Thrombospondin type I domain, an immunoglobulin domain or an immunoglobulin-like domain, a C-type lectin domain, a MAM domain, a von Willebrand factor type A domain, a Somatomedin B domain, a WAP-type four disulfide core domain, a F5/8 type C domain, a Hemopexin domain, an SH2 domain, an SH3 domain, a Laminin-type EGF-like domain, a C2 domain, a nanobody, a adnectin, a tetranectin, a microbody, an affilin, an affibody or an ankyrin, a crystallin, a knottin, ubiquitin, a zinc-finger protein, an autofluorescent protein, an ankyrin or ankyrin repeat protein or a leucine-rich repeat protein, and an avimer.

Antibodies, including monoclonal antibodies (mAb), can be made by standard protocols (see, for example, Harlow and Lane, Using Antibodies: A Laboratory Manual, CSHL, New York, 1999). Briefly, a mammal such as a mouse, hamster or rabbit can be immunized with an immunogenic form of a peptide. Techniques for conferring immunogenicity on a protein or peptide may include conjugation to carriers or other techniques, well known in the art.

The antibodies produced according to the method of the present invention include antibodies that specifically bind IL-35 or any of the subunits of IL-35. By “antibodies that specifically bind” is intended that the antibodies will not substantially cross react with another polypeptide. By “not substantially cross react” is intended that the antibody or fragment has a binding affinity for a non-homologous protein, which is less than 10%, more preferably less than 5%, and even more preferably less than 1%, of the binding affinity for IL-35 or any of the subunits of IL-35.

The method for producing (a) binding reagent(s) according to the present invention may further comprise the use of polyclonal sera, which may be prepared by conventional methods. In general, when the binding reagent(s) is/are an antibody/antibodies, a solution containing the respective IL-35- or IL-35-subunit(s) antigen(s) is first used to immunize a suitable animal, preferably a mouse, rat, rabbit, or goat. Rabbits or goats are preferred for the preparation of polyclonal sera due to the volume of serum obtainable, and the availability of labeled anti-rabbit and anti-goat antibodies. Polyclonal sera can be prepared in a transgenic animal, preferably a mouse bearing human immunoglobulin loci. Immunization can also be performed by mixing or emulsifying the antigen-containing solution in saline, preferably in an adjuvant such as Freud's complete adjuvant, and injecting the mixture or emulsion parenterally. A dose of 50-200 μg/injection is typically sufficient. Immunization is generally boosted 2-6 weeks later with one or more injections of the protein in saline, preferably using Freund's incomplete adjuvant. One may alternatively generate antibodies by in vitro immunization using methods known in the art, which may be considered equivalent to in vitro immunization. Polyclonal antisera may be obtained by bleeding the immunized animal into a glass or plastic container, incubating the blood at 25° C. for one hour, followed by incubating at 4° C. for 2-18 hours. The serum is recovered by centrifugation (e.g. 1000×g for 10 minutes). About 20-50 ml per bleed may be obtained from rabbits.

In a further aspect, the present invention is directed to a binding reagent which specifically binds to the α-subunit of human Interleukin 35 (SEQ ID NO: 1) and/or the β-subunit of human Interleukin 35 (SEQ ID NO: 2). The binding reagent may be selected from the group consisting of an antibody, a nanobody, a divalent antibody fragment, a monovalent antibody fragment, a proteinaceous binding molecule with antibody-like binding properties and an MHC molecule, preferably wherein the divalent antibody fragment is an (Fab)2′-fragment, or a divalent single-chain Fv fragment, and/or preferably the monovalent antibody fragment is selected from the group consisting of a Fab fragment, a Fv fragment, and a single-chain Fv fragment (scFv) and/or preferably wherein the proteinaceous binding molecule with antibody-like binding properties is selected from the group of an aptamer, a mutein based on a polypeptide of the lipocalin family, a glubody, a protein based on the ankyrin scaffold, a protein based on the crystalline scaffold, an adnectin, an avimer, a EGF-like domain, a Kringle-domain, a fibronectin type I domain, a fibronectin type II domain, a fibronectin type III domain, a PAN domain, a G1a domain, a SRCR domain, a Kunitz/Bovine pancreatic trypsin Inhibitor domain, tendamistat, a Kazal-type serine protease inhibitor domain, a Trefoil (P-type) domain, a von Willebrand factor type C domain, an Anaphylatoxin-like domain, a CUB domain, a thyroglobulin type I repeat, LDL-receptor class A domain, a Sushi domain, a Link domain, a Thrombospondin type I domain, an immunoglobulin domain or an immunoglobulin-like domain, a C-type lectin domain, a MAM domain, a von Willebrand factor type A domain, a Somatomedin B domain, a WAP-type four disulfide core domain, a F5/8 type C domain, a Hemopexin domain, an SH2 domain, an SH3 domain, a Laminin-type EGF-like domain, a C2 domain, a nanobody, a adnectin, a tetranectin, a microbody, an affilin, an affibody or an ankyrin, a crystallin, a knottin, ubiquitin, a zinc-finger protein, an autofluorescent protein, an ankyrin or ankyrin repeat protein or a leucine-rich repeat protein, and an avimer.

Sequences, as used herein, are depicted in below Table 1

| SEQ ID NO: | Name | Sequence |
|---|---|---|
| 1 | human IL-35α | MCPARSLLLVATLVLLDHLSLARNLPVA |
| | subunit | TPDPGMFPCLHHSQNLLRAVSNMLQK |
| | | ARQTLEFYPCTSEEIDHEDITKDKTSTV |
| | | EACLPLELTKNESCLNSRETSFITNGSC |
| | | LASRKTSFMMALCLSSIYEDLKMYQVE |
| | | FKTMNAKLLMDPKRQIFLDQNMLAVID |
| | | ELMQALNFNSETVPQKSSLEEPDFYKT |
| | | KIKLCILLHAFRIRAVTIDRVMSYLNAS |
| 2 | human IL-35β | MTPQLLLALVLWASCPPCSGRKGPPA |
| | subunit | ALTLPRVQCRASRYPIAVDCSWTLPPA |
| | | PNSTSPVSFIATYRLGMAARGHSWPCL |
| | | QQTPTSTSCTITDVQLFSMAPYVLNVT |
| | | AVHPWGSSSSFVPFITEHIIKPDPPEGV |
| | | RLSPLAERQLQVQWEPPGSWPFPEIFS |
| | | LKYWIRYKRQGAARFHRVGPIEATSFIL |
| | | RAVRPRARYYVQVAAQDLTDYGELSD |
| | | WSLPATATMSLGK |
| 3 | IL-12α | MCPARSLLLVATLVLLDHLSLARNLPVA |
| | | TPDPGMFPCLHHSQNLLRAVSNMLQK |
| | | ARQTLEFYPCTSEEIDHEDITKDKTSTV |
| | | EACLPLELTKNESCLNSRETSFITNGSC |
| | | LASRKTSFMMALCLSSIYEDLKMYQVE |
| | | FKTMNAKLLMDPKRQIFLDQNMLAVID |
| | | ELMQALNFNSETVPQKSSLEEPDFYKT |
| | | KIKLCILLHAFRIRAVTIDRVMSYLNAS |
| 4 | IL-12β | MCHQQLVISWFSLVFLASPLVAIWELKK |
| | | DVYVVELDWYPDAPGEMVVLTCDTPE |
| | | EDGITWTLDQSSEVLGSGKTLTIQVKEF |
| | | GDAGQYTCHKGGEVLSHSLLLLHKKED |
| | | GIWSTDILKDQKEPKNKTFLRCEAKNY |
| | | SGRFTCWWLTTISTDLTFSVKSSRGSS |
| | | DPQGVTCGAATLSAERVRGDNKEYEY |
| | | SVECQEDSACPAAEESLPIEVMVDAVH |
| | | KLKYENYTSSFFIRDIIKPDPPKNLQLKP |
| | | LKNSRQVEVSWEYPDTWSTPHSYFSL |
| | | TFCVQVQGKSKREKKDRVFTDKTSATV |
| | | ICRKNASISVRAQDRYYSSSWSEWASV |
| | | PCS |
| 5 | IL-27α | MGQTAGDLGWRLSLLLLPLLLVQAGV |
| | | WGFPRPPGRPQLSLQELRREFTVSLHL |
| | | ARKLLSEVRGQAHRFAESHLPGVNLYL |
| | | LPLGEQLPDVSLTFQAWRRLSDPERLC |
| | | FISTTLQPFHALLGGLGTQGRWTNMER |
| | | MQLWAMRLDLRDLQRHLRFQVLAAGF |
| | | NLPEEEEEEEEEEEEERKGLLPGALGS |
| | | ALQGPAQVSWPQLLSTYRLLHSLELVL |
| | | SRAVRELLLLSKAGHSVWPLGFPTLSP |
| | | QP |
| 6 | IL-27β = EBI3 | MTPQLLLALVLWASCPPCSGRKGPPA |
| | | ALTLPRVQCRASRYPIAVDCSWTLPPA |
| | | PNSTSPVSFIATYRLGMAARGHSWPCL |
| | | QQTPTSTSCTITDVQLFSMAPYVLNVT |
| | | AVHPWGSSSSFVPFITEHIIKPDPPEGV |
| | | RLSPLAERQLQVQWEPPGSWPFPEIFS |
| | | LKYWIRYKRQGAARFHRVGPIEATSFIL |
| | | RAVRPRARYYVQVAAQDLTDYGELSD |
| | | WSLPATATMSLGK |
| 7 | IL-12α^{C96S} | MCPARSLLLVATLVLLDHLSLARNLPVA |
| | | TPDPGMFPCLHHSQNLLRAVSNMLQK |
| | | ARQTLEFYPCTSEEIDHEDITKDKTSTV |
| | | EACLPLELTKNESSLNSRETSFITNGSC |
| | | LASRKTSFMMALCLSSIYEDLKMYQVE |
| | | FKTMNAKLLMDPKRQIFLDQNMLAVID |
| | | ELMQALNFNSETVPQKSSLEEPDFYKT |
| | | KIKLCILLHAFRIRAVTIDRVMSYLNAS |
| 8 | IL-12β^{C199S} | MCHQQLVISWFSLVFLASPLVAIWELKK |
| | | DVYVVELDWYPDAPGEMVVLTCDTPE |
| | | EDGITWTLDQSSEVLGSGKTLTIQVKEF |
| | | GDAGQYTCHKGGEVLSHSLLLLHKKED |
| | | GIWSTDILKDQKEPKNKTFLRCEAKNY |
| | | SGRFTCWWLTTISTDLTFSVKSSRGSS |
| | | DPQGVTCGAATLSAERVRGDNKEYEY |
| | | SVECQEDSASPAAEESLPIEVMVDAVH |
| | | KLKYENYTSSFFIRDIIKPDPPKNLQLKP |
| | | LKNSRQVEVSWEYPDTWSTPHSYFSL |
| | | TFCVQVQGKSKREKKDRVFTDKTSATV |
| | | ICRKNASISVRAQDRYYSSSWSEWASV |
| | | PCS |
| 9 | IL-27α^{L162C} | MGQTAGDLGWRLSLLLLPLLLVQAGV |
| | | WGFPRPPGRPQLSLQELRREFTVSLHL |
| | | ARKLLSEVRGQAHRFAESHLPGVNLYL |
| | | LPLGEQLPDVSLTFQAWRRLSDPERLC |
| | | FISTTLQPFHALLGGLGTQGRWTNMER |
| | | MQLWAMRLDLRDLQRHLRFQVLAAGF |
| | | NCPEEEEEEEEEEEEERKGLLPGALGS |
| | | ALQGPAQVSWPQLLSTYRLLHSLELVL |
| | | SRAVRELLLLSKAGHSVWPLGFPTLSP |
| | | QP |
| 10 | 18S primer forward | GTAACCCGTTGAACCCCATT |
| | sequence | |
| 11 | 18S primer reverse | CCATCCATTCGGTAGTAGCG |
| | sequence | |
| 12 | ACTB primer | GGATGCAGAAGGAGATCACT |
| | forward sequence | |
| 13 | ACTB primer | CGATCCACACGGAGTACTTG |
| | reverse sequence | |
| 14 | CXCL8 primer | GAAGTTTTTGAAGAGGGCTGAGA |
| | forward sequence | |
| 15 | CXCL8 primer | TGCTTGAAGTTTCACTGGCAT |
| | reverse sequence | |
| 16 | GAPDH primer | GAAGGTGAAGGTCGGAGT |
| | forward sequence | |
| 17 | GAPDH primer | GAAGATGGTGATGGGATTTC |
| | reverse sequence | |
| 18 | IL1B primer forward | AGAAGTACCTGAGCTCGCCA |
| | sequence | |
| 19 | IL1B primer reverse | CTGGAAGGAGCACTTCATCTGT |
| | sequence | |
| 20 | IL6 primer forward | ACATGTGTGAAAGCAGCAAAG |
| | sequence | |
| 21 | IL6 primer reverse | GGCAAGTCTCCTCATTGAATCC |
| | sequence | |
| 22 | TNF primer forward | CCCATGTTGTAGCAAACCCTC |
| | sequence | |
| 23 | TNF primer reverse | TATCTCTCAGCTCCACGCCA |
| | sequence | |
| 24 | TGFB primer | CACGCAGTACAGCAAGGTCC |
| | forward sequence | |
| 25 | TGFB primer | CCACGTAGTACACGATGGGC |
| | reverse sequence | |
| 26 | IL5 primer forward | TCTCCAGTGTGCCTATTCCC |
| | sequence | |
| 27 | IL5 primer reverse | CGAACTCTGCTGATAGCCAA |
| | sequence | |
| 28 | IL-12α^{KDEL} | MCPARSLLLVATLVLLDHLSLARNLPVA |
| | | TPDPGMFPCLHHSQNLLRAVSNMLQK |
| | | ARQTLEFYPCTSEEIDHEDITKDKTSTV |
| | | EACLPLELTKNESCLNSRETSFITNGSC |
| | | LASRKTSFMMALCLSSIYEDLKMYQVE |
| | | FKTMNAKLLMDPKRQIFLDQNMLAVID |
| | | ELMQALNFNSETVPQKSSLEEPDFYKT |
| | | KIKLCILLHAFRIRAVTIDRVMSYLNASG |
| | | SGSGSKDEL |
| 29 | EBI3^{KDEL} | MTPQLLLALVLWASCPPCSGRKGPPA |
| | | ALTLPRVQCRASRYPIAVDCSWTLPPA |
| | | PNSTSPVSFIATYRLGMAARGHSWPCL |
| | | QQTPTSTSCTITDVQLFSMAPYVLNVT |
| | | AVHPWGSSSSFVPFITEHIIKPDPPEGV |
| | | RLSPLAERQLQVQWEPPGSWPFPEIFS |
| | | LKYWIRYKRQGAARFHRVGPIEATSFIL |
| | | RAVRPRARYYVQVAAQDLTDYGELSD |
| | | WSLPATATMSLGKGSGSGSKDEL |
| 30 | IL-12β^{C199S, KDEL} | MCHQQLVISWFSLVFLASPLVAIWELKK |
| | | DVYVVELDWYPDAPGEMVVLTCDTPE |
| | | EDGITWTLDQSSEVLGSGKTLTIQVKEF |
| | | GDAGQYTCHKGGEVLSHSLLLLHKKED |
| | | GIWSTDILKDQKEPKNKTFLRCEAKNY |
| | | SGRFTCWWLTTISTDLTFSVKSSRGSS |
| | | DPQGVTCGAATLSAERVRGDNKEYEY |
| | | SVECQEDSASPAAEESLPIEVMVDAVH |
| | | KLKYENYTSSFFIRDIIKPDPPKNLQLKP |
| | | LKNSRQVEVSWEYPDTWSTPHSYFSL |
| | | TFCVQVQGKSKREKKDRVFTDKTSATV |
| | | ICRKNASISVRAQDRYYSSSWSEWASV |
| | | PCSGSGSGSKDEL |
| 31 | IL-27α^{KDEL} | MGQTAGDLGWRLSLLLLPLLLVQAGV |
| | | WGFPRPPGRPQLSLQELRREFTVSLHL |
| | | ARKLLSEVRGQAHRFAESHLPGVNLYL |
| | | LPLGEQLPDVSLTFQAWRRLSDPERLC |
| | | FISTTLQPFHALLGGLGTQGRWTNMER |
| | | MQLWAMRLDLRDLQRHLRFQVLAAGF |
| | | NLPEEEEEEEEEEEEERKGLLPGALGS |
| | | ALQGPAQVSWPQLLSTYRLLHSLELVL |
| | | SRAVRELLLLSKAGHSVWPLGFPTLSP |
| | | QPGSGSGSKDEL |
| 32 | IL-12α^{C96S, His} | MCPARSLLLVATLVLLDHLSLARNLPVA |
| | | TPDPGMFPCLHHSQNLLRAVSNMLQK |
| | | ARQTLEFYPCTSEEIDHEDITKDKTSTV |
| | | EACLPLELTKNESSLNSRETSFITNGSC |
| | | LASRKTSFMMALCLSSIYEDLKMYQVEFK |
| | | TMNAKLLMDPKRQIFLDQNMLAVIDELMQ |
| | | ALNFNSETVPQKSSLEEPDFYKTKIKLCI |
| | | LLHAFRIRAVTIDRVMSYLNASGGENLYF |
| | | QSHHHHHH |
| 33 | mIL-27α^{His} | FPTDPLSLQELRREFTVSLYLARKLLSE |
| | | VQGYVHSFAESRLPGVNLDLLPLGYHL |
| | | PNVSLTFQAWHHLSDSERLCFLATTLR |
| | | PFPAMLGGLGTQGTWTSSEREQLWA |
| | | MRLDLRDLHRHLRFQVLAAGFKCSKEE |
| | | EDKEEEEEEEEEEKKLPLGALGGPNQV |
| | | SSQVSWPQLLYTYQLLHSLELVLSRAV |
| | | RDLLLLSLPRRPGSAWDSHHHHHHHH |
| | | HH |
| 34 | IL27α^{L162C, His} | MGQTAGDLGWRLSLLLLPLLLVQAGV |
| | | WGFPRPPGRPQLSLQELRREFTVSLHL |
| | | ARKLLSEVRGQAHRFAESHLPGVNLYL |
| | | LPLGEQLPDVSLTFQAWRRLSDPERLC |
| | | FISTTLQPFHALLGGLGTQGRWTNMER |
| | | MQLWAMRLDLRDLQRHLRFQVLAAGFN |
| | | CPEEEEEEEEEEEEERKGLLPGALGSA |
| | | LQGPAQVSWPQLLSTYRLLHSLELVLS |
| | | RAVRELLLLSKAGHSVWPLGFPTLSPQ |
| | | PGGLEVLFQGPGGGGHHHHHH |
| 35 | IL12β^{C199S, His} | MCHQQLVISWFSLVFLASPLVAIWELKK |
| | | DVYVVELDWYPDAPGEMVVLTCDTPE |
| | | EDGITWTLDQSSEVLGSGKTLTIQVKEF |
| | | GDAGQYTCHKGGEVLSHSLLLLHKKED |
| | | GIWSTDILKDQKEPKNKTFLRCEAKNY |
| | | SGRFTCWWLTTISTDLTFSVKSSRGSS |
| | | DPQGVTCGAATLSAERVRGDNKEYEY |
| | | SVECQEDSASPAAEESLPIEVMVDAVH |
| | | KLKYENYTSSFFIRDIIKPDPPKNLQLKP |
| | | LKNSRQVEVSWEYPDTWSTPHSYFSL |
| | | TFCVQVQGKSKREKKDRVFTDKTSATV |
| | | ICRKNASISVRAQDRYYSSSWSEWASV |
| | | PCSGSENLYFQSHHHHHHGS |

### EXAMPLES

### Materials and Methods

### Constructs

Human interleukin cDNAs were obtained from Origene (Rockville) and subsequently cloned into the pSVL vector (Amersham Biosciences). Amino acid sequences of IL-12α (SEQ ID NO: 3), IL-12β (SEQ ID NO: 4), IL-27a (SEQ ID NO: 5), and EBI3 (SEQ ID NO: 6) correspond to the UniProt accession numbers P29459, P29460, Q8NEV9, and Q14213, respectively. Where indicated, constructs were C-terminally tagged with an HA or FLAG epitope-tag, separated by a (GS)₂- or (GS)₄-linker, or equipped with a KDEL-sequence, separated by a (GS)₃-linker. Mutants were generated by site-directed mutagenesis. For mammalian protein purification IL-12α^{C96S} (SEQ ID NO: 7) (C-terminal TEV cleavage site followed by a GG-linker and His-tag), IL-12β^{C199S} (SEQ ID NO: 8) (untagged), and IL-27α^{L162C} (SEQ ID NO: 9) (C-terminal HRV-3C Protease cleavage site followed by a (GG)₂-linker and His-tag) were cloned into the pcDNA3.4TOPO vector (Gibco) and EBI3 (untagged) into the pHEK293 ultra expression vector 1 (TAKARA). All constructs were sequenced.

### Cell Culture and Transient Transfections

Mammalian cell experiments were performed in HEK293T cells, which were cultivated in Dulbecco's Modified Eagle Medium (DMEM) containing L-alanyl-L-glutamine (AQmedia, Sigma-Aldrich) at 37° C. and 5% CO₂. The medium was supplemented with 10% (v/v) FBS (Gibco) and 1% (v/v) antibiotic-antimycotic solution (25 μg/ml amphotericin B, 10 mg/ml streptomycin, 10,000 units of penicillin, Sigma-Aldrich). Transient transfections were performed in poly-D-lysine coated 6-wells (Corning) using GeneCellin (BioCellChallenge) according to manufacturer's protocol. The total transfected DNA amount was 2 μg with a DNA ratio α-subunit to β-subunit of 2:1 in case of IL-35 and IL-27 and 1:1 in case of IL-12. For subunit titration experiments either 1 μg α-subunit or 1 μg β-subunit were transfected, while increasing the corresponding other subunit in 0.5 μg steps. If only one subunit was transfected, empty pSVL vector was co-transfected to maintain a total amount of 2 μg DNA.

### Secretion Experiments

Cells were transfected for 8 h, washed twice with PBS (Sigma-Aldrich) and cultivated in 0.5 ml complete DMEM for another 16 h. To analyze secreted proteins, the medium was centrifuged for 5 min at 300 g and 4° C. The supernatant was transferred into a new reaction tube, supplemented with 0.1 volumes of 500 mM Tris/HCl (pH 7.5), 1.5 M NaCl, complemented with 10× protease inhibitor (Roche complete Protease Inhibitor w/o EDTA; Roche Diagnostics) and again centrifuged for 15 min at 20,000 g and 4° C. For cell lysis, cells were washed twice with ice-cold PBS and subsequently 0.5 ml of 1×RIPA lysis buffer (50 mM Tris/HCl, pH 7.5, 150 mM NaCl, 1% NP40, 0.5% DOC, 0.1% SDS) supplemented with 1× protease inhibitor were added to each well. After 20 min, cells were scraped off and centrifuged for 15 min at 20,000 g and 4° C. For further analysis, 0.2 volumes of 5× Laemmli buffer supplemented with 10% β-mercaptoethanol (β-Me) were added to the samples, which were then heated at 95° C. for 5 min.

### Immunoblots and Co-Immunoprecipitation (Co-IP) Experiments

Protein samples were separated by SDS-polyacrylamide gel electrophoresis (PAGE) on 12% SDS-PAGE gels at 100 V for 2 h and subsequently transferred to polyvinylidene difluoride (PVDF) membranes by blotting overnight (o/n) at 30 V and 4° C. Thereafter, membranes were blocked for 3 h at RT with Tris buffered saline containing skim milk powder and Tween-20 (MTBST; 25 mM Tris/HCl, pH 7.5, 150 mM NaCl, 5% (w/v) skim milk powder, 0.05% (v/v) Tween-20). Binding of the primary antibody was carried out o/n at 4° C. with anti-IL-12α (Abcam, ab133751; 1:500 in MTBST), anti-IL-12β(Abcam, ab133752; 1:500 in MTBST), EBI3 antisera (generously provided by O. Devergne; 1:20 in PBS), anti-IL-27 (R&D Systems, AF2526; 1:200 in MTBST), anti-HA tag (BioLegend, 902301; 1:1,000 in MTBST), anti-Hsc70 (Santa Cruz Biotechnology, sc-1059; 1:1,000 in MTBST) or anti-His tag (Proteintech, HRP-66005; 1:1,000 in MTBST). After washing, membranes were incubated for 1 h in species-specific HRP-conjugated secondary antibodies (Santa Cruz Biotechnology; 1:10,000 in MTBST). Immunoblots were detected with Amersham ECL prime (Cytiva) and a Fusion Pulse 6 imager (Vilber Lourmat).

For co-immunoprecipitation experiments, cells were lysed after washing with PBS in 0.5 ml Triton lysis buffer (50 mM Tris/HCl, pH 7.5, 150 mM NaCl, 1 mM EDTA, 1% Triton X-100, 1× protease inhibitor) and cleared by centrifugation at 20,000 g for 15 min and 4° C. To analyze secreted proteins, the medium was treated as described above, then pre-cleared by the addition of 30 μl Protein A/G agarose (Santa Cruz Biotechnology) and rotation for 1 h at 4° C. Subsequently, all samples were incubated with 25 μl target-specific magnetic beads (anti-FLAG M2 (Sigma-Aldrich, M8823) or anti-HA (Thermo Fisher Scientific, 88837), while rotating for 2 h at 4° C. Beads were washed three times with NP40 wash buffer (50 mM Tris/HCl, pH 7.5, 400 mM NaCl, 0.5% NP40, 0.5% DOC). By adding 2× Laemmli buffer containing 4% β-Me and heating for 5 min at 95° C., proteins were eluted and then separated by SDS-PAGE.

For co-immunoprecipitation experiments with purified proteins 1 μg IL-12α^{C96S,His} (SEQ ID NO: 32) and an equimolar amount of IL-12β^{C199S} (SEQ ID NO: 8) (previously purified in our lab), 1 μg EBI3 (SEQ ID NO: 6) and equimolar mIL-27α^{His} (SEQ ID NO: 33) (R&D Systems 7430-ML-010), or 1 mg IL-12α^{C96S,His} (SEQ ID NO: 32) and equimolar EBI3 (SEQ ID NO: 6) were mixed to a final volume of 200 μl (PBS) and incubated for 1 h at RT. 30 μl NiNTA beads (Sigma-Aldrich) were added to the solution and incubated while rotating for 2 h at 4° C. Beads were washed with PBS containing 20 mM imidazole and elution was performed by addition of PBS supplemented with 500 mM imidazole and incubation for 10 min at 4° C. For further analysis by SDS-PAGE, 0.2 volumes of 5× Laemmli buffer supplemented with 10% β-Me were added to the supernatants, which were then heated at 95° C. for 5 min.

### Mammalian Protein Production and Purification

For expression of IL-12α^{C96S,His} (SEQ ID NO: 32), ExpiCHO cells (Thermo Fisher Scientific) were co-transfected with IL-12α^{C96S,His} (SEQ ID NO: 32) and IL12β^{C199S} (SEQ ID NO: 8) in a DNA ratio of 1:1 according to manufacturer's protocol (high titer). After protein expression for 7 days, the medium was centrifuged (5,000 g, 30 min, 4° C.) and applied to a HisTrap HP column (Cytiva). A guanidinium chloride gradient (final 2.5 M GdnCl) was performed to separate IL12β^{C199S} (SEQ ID NO: 8) from IL-12α^{C96S,His} (SEQ ID NO: 32), subsequent washing with PBS on column was applied to refold IL-12α^{C96S,His} (SEQ ID NO: 32). Elution was performed in PBS supplemented with 500 mM imidazole and the His-tag was optionally cleaved by addition of TEV protease (TEV: IL-12α^{C96S} 1:10 (w/w)) o/n at 4° C. and removed by a second HisTrap HP column in PBS. Final purification was performed using a HiLoad 26/600 Superdex 200 μg column (Cytiva) in PBS. EBI3 (SEQ ID NO: 6) was cotransfected with IL27α^{L162C,His} (SEQ ID NO: 34) into Expi293 (Thermo Fisher Scientific) cells with a DNA ratio of 1:1 according to manufacturer's protocol. After 2 days the medium was centrifuged and applied to a HisTrap column. To separate the complex, a guanidinium chloride wash gradient (final 6 M GdnCl) was performed to elute EBI3 (SEQ ID NO: 6) from the column, which was further purified by a HiPrep 16/60 Sephacryl S-200 HR with PBS and 3 M GdnCl. EBI3 (SEQ ID NO: 6) containing fractions were pooled, concentrated, and dialyzed against PBS.

### Far-UV Circular Dichroism (CD) Spectroscopy

Measurements were performed on a J-1500 CD spectrometer (Jasco) using a 10 μM protein solution in PBS in a quartz cuvette with 1 mm pathlength. Spectra were recorded from 200-260 nm at 20° C., temperature transitions from 20-90° C. at 222 nm for IL-12α^{C69S} (SEQ ID NO: 7), and 218 nm for EBI3 (SEQ ID NO: 6) with a heating rate of 30° C./h.

### Hydrogen Deuterium Exchange (HDX) Mass Spectrometry (MS)

HDX measurements were performed following the protocols established in (56), using a ACQUITY UPLC M-class system equipped with automated HDX technology followed by an in-line Synapt G2-S QTOF HDMS mass spectrometer (Waters). In short, HDX kinetics were recorded for biological duplicates as technical triplicates tracking data points at 0 s, 10 s, 1 min, 10 min, 30 min, and 2 h. At each data point, 3 μl of a 20 μM protein solution containing both proteins in molar 1:1 ratio were diluted automatically 1:20 with PBS buffer (pH 7.4) containing 99.9% D₂O or H₂O at each time point. The exchange was stopped by addition of 1:1 quenching buffer (200 mM Na₂HPO₄, 200 mM KH₂PO₄, pH 2.3, containing 4 M GdmCl and 200 mM TCEP) at 1° C.

Proteolytic on-column digestion was performed on a Waters Enzymate BEH Pepsin Column (2.1×30 mm) at 20° C. The resulting peptides were separated by reverse phase chromatography at 0° C. using a Waters Acquity UPLC C18 1.7 μm Vangard 2.1×5 mm trapping-column and a Waters Aquity UPLC BEH C18 1.7 μm 1×100 mm separation column applying an H₂O to acetonitrile gradient with both eluents containing 0.1% formic acid (v/v) to allow deuterium replacement with hydrogen from side chains and N-/C-termini that exchange faster than backbone amide linkages. MS data of eluting peptides were collected over an m/z range of 100-2000 using Glu-fibrino peptide B (Waters) to ensure mass accuracy. Peptides were identified by MS^{E} ramping the collision energy automatically from 20-50 V. Due to the use of an automated system deuterium levels were not corrected for back exchange and are reported as relative levels. Data analysis was performed with the PLGS (version 3.0.3) and DynamX (version 3.0) software packages (Waters).

### Analytical Ultracentrifugation

Sedimentation velocity analytical ultracentrifugation (SV-AUC) experiments were performed on a Beckman Coulter Optima™ AUC analytical ultracentrifuge (Beckman Coulter) equipped with absorbance optics. For each sample, 350 μl of 10 μM IL-12α^{C96S} (SEQ ID NO: 7) and EBI3 (SEQ ID NO: 6) or the equimolar incubated proteins in PBS, pH 7.4, were loaded into a standard 12 mm double-sector epon-filled centerpiece, covered with quartz windows, alongside with 450 μl of the reference buffer solution. Samples were centrifuged at 42,000 rpm using an An-50 Ti rotor at 20° C. (with an initial test run at 3,000 rpm). Radial absorbance scans were acquired continuously at 235 nm with a radial step size of 0.001 cm. The obtained sedimentation velocity profiles were analyzed using SEDFIT software with a non-model based continuous Svedberg distribution method (c(s)), with time (TI) and radial (RI) invariant noise on (57). The density (p) and viscosity (q) of PBS used for data analysis was experimentally determined.

### NanoBRET Assay

Receptor chains were cloned into the pHTC HaloTag® CMV-neo Vector (IL-12Rβ2, gp130) or the pNLF1-C [CMV/Hygro] Vector (IL-12Rβ2, IL-27Rα) (Promega). COS7 cells were cultivated under the same conditions as described for HEK293T cells and transient transfections were performed in uncoated 6-well plates using GeneCellin according to manufacturer's protocol. In total, 2 μg DNA were transfected per well with a ratio of 100:1 HT:NL. After 16 h, transfected cells were detached via Accutase (Sigma-Aldrich), divided into two pools and 1 μl HaloTag® NanoLuc® 618 Ligand (Promega) or 1 μl DMSO as a control per ml cells were added. 2×10⁴ cells were seeded into white bottom 96-wells and incubated for another 20 h. Reconstituted proteins were incubated for 1 h at RT and cytokines were added 30 min at a final concentration of 10 nM before measurement on a plate reader (BMG Labtech CLARIOstar®). For inhibition experiments 10, 100, or 1000 nM IL-12α^{C96S} (SEQ ID NO: 7) or EBI3 (SEQ ID NO: 6) were added 30 min before stimulation with IL-12 or IL-27. Mean milliBRET units (mBU) were calculated by dividing the acceptor emission by the donor emission and multiplication with 1,000. To determine the mean NanoBRET® ratio, the no-acceptor control mean is subtracted from the experimental mean. Samples are measured in technical triplicates and one representative result from at least biological triplicates is shown.

### STAT Assays

BL-2 cells were cultivated in RPMI-1640 (ATCC modification, Thermo Fisher Scientific) supplemented with 20% heat inactivated FBS (Gibco) and 1% (v/v) antibiotic-antimycotic solution (25 μg/ml amphotericin B, 10 mg/ml streptomycin, 10,000 units of penicillin; Sigma-Aldrich). Cells were starved o/n in RPMI-1640 without FBS and antibiotic-antimycotic solution and 1-2×10⁶ cells in RPMI-1640+0.5% (w/v) bovine serum albumin (Sigma-Aldrich) were then seeded into 48-wells and incubated for 15 min at 37° C. Reconstituted proteins were incubated for 1 h at RT and BL-2 cells were stimulated with cytokines for 1 h at a final concentration of 10 ng/ml. For inhibition experiments, 10, 100 or 1000 ng/ml EBI3 (SEQ ID NO: 6) were added 30 min before stimulation with IL-27. O/n treatment was performed with 10 ng/ml EBI3 (SEQ ID NO: 6) and additionally 10 ng/ml EBI3 (SEQ ID NO: 6) were added 30 min before IL-27 stimulation. 600 μl ice-cold PBS+0.05% NaN₃ were added to each well to stop the reaction and the cells were transferred into a reaction tube, which was then centrifuged at 300 g for 5 min at 4° C. Supernatant was discarded and cells were lysed by addition of 100 μl NP40 buffer supplemented with 1× protease inhibitor and phosphatase inhibitor (Serva) and incubated for 20 min, 4° C. while rotating. After centrifugation for 5 min at 20,000 g, and 4° C., 0.2 volumes of 5× Laemmli buffer supplemented with 10% β-Me were added to the supernatant, boiled for 5 min and further analyzed via immunoblot.

NK-92 cells were cultivated at 37° C., 5% CO₂ in Minimum Essential Medium (MEM) α (Sigma-Aldrich), containing 2.2 g/L NaHCO₃. The medium was further supplemented with 0.2 mM Myoinositol (Sigma-Aldrich, F-7508), 0.1 mM β-Me, 0.02 mM folic acid (Sigma-Aldrich, F-8758), 12.5% FBS (Gibco), 12.5% horse serum (Thermo Fisher Scientific, 16050122), and 100 U/ml freshly added IL-12 (Peprotech, 200-02). NK-92 cells were starved o/n in medium without serum and IL-2. 0.5-1×10⁶ cells were seeded into 48-wells and incubated at 37° C. for 15 min before stimulation with cytokines (reconstituted proteins were previously incubated for 1 h at RT). Stimulation was performed for 30 min at a final concentration of 10 ng/ml. For inhibition experiments, 10, 100 or 1000 ng/ml IL-12α^{C96S} (SEQ ID NO: 7) were added 30 min before stimulation with IL-12. O/n treatment was performed with 10 ng/ml IL-12α^{C96S} (SEQ ID NO: 7) and additionally 10 ng/ml IL-12α^{C69S} (SEQ ID NO: 7) were added 30 min before IL-12 stimulation. Cells were harvested on ice, transferred into a reaction tube and centrifuged at 300 g for 5 min at 4° C. The cell pellet was reconstituted in 100 μl RIPA lysis buffer supplemented with 1× protease and phosphatase inhibitor and lysis was performed for 20 min at 4° C. while rotating. Cell debris was removed by centrifugation at 20,000 g, 5 min at 4° C. and the supernatant was supplemented with 0.2 volumes of 5× Laemmli buffer supplemented with 10% β-Me, heated for 5 min at 95° C. and further analyzed via immunoblots.

### PBMC and MDM Culture and Flow Cytometry Analysis

Peripheral blood mononuclear cells (PBMCs) of healthy individuals were isolated. The fraction after CD14 MACS separation was directly resuspended in RPMI-1640 (Thermo Fisher Scientific) supplemented with 10% FBS (BioSell), 1 μg/ml Gentamycin (Thermo Fisher Scientific), 100 U/ml Penicillin-Streptomycin (Thermo Fisher Scientific) and 2 mM L-Glutamine (Thermo Fisher Scientific). 1×10⁶ cells were seeded into 24-wells and stimulated for 24 h with 100 ng/ml LPS (Invivogen) and either with 10 ng/ml EBI3 (SEQ ID NO: 6) or 10 ng/ml IL-12α^{C96S}(SEQ ID NO: 7). The CD14⁺ fraction was used to generate monocyte-derived macrophages (MDM) as previously reported^{67,68}. 0.5×10⁶ cells/ml were cultured in RPMI-1640 (Thermo Fisher Scientific) supplemented with 10% FBS (BioSell), 1 μg/ml Gentamycin (Thermo Fisher Scientific), 100 U/ml Penicillin-Streptomycin (Thermo Fisher Scientific), 2 mM L-Glutamine (Thermo Fisher Scientific), 10 ng/mL human GM-CSF (Miltenyi) and 2 ng/ml human TGFβ (Miltenyi) for 6 days at 37° C. with 5% CO₂ to differentiate the cells into alveolar-like macrophages⁴⁸. After 6 days incubation MDM were harvested, 1-2×10⁵ cells were seeded into 96-wells and stimulated for 24 h with 10 μg/ml house dust mite extract (HDM) (Citeq Biologics) and either with 10 ng/ml EBI3 (SEQ ID NO: 6) or 10 ng/ml IL12α^{C96S} (SEQ ID NO: 7). During harvest, supernatants were stored at −70° C. until further cytokine analysis and cells were lysed in RLT buffer (Qiagen) supplemented with 1% β-Me and stored at −70° C. until RNA isolation.

For Treg induction and characterization, 2×10⁵ PBMCs per well were resuspended in RPMI-1640 medium (Thermo Fisher Scientific) supplemented with 10% heat-inactivated and filtered FCS (Sigma-Aldrich) and 1% penicillin/streptomycin (Thermo Fisher Scientific) and incubated with 10 ng/ml EBI3 (SEQ ID NO: 6), IL-12α^{C96S} (SEQ ID NO: 7) or PBS as control for 72 h at 37° C. in a 5% CO₂ atmosphere. Treg induction was characterized as CD3⁺CD4⁺CD127⁻CD25^{hi}FoxP3⁺ cells by FACS as previously described⁶⁹ with the following anti-human antibodies and clones: CD3 (clone UCHT1), CD4 (clone RPA-T4), CD127 (clone A019D5), CD25 (clone BC96) (all from BioLegend) and FoxP3 (clone PCH101) (Invitrogen). For SEA stimulation, 2×10⁵ PBMCs were left untreated (PBS control) or cultured with 50 μg/ml of SEA, prepared from S. mansoni eggs as previously detailed⁷⁰, alone or in combination with either 10 ng/ml EBI3 (SEQ ID NO: 6) or IL-12α^{C96S} (SEQ ID NO: 7) for 5 days. On day 3, 50% of culture medium were exchanged with fresh medium containing respective stimuli (SEA±EBI3 or IL-12^{C96S}) or PBS as control. Culture supernatants were collected and IL-4 concentrations were determined by ELISA.

### Cytokine Analysis (ELISA)

PMBC and MDM supernatants were analyzed for IL-4 using the IL-4 human ELISA kit from Thermofisher Scientific (KH00041), and IL-6, IL-1β, and IL-8 secretion using the human ELISA Sets (BD Biosciences, 555220, 557953, 555244). TNFα secretion was analyzed in supernatants using the human DuoSet ELISA (R&D Systems, DY210). All ELISAs were performed according to the manufacturer's instructions.

### RNA Isolation

RNA was extracted using a spin-column kit according to the manufacturer's instructions (Zymo Research) and transcribed into DNA using the HighCapacity cDNA Reverse Transcription kit according to the manufacturer's instructions (Applied Biosystems).

RT qPCR

10 ng cDNA were used as a template and primers were mixed with FastStart Universal SYBR Green Master Mix (Roche). Fluorescence was measured on a ViiA7TM Real-Time PCR System (Applied Biosystems, Thermo Fisher Scientific). The expression levels were normalized to the house-keeping genes GAPDH (for MDM), ACTB and 18S (for PBMCs). Relative gene expression was calculated as 2ΔCT (ΔCT=CT (Housekeeper)−CT(Gene)).

TABLE 2
Primer sequences used for qPCR:

| Human | | |
|---|---|---|
| Primer | Forward sequence | Reverse sequence |
| 18S | GTAACCCGTTGAACCCCATT | CCATCCATTCGGTAGTAGCG |
| | (SEQ ID NO: 10) | (SEQ ID NO: 11) |
| ACTB | GGATGCAGAAGGAGATCACT | CGATCCACACGGAGTACTTG |
| | (SEQ ID NO: 12) | (SEQ ID NO: 13) |
| CXCL8 | GAAGTTTTTGAAGAGGGCTG | TGCTTGAAGTTTCACTGGC |
| | AGA (SEQ ID NO: 14) | AT (SEQ ID NO: 15) |
| GAPDH | GAAGGTGAAGGTCGGAGT | GAAGATGGTGATGGGATTTC |
| | (SEQ ID NO: 16) | (SEQ ID NO: 17) |
| IL1B | AGAAGTACCTGAGCTCGCCA | CTGGAAGGAGCACTTCATCT |
| | (SEQ ID NO: 18) | GT (SEQ ID NO: 19) |
| IL6 | ACATGTGTGAAAGCAGCAAAG | GGCAAGTCTCCTCATTGAAT |
| | (SEQ ID NO: 20) | CC (SEQ ID NO: 21) |
| TNF | CCCATGTTGTAGCAAACCCTC | TATCTCTCAGCTCCACGCCA |
| | (SEQ ID NO: 22) | (SEQ ID NO: 23) |
| TGFB | CACGCAGTACAGCAAGGTCC | CCACGTAGTACACGATGGGC |
| | (SEQ ID NO: 24) | (SEQ ID NO: 25) |
| IL5 | TCTCCAGTGTGCCTATTCCC | CGAACTCTGCTGATAGCCAA |
| | (SEQ ID NO: 26) | (SEQ ID NO: 27) |

### Quantification and Statistics

Immunoblots were quantified using the Bio-1 D software (Vilber Lourmat). Statistical analyses were performed using Prism (GraphPad Software). Differences were considered statistically significant when p<0.05. Where no statistical data are shown, all experiments were performed at least three times, with one representative experiment depicted in figures. For immunological data, Friedmann test was used and p<0.05 was considered statistically significant. Details of statistical tests and sample sizes are provided in the figure legends.

### Structural Modeling

The inventors generated a model-structure for the IL-35 sequence with the alphafold multimer module^{71,72}. The sequences of human IL12α (SEQ ID NO: 3) and EBI3 (SEQ ID NO: 2) without the signal-peptide were used as input, and the best ranked structure from the output was taken as the final model-structure.

### Example 1: IL-12α and EBI3 Mutually Promote their Secretion in Different Assembly States

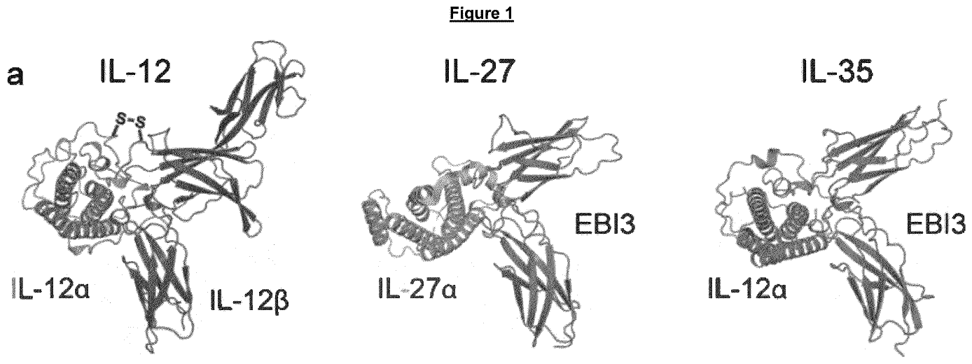

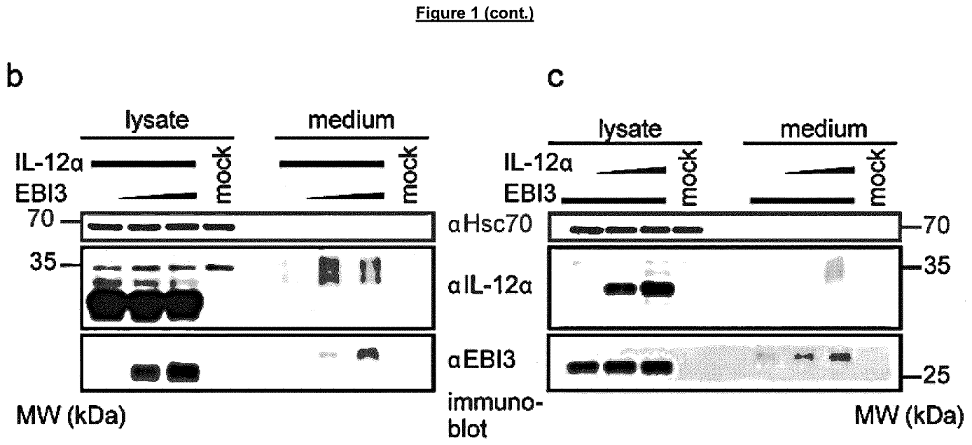

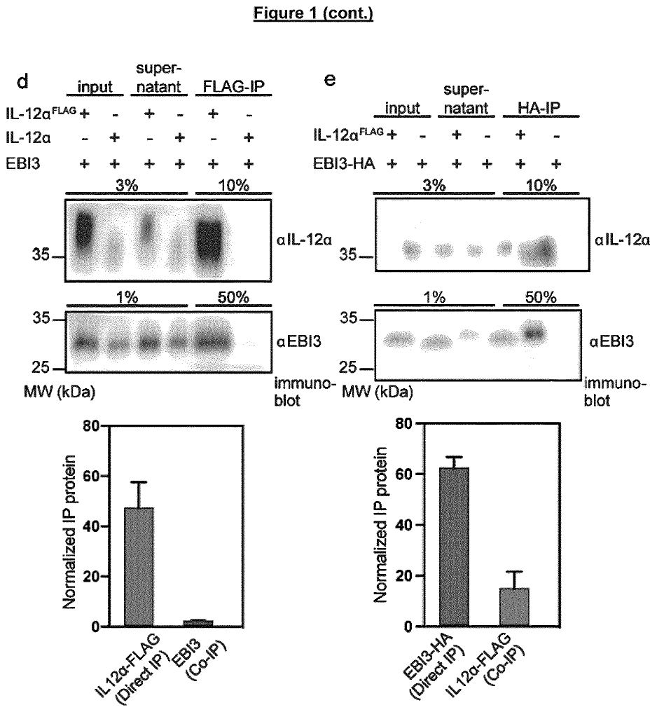

Since its first description³⁶ and functional characterization⁸, IL-35 has remained a structurally ill-defined cytokine. This has hampered progress in understanding this potent immunosuppressive molecule as well as in potentially using or targeting it in the clinics. IL-35 is composed of IL-12α/p35 and EBI3, subunits that are shared with IL-12 or IL-27, respectively (see FIG. 1a).

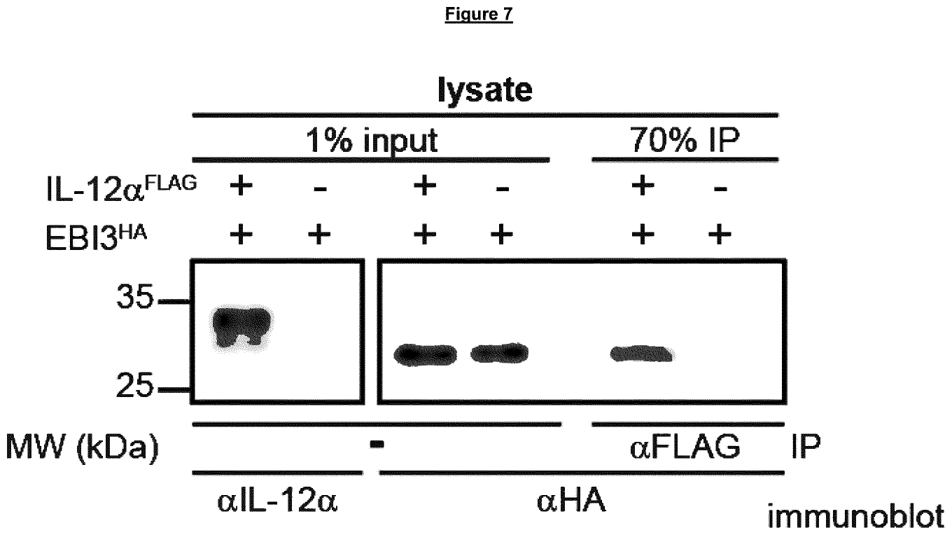

In agreement with a productive interaction of IL-12α (SEQ ID NO: 3) and EBI3 (SEQ ID NO: 6), IL-12α (SEQ ID NO: 3) was hardly secreted in isolation, but required the presence of EBI3 (SEQ ID NO: 6) to induce its secretion (see FIG. 1b). Vice versa, IL-12α (SEQ ID NO: 3) slightly increased EBI3 (SEQ ID NO: 6) secretion (see FIG. 1c). This dependency hints towards complex-formation between both subunits. Indeed, in accordance with the first report on IL-35³⁶, the inventors of the present invention were able to detect interaction of both subunits in co-immunoprecipitation experiments in cell lysates (see FIG. 7) and when secreted into the cell medium (see FIGS. 1d and 1e). However, the data produced by the inventors of the present invention revealed that only 2% of the overall secreted EBI3 (SEQ ID NO: 6) pool co-IPed with IL-12α (SEQ ID NO: 3) FLAG (where the IP efficiency was 48%, see FIG. 1d). Vice versa, only 15% of the secreted IL-12α (SEQ ID NO: 3) pool co-IPed with secreted EBI3-HA (where the IP efficiency was 63%, see FIG. 1e). Assuming an immunoprecipitation efficiency of 100%, the data produced by the inventors of the present invention show that when co-expressed only approximately 4% of secreted EBI3 (SEQ ID NO: 6) were bound to IL-12α (SEQ ID NO: 3) and vice versa, only approximately 24% of secreted IL-12a (SEQ ID NO: 3) were complexed with EBI3 (SEQ ID NO: 6). Thus, at a first glance, IL-35 shares key features of other IL-12 family members: assembly-induced secretion of the subunits and their interaction, albeit weak³⁶⁻⁴⁰. However, in contrast to other IL-12 family members, by far most of the secreted subunits do not seem to be part of heterodimeric IL-35.

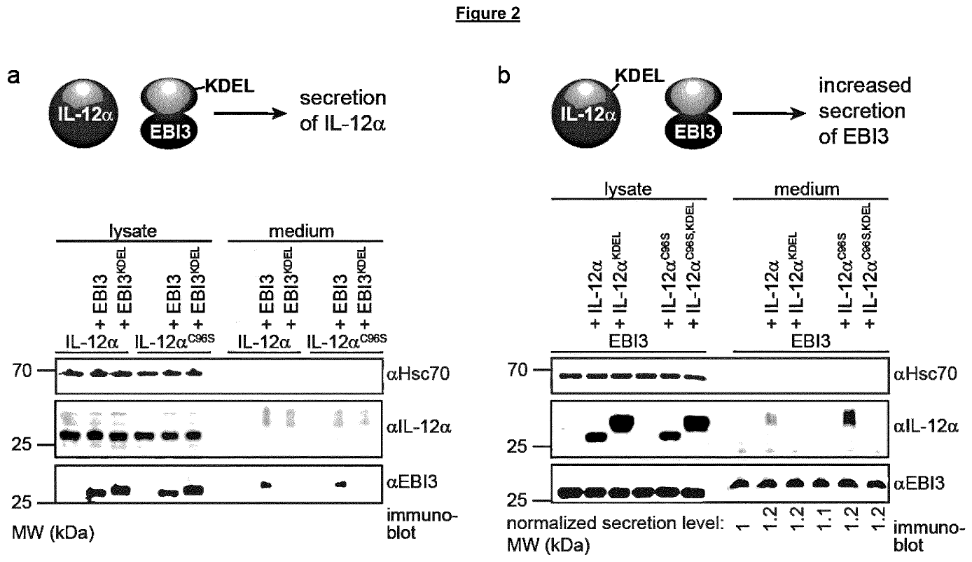

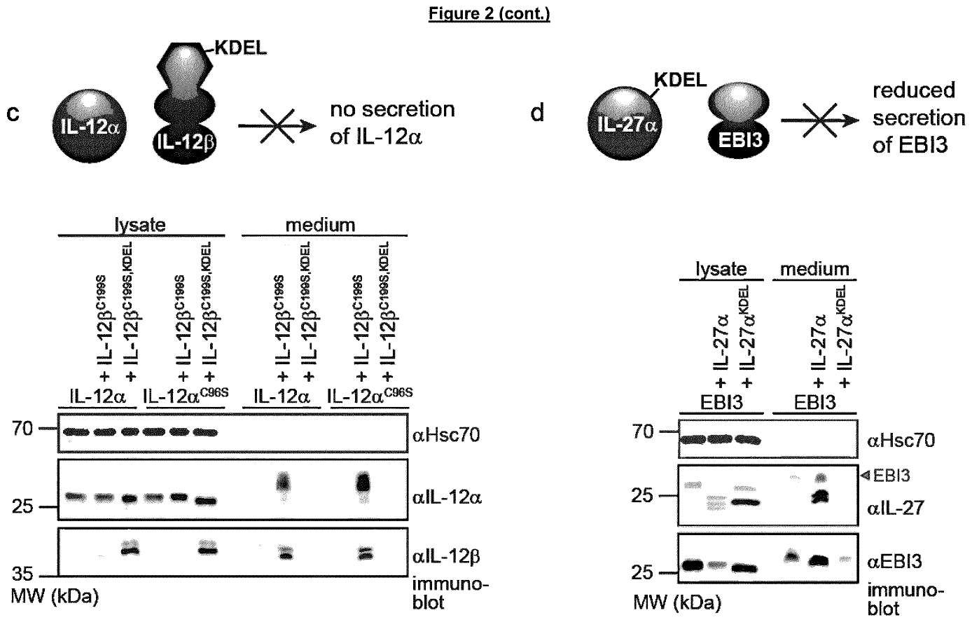

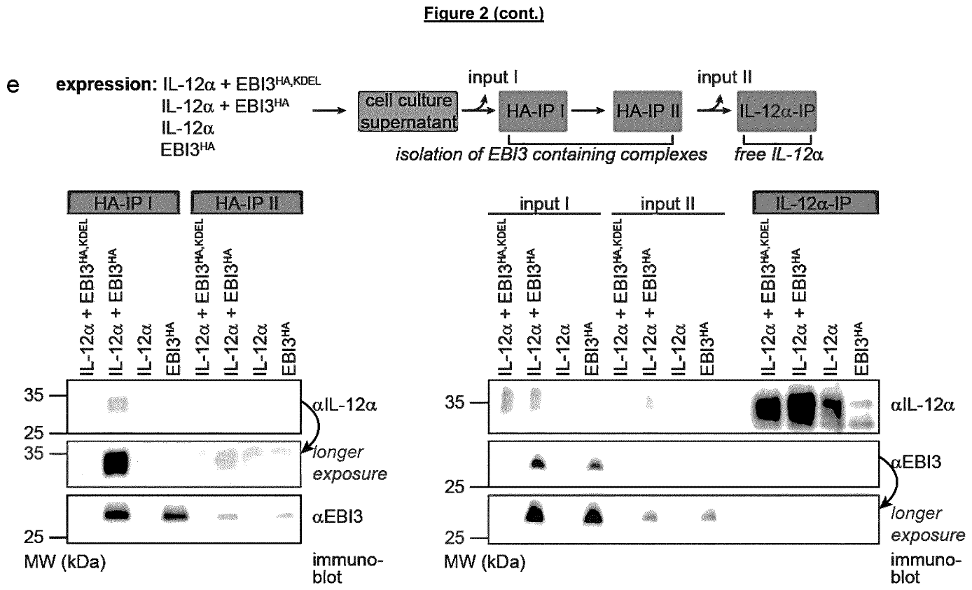

This prompted the inventors of the present invention to more thoroughly investigate IL-12α (SEQ ID NO: 3) and EBI3 (SEQ ID NO: 6) secretion upon co-expression. These analyses revealed a fundamentally different behavior of IL-35 in comparison to other IL-12 family members. When either IL-12α (SEQ ID NO: 3) or EBI3 (SEQ ID NO: 6) were furnished with a C-terminal KDEL sequence (IL-12α^{KDEL} (SEQ ID NO: 28) or EBI3^{KDEL} (SEQ ID NO: 29), respectively), which leads to ER retention of the respective protein⁴¹, these subunits were retained in cells (see FIGS. 2a and 2b), as expected. Quite unexpectedly, though, EBI3^{KDEL} (SEQ ID NO: 29) could still induce secretion of IL-12α (SEQ ID NO: 3) (see FIG. 2a). Furthermore, levels of secreted IL-12α (SEQ ID NO: 3) appeared relatively independent of the co-secretion of EBI3 (SEQ ID NO: 6) (see FIG. 2a). Likewise, IL-12α^{KDEL} (SEQ ID NO: 28) slightly increased secretion of EBI3 (SEQ ID NO: 6) over its basal levels (see FIG. 2b). These findings show that IL-12α (SEQ ID NO: 3) and EBI3 (SEQ ID NO: 6) mutually promote their secretion without necessarily being secreted as a heterodimer. This behavior is in contrast to all other known pairings within the IL-12 family that share subunits with IL-35 (SEQ ID NOs: 1 and 2). For IL-12, where IL-12α (SEQ ID NO: 3) pairs with IL-12β (SEQ ID NO: 4), IL-12β^{C199S,KDEL} (SEQ ID NO: 30) did not induce the secretion of IL-12α (SEQ ID NO: 3), even when the interchain-disulfide bond that is dispensable for IL-12 secretion⁴² was deleted (C96S in IL-12α and C199S in IL-12β) (see FIG. 2c). Instead, IL-12β^{C199S,KDEL} (SEQ ID NO: 30) co-retained IL-12α in cells (see FIG. 2c). Analogously, for IL-27, where EBI3 (SEQ ID NO: 6) pairs with IL-27α (SEQ ID NO: 5), IL-27α^{KDEL} (SEQ ID NO: 31) did not increase the secretion of EBI3 (SEQ ID NO: 6), but instead reduced it and thus also co-retained it (see FIG. 2d). Furthermore, EBI3^{KDEL} (SEQ ID NO: 29) has recently been shown to co-retain IL-27α (SEQ ID NO: 5) in cells⁴³. Together, these data reveal a different behavior for IL-35 than for other IL-12 family members. IL-35 subunits may not only be secreted in an assembled state as IL-35, but also as free IL-12α (SEQ ID NO: 3) and EBI3 (SEQ ID NO: 6), which would also explain the low amount of complex formation observed in cell media (see FIGS. 1d and 1e). To further test the inventors' hypothesis of a separate IL-12α (SEQ ID NO: 3) and EBI3 (SEQ ID NO: 6) secretion in cells expressing both subunits, the inventors next examined if IL-35 subunits (SEQ ID NOs: 1 and 2) expressed without any retention sequence are detectable as free IL-12α (SEQ ID NO: 3) and EBI3 (SEQ ID NO: 6) in the medium. Indeed, in addition to assembled IL-35, the experiments of the inventors of the present invention confirmed the presence of secreted, unassembled EBI3 (see FIG. 2e). Furthermore, the inventors could detect a pool of free IL-12α (SEQ ID NO: 3) secreted from IL-12α (SEQ ID NO: 3) and EBI3 (SEQ ID NO: 6) co-expressing cells (see FIG. 2e). Thus, taken together, the data of the inventors of the present invention show that cells expressing IL-12α (SEQ ID NO: 3) and EBI3 (SEQ ID NO: 6) secrete assembled IL-35, but also an excess of unassembled IL-12α (SEQ ID NO: 3) and EBI3 (SEQ ID NO: 6).

### Example 2: IL-12α and EBI3 are Stable Proteins in Isolation

These findings show that IL-12α (SEQ ID NO:3) and EBI3 (SEQ ID NO: 6) may act as independent immune signaling molecules secreted from cells that produce both subunits. This notion has a profound impact on the understanding of the human IL-12 family and thus requests further analyses on the structure and function of isolated IL-12α (SEQ ID NO: 3) and EBI3 (SEQ ID NO: 6).

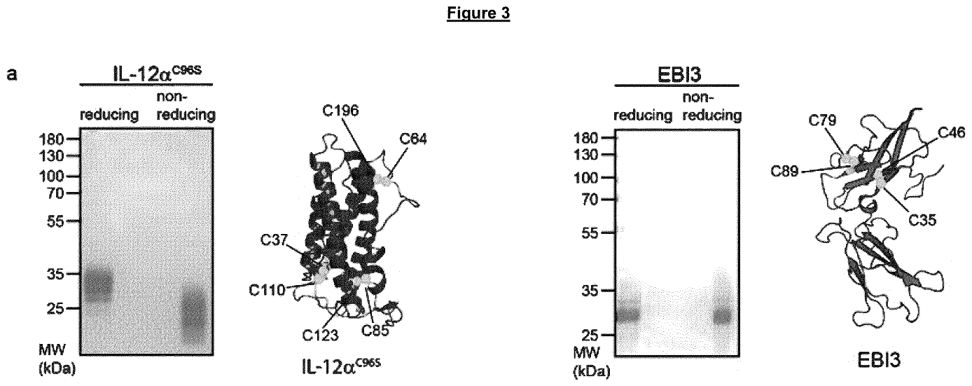

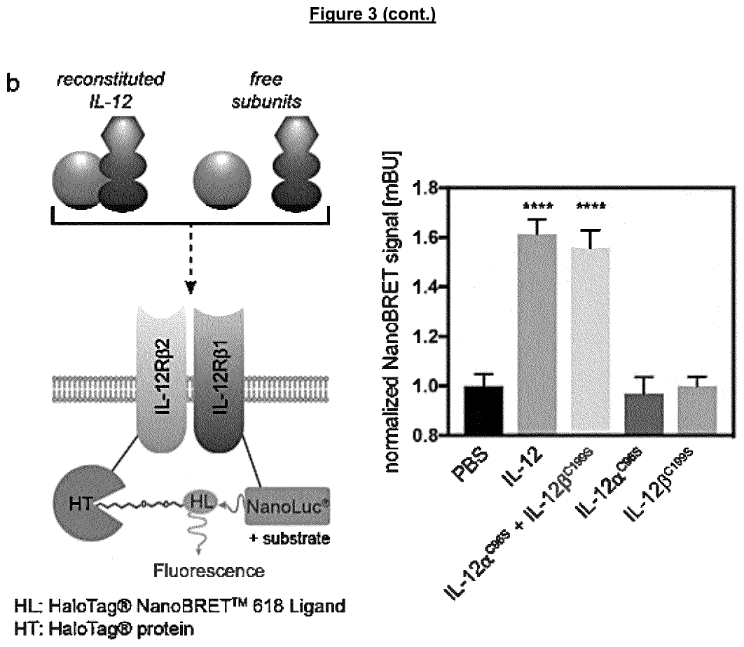

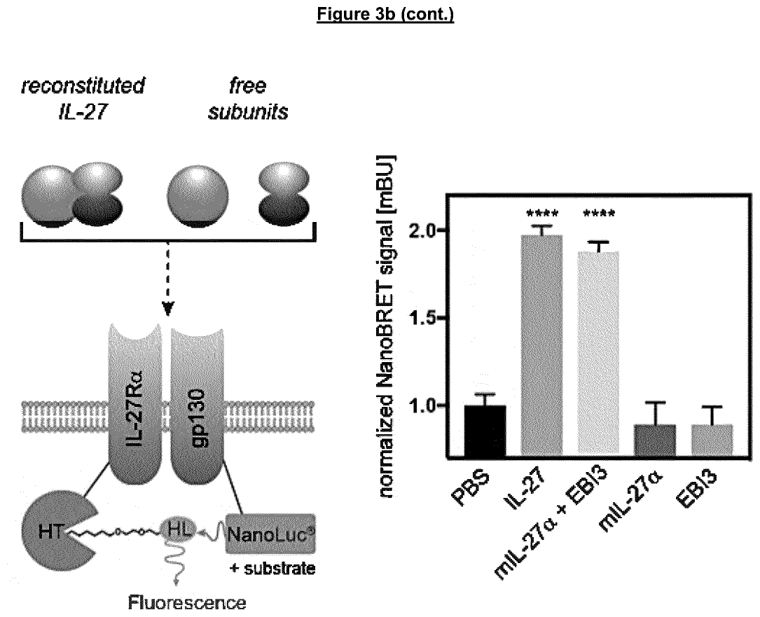

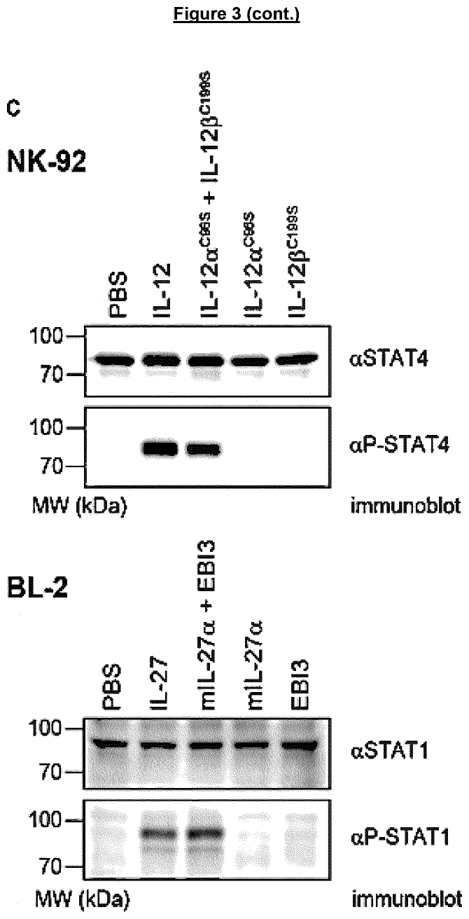

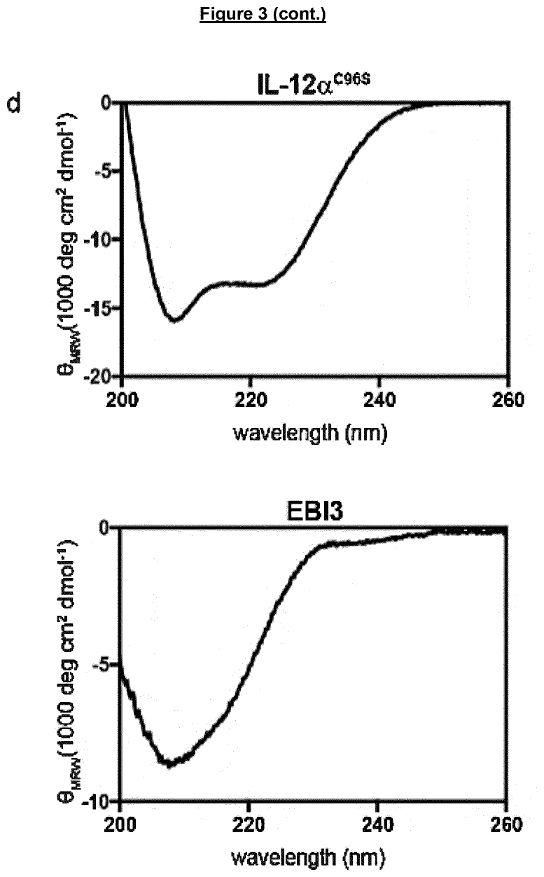

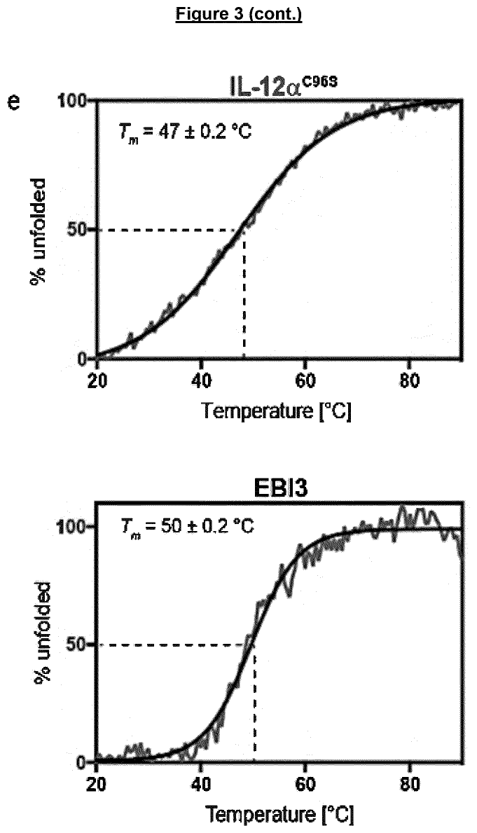

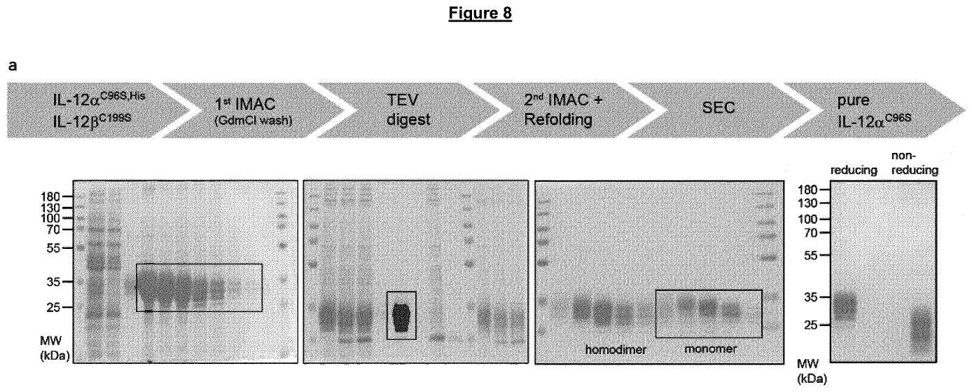

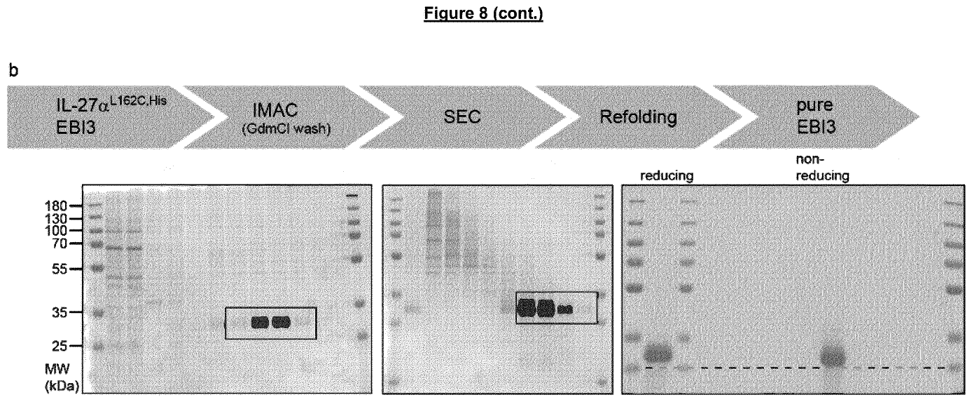

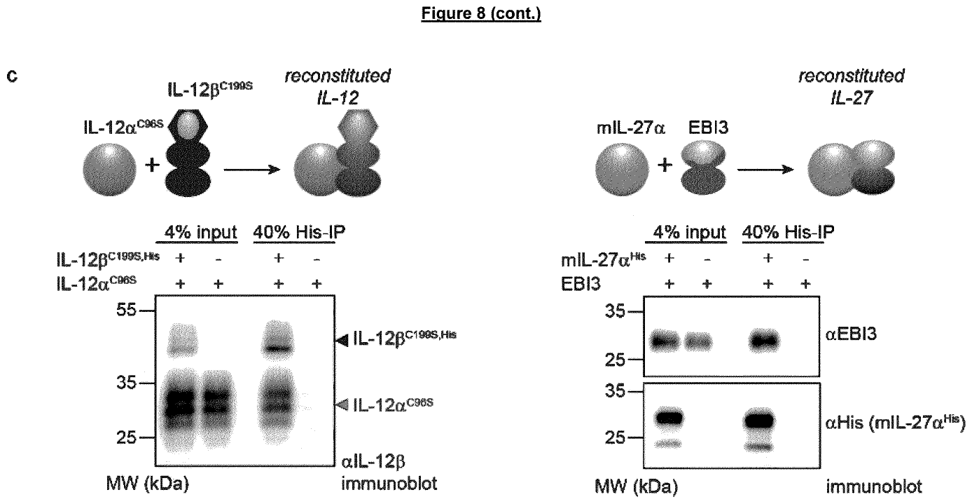

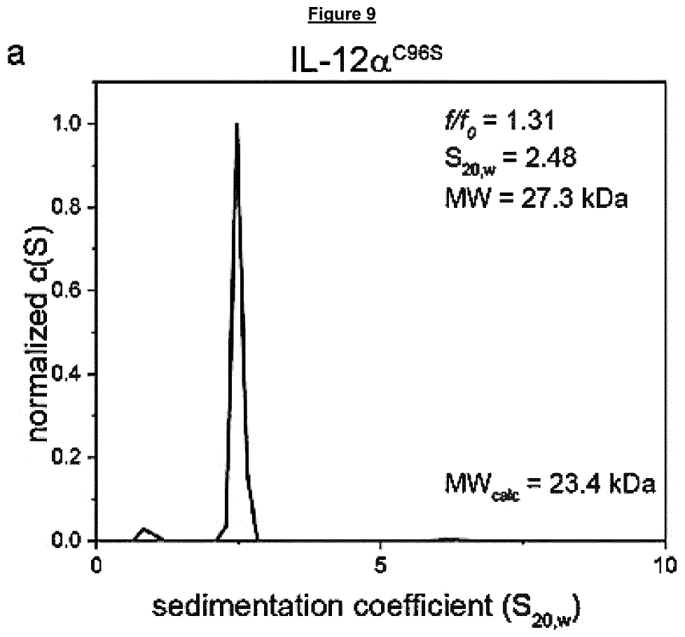

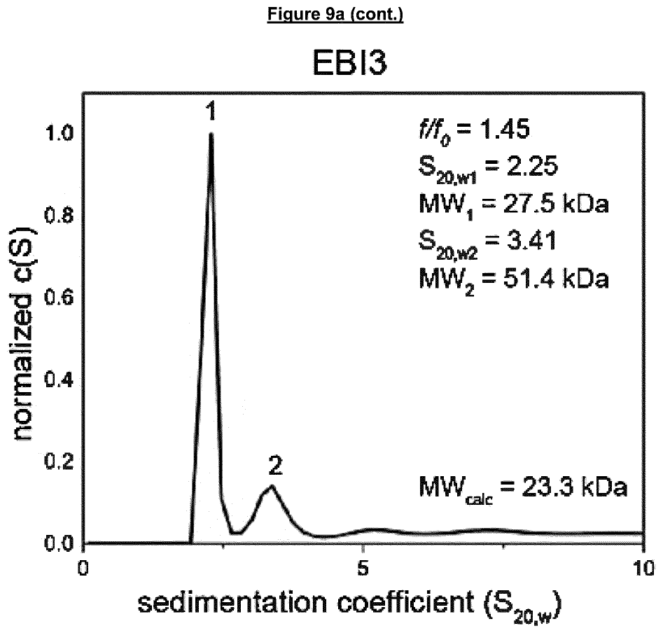

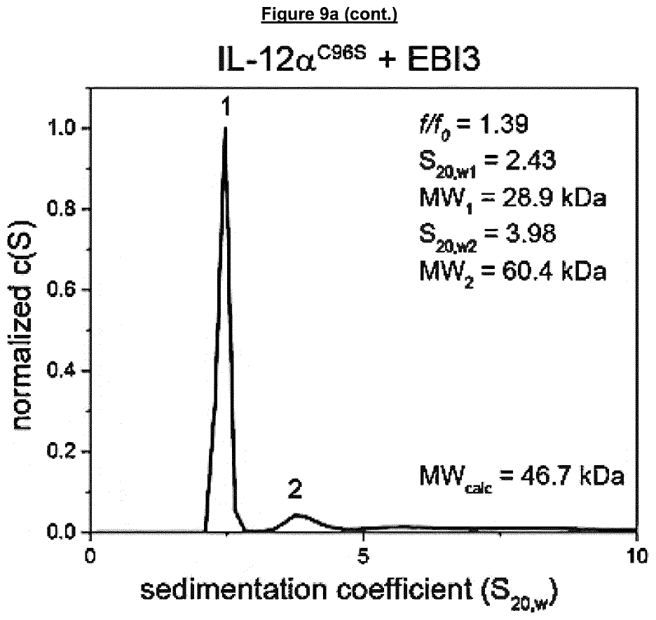

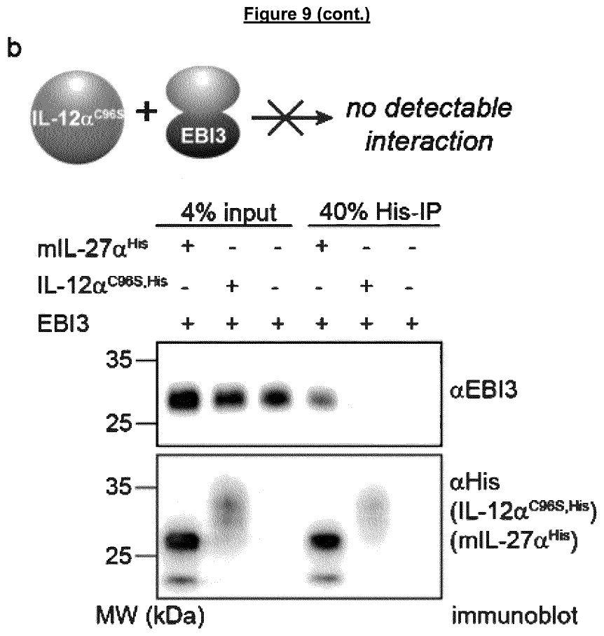

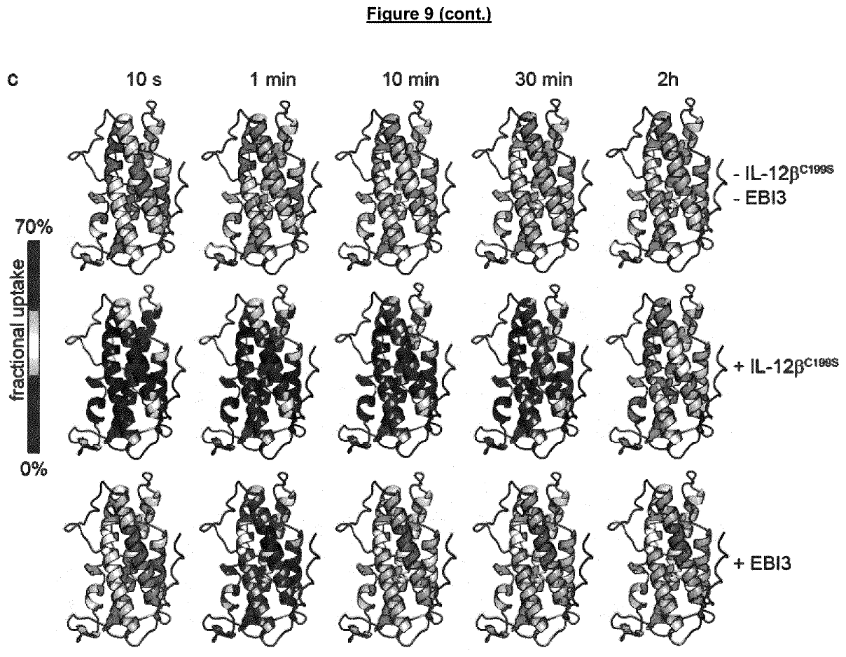

IL-12α (SEQ ID NO: 3) and EBI3 (SEQ ID NO: 6) are both disulfide-containing glycoproteins⁴⁴, which require mammalian cells for protein production to obtain authentic glycosylation patterns. IL-12α (SEQ ID NO: 3), however, is mostly retained in human cells in isolation^{37,42} and EBI3 (SEQ ID NO: 6) is poorly secreted⁴⁵ (see also FIG. 1). The inventors of the present invention thus devised strategies that allowed for the production of IL-12α (SEQ ID NO: 3) and EBI3 (SEQ ID NO: 6) in sufficient quantities from human cells. As opposed to IL-35, IL-12, which also contains IL-12α (see FIG. 1a), is secreted efficiently and can be purified from cell medium. The inventors thus developed a protocol to purify IL-12 lacking the intermolecular disulfide bridge from human cells and subsequently separated IL-12α^{C96S} (SEQ ID NO: 7) from IL-12β^{C199S} (SEQ ID NO: 8) (see FIG. 8a). Of note, C96 was dispensable for EBI3-induced secretion of IL-12α (SEQ ID NO: 3) (see FIG. 2a). The approach of the inventors of the present invention gave rise to pure and glycosylated IL-12α^{C96S} (SEQ ID NO: 7) containing its intramolecular disulfide bonds (see FIG. 3a). Analogously, to produce EBI3 (SEQ ID NO: 6), the inventors of the present invention developed a strategy to purify IL-27 and to separate EBI3 (SEQ ID NO: 6) from IL-27α^{L162C} (SEQ ID NO: 9), a stabilized IL-27α mutant⁴³ (see FIG. 3a and FIG. 8b). In both cases, the strategy of the inventors of the present invention allowed them to produce milligram-quantities of glycosylated pure protein from human cells. IL-12α^{C96S} (SEQ ID NO: 7) produced in this manner remained assembly-competent with IL-12β (SEQ ID NO: 4), and EBI3 (SEQ ID NO: 6) remained assembly-competent with IL-27α (see FIG. 8c). Furthermore, re-assembled IL-12 and IL-27 were biologically active and could induce receptor dimerization in a NanoBRET assay that the inventors of the present invention established for this purpose (see FIG. 3b) as well as signaling in NK-92 or BL-2 cells, respectively (see FIG. 3c). Together, the protocol of the inventors of the present invention yielded properly structured proteins. Far-UV CD spectroscopy confirmed this notion and revealed an alpha-helical structure for IL-12α^{C96S} (SEQ ID NO: 7) as expected from the crystal structure of the IL-12 molecule²⁰. EBI3 (SEQ ID NO: 6) showed the far-UV CD spectroscopic signature of a beta-sheet protein, containing some flexible regions, in agreement with available IL-27:receptor structures²⁶⁻²⁸ (see FIG. 3d). Apparent melting temperatures were 47±0.2° C. (IL-12α^{C96S}, SEQ ID NO: 7) and 50±0.2° C., respectively (EBI3, SEQ ID NO: 6) (see FIG. 3e). Thus, IL-12α^{C96S} (SEQ ID NO: 7) and EBI3 (SEQ ID NO: 6) are both relatively stable proteins, which analytical ultracentrifugation revealed to be mostly monomeric with some self-assembly observed for EBI3 (SEQ ID NO: 6) (see FIG. 9a). Surprisingly, although IL-12α^{C96S} (SEQ ID NO: 7) and EBI3 (SEQ ID NO: 6) were correctly structured, both proteins appeared not to assemble with each other to a detectable amount in vitro as judged by analytical ultracentrifugation, co-immunoprecipitation, and hydrogen-deuterium exchange mass spectrometry experiments (see FIG. 9a-9c). Taken together, these detailed biophysical and functional analyses revealed that IL-12α^{C69S} (SEQ ID NO: 7) and EBI3 (SEQ ID NO: 6) can be purified as stable, well-folded proteins, but that IL-35, in contrast to IL-12 and IL-27, cannot be readily formed from its subunits in vitro. This adds even more importance to the findings of the inventors of the present invention that IL-12α (SEQ ID NO: 3) and EBI3 (SEQ ID NO: 6) can be secreted individually and puts these as independent cytokine-related molecules on the agenda. Thus, the inventors of the present invention continued with functional studies on IL-12α (SEQ ID NO: 3) and EBI3 (SEQ ID NO: 6).

### Example 3: IL-12α and EBI3 have Anti-Inflammatory Properties

IL-35 is described as an immunosuppressive cytokine that affects signaling in various cell types⁶. Since the data of the inventors of the present invention showed secretion of free IL-12α (SEQ ID NO: 3) and EBI3 (SEQ ID NO: 6) from cells producing both IL-35 subunits, the inventors of the present invention wondered if immunological effects could also be exerted by these subunits as recent studies indicated³⁰⁻³³. Pure human IL-12α (SEQ ID NO: 3) and EBI3 (SEQ ID NO: 6) produced in mammalian cells have not yet been investigated in this regard, but were now available to the inventors of the present invention. To assess functions of IL-12α^{C96S} (SEQ ID NO: 7) and EBI3 (SEQ ID NO: 6), the inventors of the present invention used primary human peripheral blood mononuclear cells (PBMCs) to study effects on cells of the innate and adaptive immune system. Since IL-35 acts as an immunosuppressor, the inventors of the present invention focused on anti-inflammatory effects of IL-12α^{C69S} (SEQ ID NO: 7) or EBI3 (SEQ ID NO: 6) as IL-35 subunits. Thus, the inventors of the present invention treated PBMCs with LPS to induce inflammatory responses and tested for effects of both, IL-12α^{C96S} (SEQ ID NO: 7) and EBI3 (SEQ ID NO: 6). In agreement with a globally anti-inflammatory role, the transcription of the pro-inflammatory cytokines IL1B, IL6, CXCL8 and TNFA was reduced by both IL-12α^{C96S} (SEQ ID NO: 7) and EBI3 (SEQ ID NO: 6), whereas the levels of the immunosuppressive cytokine TGFB remained unaffected (see FIG. 10). In general, in agreement with these transcriptional effects, also the secretion of these pro-inflammatory cytokines was significantly downregulated by EBI3 (SEQ ID NO: 6), while effects of IL-12α^{C69S} (SEQ ID NO: 7) were less pronounced (see FIG. 4a).

As the inventors of the present invention found pro-inflammatory cytokines of the monocyte/macrophage compartment to be affected by IL-12α^{C96S} (SEQ ID NO: 7) and EBI3 (SEQ ID NO: 6), the inventors of the present invention decided to further investigate if these cells were affected by both proteins. Lung macrophages are essential players in the development of allergic airway diseases such as asthma, where IL-35 (and thus possibly IL-12α and/or EBI3) play important roles⁴⁶. Therefore, to assess whether IL-12α^{C69S} (SEQ ID NO: 7) and EBI3 (SEQ ID NO: 6) can modulate pro-inflammatory cytokine production by myeloid cells, the inventors of the present invention isolated CD14⁺ monocytes from PBMCs, differentiated them into alveolar-like macrophages by stimulation with GM-CSF and TGF-β^{47,48} (see FIG. 4b), and treated them with house dust mite extract (HDM), one of the most frequent triggers of allergic asthma. Treatment of the cells with either IL-12α^{C96S} (SEQ ID NO: 7) or EBI3 (SEQ ID NO: 6) after HDM stimulation revealed a significant suppression of the pro-inflammatory cytokines, IL-1β, IL-6, IL-8, and TNFα (see FIG. 4c). Thus, IL-12α^{C69S} (SEQ ID NO: 7) and EBI3 (SEQ ID NO: 6) might act as immunosuppressors in settings of e.g. bacterial or allergenic challenge.

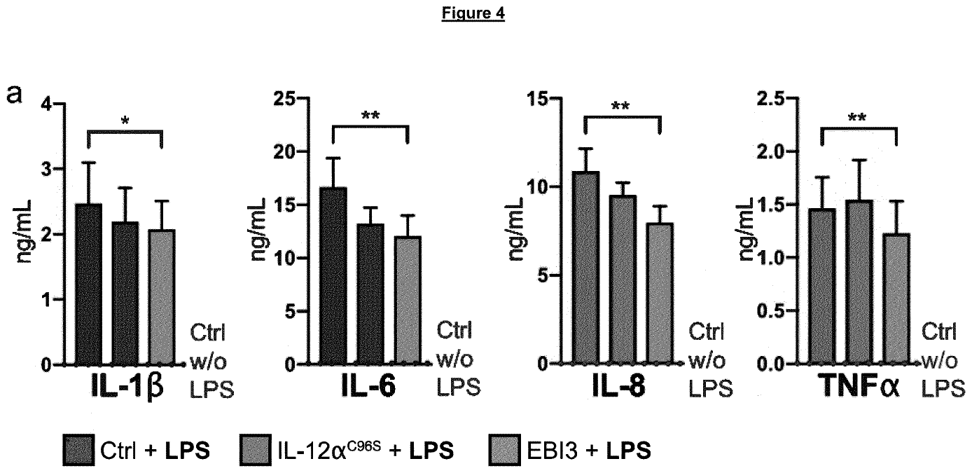

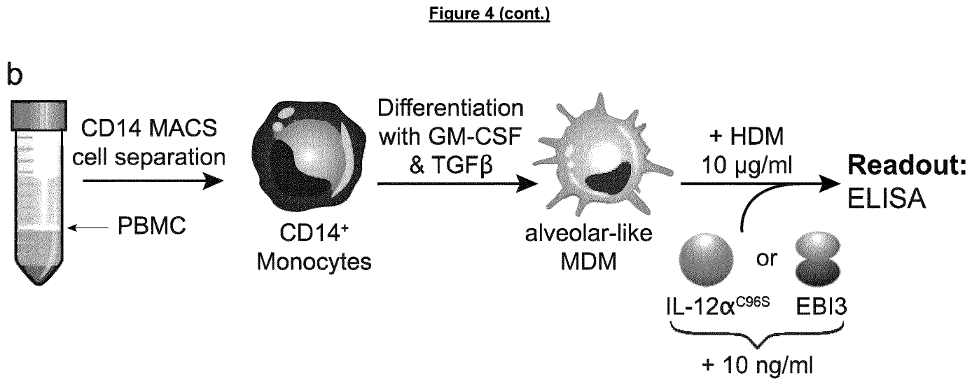

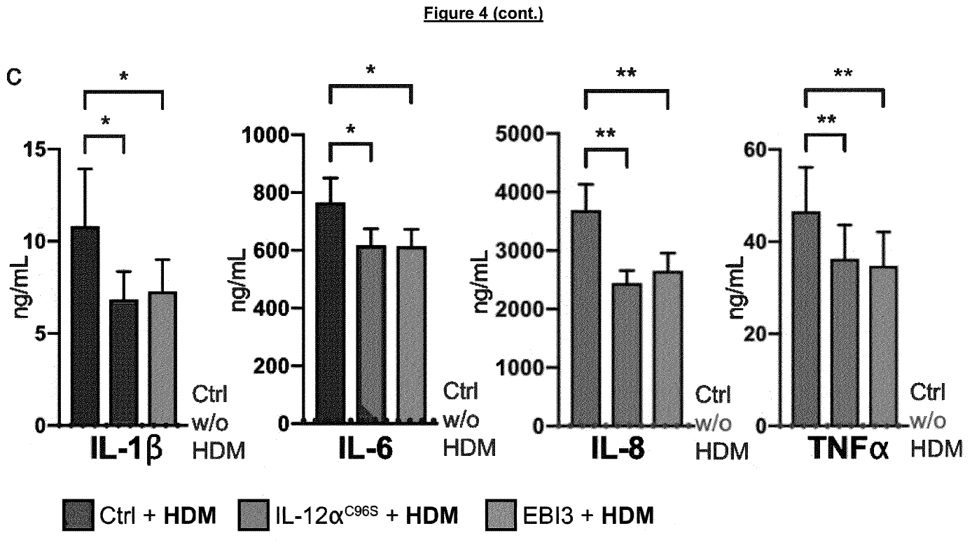

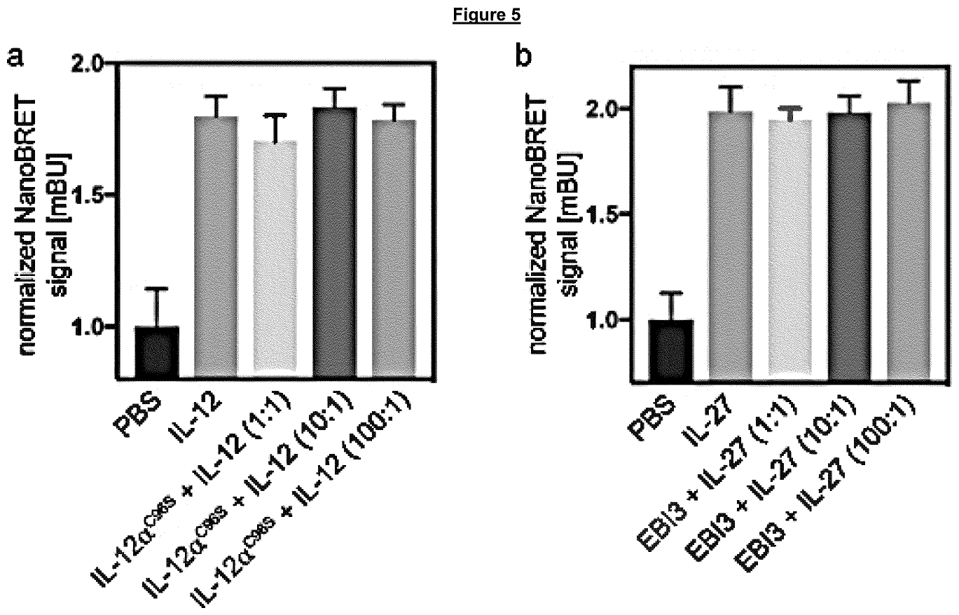

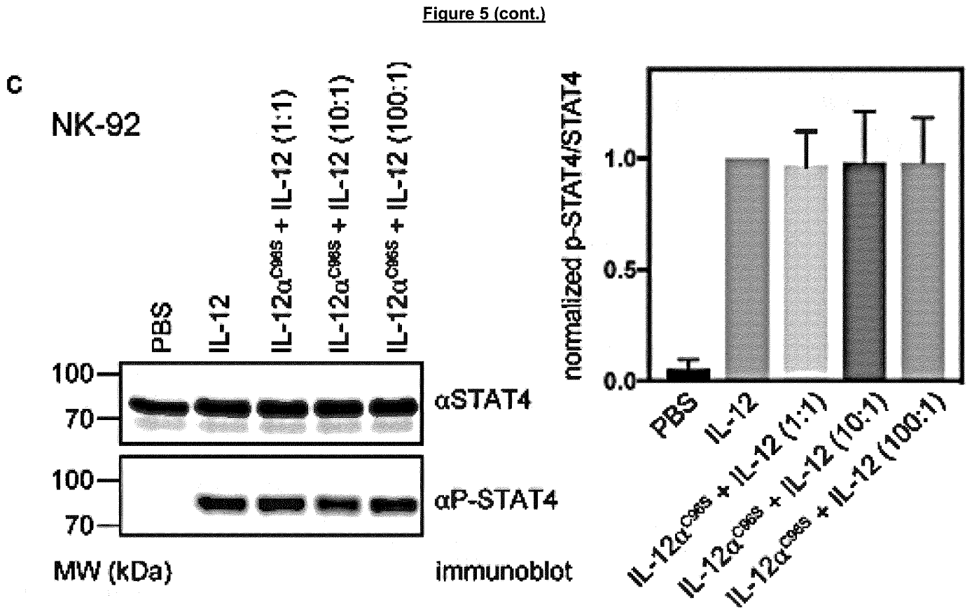

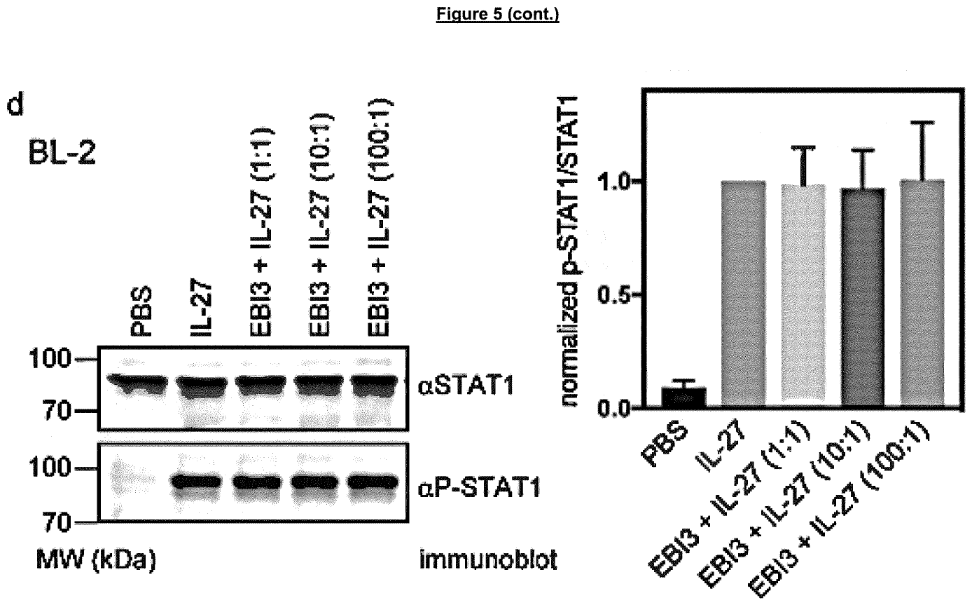

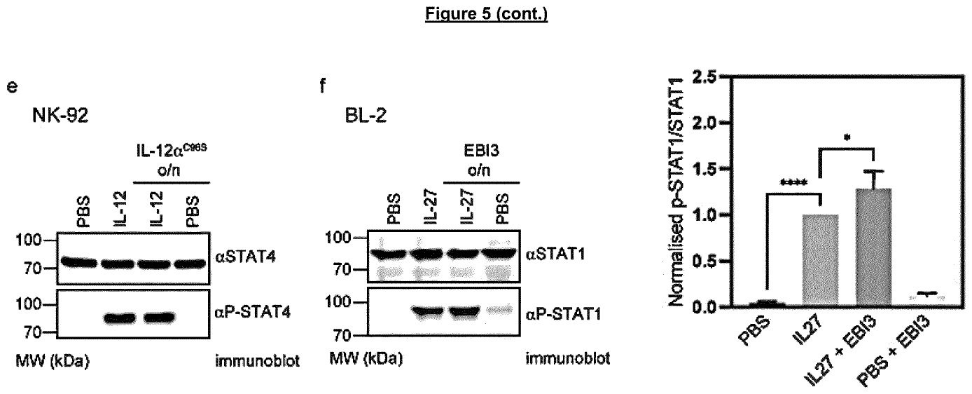

In the presence of pro-inflammatory stimuli, PBMCs and macrophages secrete IL-12 (SEQ ID NOs: 3 and 4) and IL-27 (SEQ ID NO: 5 and 6)^{49,50}. Thus, it is possible that IL-12α^{C69S} (SEQ ID NO: 7) inhibits IL-12-mediated pro-inflammatory effects and that EBI3 (SEQ ID NO: 6) does the same for IL-27 signaling. This would explain the anti-inflammatory effects the inventors of the present invention observed for both subunits. To test for this possibility, the inventors of the present invention applied the NanoBRET reporter system. In this experiment, a 30-min pre-incubation with IL-12α^{C69S} (SEQ ID NO: 7) did not inhibit IL-12-induced receptor dimerization, even at a 100-fold excess over the heterodimeric cytokine (see FIG. 5a). Analogously, pre-incubation with EBI3 (SEQ ID NO: 6) did not inhibit IL-27-induced receptor dimerization (see FIG. 5b). When the inventors of the present invention used IL-12-responsive NK-92 cells as a further test, also no significant reduction in IL-12-induced STAT4 phosphorylation was observed in the presence of an excess of IL-12α^{C96S} (SEQ ID NO: 7) over IL-12 (SEQ ID NOs: 3 and 4) (see FIG. 5c). Similarly, pre-incubation of IL-27 responsive BL-2 cells with EBI3 (SEQ ID NO: 6) did not lead to reduced IL-27 signaling (see FIG. 5d).

A further possibility how IL-12α^{C96S} (SEQ ID NO: 7) and EBI3 (SEQ ID NO: 6) could reduce signaling of the heterodimeric cytokines is by influencing receptor chain internalization, as has been described for monomeric IL-12β (SEQ ID NO: 4)⁵¹. To test for this possibility, the inventors of the present invention pre-treated NK-92 or BL-2 cells with IL-12α^{C96S} (SEQ ID NO: 7) or EBI3 (SEQ ID NO: 6) overnight, respectively. After this pre-treatment, the respective heterodimeric cytokines were added (IL-12 or IL-27) to NK-92 or BL-2 cells, with a further co-addition of IL-12α^{C96S} (SEQ ID NO: 7) or EBI3 (SEQ ID NO: 6), respectively. Like in the short-term treatment experiments (see FIGS. 5c and 5d), no reduced STAT phosphorylation was observed in either condition (see FIGS. 5e and 5f). In contrast, overnight incubation with EBI3 (SEQ ID NO: 6) itself led to a weak STAT1 phosphorylation signal in BL2 cells that was additive with the IL-27-induced signal (see FIG. 5f).

Together, the data of the inventors of the present invention indicate that IL-12α^{C96S} (SEQ ID NO: 7) and EBI3 (SEQ ID NO: 6) do not inhibit signaling of their related heterodimeric cytokines, but that they rather exert their anti-inflammatory effects as active signaling molecules, which led us to further study their effects on immune cell differentiation.

### Example 4: IL-12α and EBI3 Act as Immunosuppressors by Inducing Regulatory T Cells and Reducing IL-4 Production by Th2 Cells

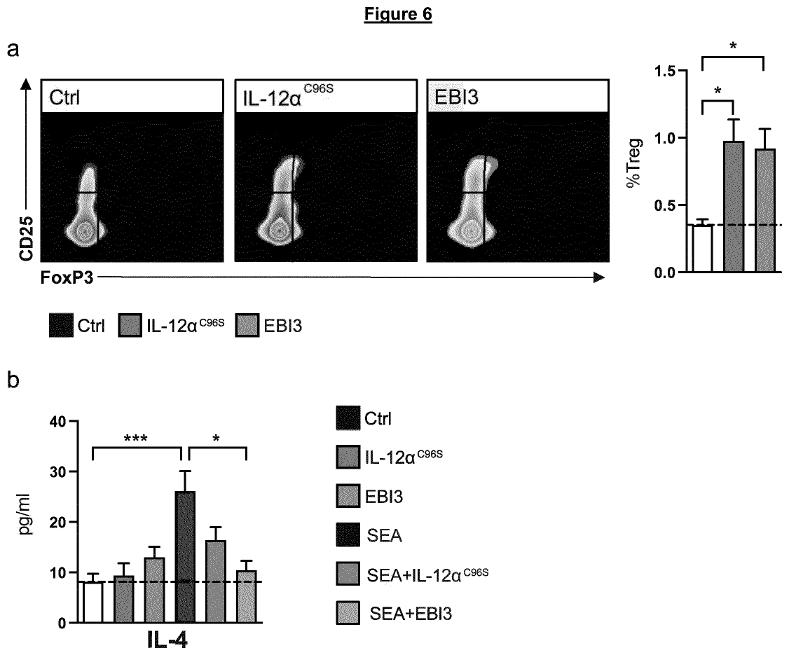

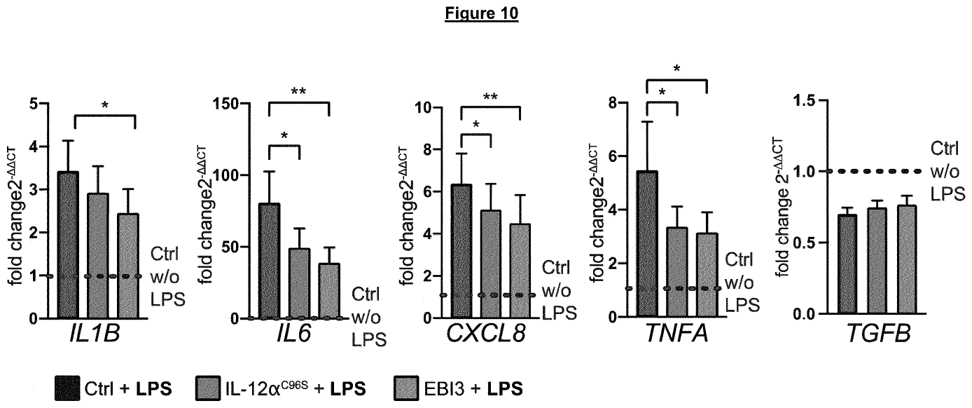

IL-35 was initially found to cause primary immunosuppression of effector T cell responses⁸ and its inhibitory role is further mediated by the induction of Treg cells⁵². To investigate if IL-12α (SEQ ID NO: 3) and EBI3 (SEQ ID NO: 6) can exert the same effects and thus suggest Treg cells as one mediator of their suppressive properties, the inventors of the present invention assessed the potential of both subunits to potentiate these cells in human PBMC cultures. Indeed, the inventors of the present invention were able to observe an induction/expansion of CD127-CD25⁺FoxP3⁺ regulatory T cells after stimulation with both IL-12α^{C96S} (SEQ ID NO: 7) and EBI3 (SEQ ID NO: 6), with again a more pronounced effect for EBI3 independent of TCR-stimulation or the addition of additional cytokines, such as IL-2 or TGFβ (see FIG. 6a). This observation confirms that IL-12α (SEQ ID NO: 3) and EBI3 (SEQ ID NO: 6) mimic major immunosuppressive functions attributed to the heterodimeric IL-35 (SEQ ID NOs: 1 and 2). Since the data of the inventors of the present invention pointed towards immunological effects mediated by both IL-12α (SEQ ID NO: 3) and EBI3 (SEQ ID NO: 6), the inventors of the present invention aimed to further examine IL-12α (SEQ ID NO: 3) and EBI3 (SEQ ID NO: 6) in disease-relevant settings using in vitro approaches. As it has been reported that IL-35 can suppress type 2 cytokine production (e.g. IL-4, IL-5) and show a beneficial impact on allergic disease⁵³, the inventors of the present invention analyzed the capacity of its subunits to reduce type 2 cytokine production following stimulation with LPS and Schistosoma mansoni soluble egg antigen (SEA), a strong parasitic inflammatory trigger known to induce Th2 immune responses. In this setting, the inventors of the present invention observed a significant suppression of IL-5, even at low gene expression levels, by both IL-12α^{C96S} (SEQ ID NO: 7) or EBI3 (SEQ ID NO: 6) in combination with LPS stimulation (see FIG. 11). To further confirm this result, the inventors of the present invention incubated PBMCs with SEA and simultaneously with either IL-12α^{C69S} (SEQ ID NO: 7) or EBI3 (SEQ ID NO: 6). Whereas IL-12α^{C69S} (SEQ ID NO: 7) only revealed a mild suppression of SEA-induced IL-4 production, EBI3 (SEQ ID NO. 6) strongly reduced IL-4 secretion after SEA treatment (see FIG. 6b). These data suggest that both IL-35 subunits (SEQ ID NOs: 1 and 2) also show clinically relevant immunosuppressive capacities in type-2 inflammatory settings.

### Summary

IL-35 is the structurally least-well defined member of the IL-12 family and its engagement of multiple receptors has remained enigmatic. The study of the inventors of the present invention now shows that cells expressing both IL-35 subunits, IL-12α (SEQ ID NO: 3) and EBI3 (SEQ ID NO: 6), do not only secrete heterodimeric IL-35, but also both of its non-assembled subunits, which is in contrast to other heterodimeric human IL-12 family members³. In the setting of the inventors of the present invention, secretion of the non-heterodimeric subunits significantly exceeds secretion of IL-35, a finding, which will be relevant to further tests in primary immune cells. The data of the inventors of the present invention revealed IL-35 subunits (SEQ ID NOs: 1 and 2) to be stable proteins in isolation that exert anti-inflammatory effects on primary human PBMCs and macrophages under a type-1 or different type-2 inflammatory stimuli. HDM represents a major source of indoor allergens and is strongly associated with the development of asthma. Macrophages, as part of the innate immune system, are among the first and most abundant cells in the lung to encounter allergens and contribute to chronic airway inflammation like asthma through the production of pro-inflammatory cytokines^{54,55}. The findings of the inventors of the present invention suggest that IL-12α (SEQ ID NO: 3) and EBI3 (SEQ ID NO: 6) may account for some of the effects in asthma that so far have been ascribed to IL-35^{9,46,56} and may thus be relevant to study further. One of these findings is the ability to induce regulatory T cells⁸, which aligns well with the capacities of both EBI3 (SEQ ID NO: 6) and IL-12α^{C69S} (SEQ ID NO: 7) to suppress type 2 cytokines. In addition to allergens, parasitic infections trigger strong type 2 immune responses. The inventors of the present invention were able to show suppressive capacities of EBI3 (SEQ ID NO: 6) and IL-12α^{C69S} (SEQ ID NO: 7) on both allergen- and parasite-induced type 2 inflammatory responses with EBI3 (SEQ ID NO: 6) exhibiting more pronounced effects. Although the effects were mild, they were even observed in a complex cell pool with only low IL-12α/EBI3 (SEQ ID NO: 3/SEQ ID NO: 6) concentrations. The generally immunosuppressive functions of IL-12α^{C96S} (SEQ ID NO: 7) and EBI3 (SEQ ID NO: 6) are in agreement with previous studies^{30,31,57,58}. Importantly, the inventors of the present invention extend these immunological data by demonstrating that these molecules are actually secreted from cells expressing both subunits and thus underline their relevance in vivo. The inventors of the present invention furthermore do not find evidence that IL-12α^{C96S} (SEQ ID NO: 7) inhibits IL-12 signaling or that EBI3 (SEQ ID NO: 6) inhibits IL-27 signaling, suggesting that IL-12α (SEQ ID NO: 3) and EBI3 (SEQ ID NO: 6) may be considered proper, active cytokines in the human immune system. These findings have several major implications for the understanding of the human IL-12 family and their role in regulating immune responses.

The independent functions of IL-12α (SEQ ID NO: 3) and EBI3 (SEQ ID NO: 6) suggest that some of the effects attributed to IL-35 may in fact be exerted by its subunits, also since functional studies on IL-12 family cytokines are often performed in subunit-knockout animals so that potentially more than one factor is lost. Instability and/or low assembly propensity of IL-35 might balance effects that are either mediated by the heterodimer or the individual subunits. Recent data for IL-6 signaling consider sIL-6R and sgp130 as a systemic buffer for their ability to transiently bind IL-6, which might lead to an increased half-life and enables classical- and trans-signaling^{59,60}.

Recent studies have shown that IL-12 can be reconstituted from IL-12α (SEQ ID NO: 3) and IL-12β (SEQ ID NO: 4) derived from different cells⁶², but the mechanisms of IL-12α (SEQ ID NO: 3) secretion, which normally is retained in cells in isolation⁴⁰, had remained unclear. The study of the inventors of the present invention shows how cells can secrete IL-12α (SEQ ID NO: 3) and add an important further functional layer: IL-12α (SEQ ID NO: 3) alone is anti-inflammatory as the data of the inventors of the present invention show, yet it can pair with IL-12β (SEQ ID NO: 4) to form pro-inflammatory IL-12. The absence or presence of IL-12β (SEQ ID NO: 4) expressing cells may thus have a pronounced effect on the immune functions of adjacent cells expressing other IL-12 family subunits. In the light of this finding, it is also noteworthy that IL-12 producing cells secrete an excess of free IL-12β (SEQ ID NO: 4) that may inhibit IL-12 signaling, act as a signaling molecule by itself or, as the data of the inventors of the present invention show, pair with IL-12β (SEQ ID NO: 4) to further increase extracellular IL-12 levels. Analogously, EBI3 (SEQ ID NO: 6) may have distinct effects in acting alone or pairing with other cytokines outside the cell^{57,62}. Thus, the combinatorial complexity of IL-12 cytokine family signaling is even larger than previously thought and may be highly context-dependent.

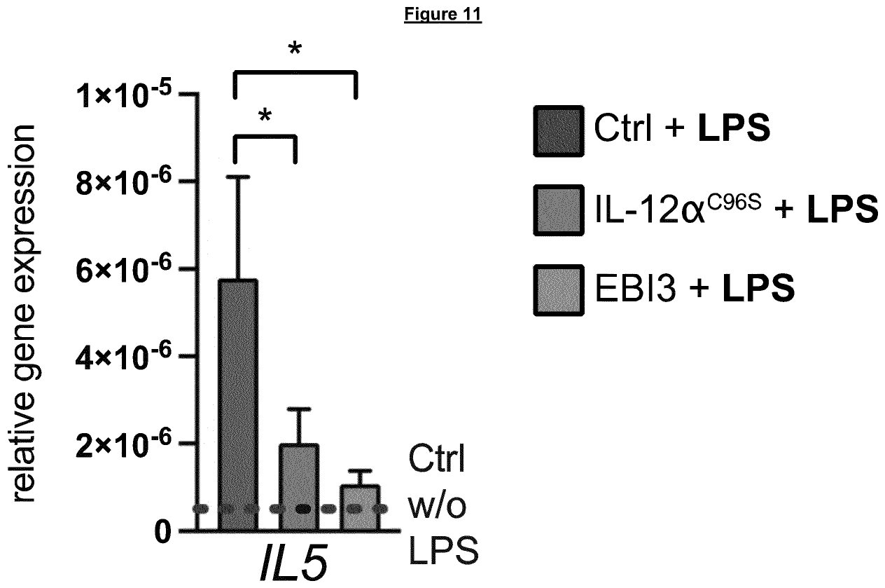

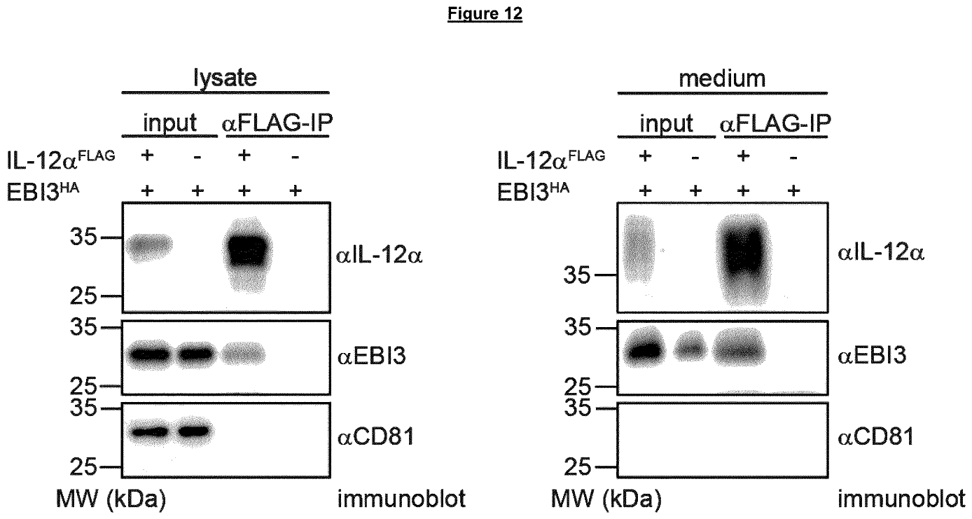

One finding from the study of the inventors of the present invention is that cells expressing IL-12α (SEQ ID NO: 3) and EBI3 (SEQ ID NO: 6) secrete assembled IL-35, but that IL-12α^{C69S} (SEQ ID NO: 7) and EBI3 (SEQ ID NO: 6) could apparently not reconstitute IL-35 in vitro, although the subunits remained assembly-competent to form other cytokines (IL-12 and IL-27) as the data of the inventors of the present invention show. Alternatively, complexes formed in vitro may be too unstable or of too low abundance to be detected properly. Recent work suggested CD81 to potentially assemble with IL-35⁶³. The inventors of the present invention could not detect CD81 in complex with IL-12α (SEQ ID NO: 3) and EBI3 (SEQ ID NO: 6), arguing against it being an essential component of IL-35 (see FIG. 12).

In summary, the study of the inventors of the present invention reveals that IL-35, in contrast to other IL-12 family members, is not secreted as a strict heterodimer, but assembly-induced folding and transient interactions enable the secretion of its subunits. The discovery of the inventors of the present invention that IL-35 is in fact a compound of both assembled and unassembled IL-12α (SEQ ID NO: 3) and EBI3 (SEQ ID NO: 6) might provide a molecular explanation for the pleiotropic signaling pathways and effects that are observed for IL-35. This is further supported by autonomous immunosuppressive properties of both subunits in primary human immune cells and their capabilities to induce regulatory T cells. Due to its potent immunosuppressive effects in the tumor microenvironment—but also its low levels in autoimmune diseases, IL-35 is a highly attractive therapeutic target or drug candidate. While immunological effects should be re-evaluated in further cell models, the data of the inventors of the present invention suggest that IL-12α (SEQ ID NO: 3) and EBI3 (SEQ ID NO: 6) should be included in this consideration, as they are secreted from cells and have anti-inflammatory effects.

### The Invention is Further Characterized by the Following Items:

### Items:

1. A method for producing the α-subunit of human Interleukin 35 (SEQ ID NO: 1) from human Interleukin 12 and/or the β-subunit EBI3 of human Interleukin 35 (SEQ ID NO: 2) from human Interleukin 27, comprising the steps of:
(a) introducing into a nucleic acid molecule encoding the recombinant α- and β-subunits of the human Interleukin 12 polypeptide (SEQ ID NOs: 3 and 4) a nucleotide sequence mutating at least one amino acid residue of the α-subunit of human Interleukin 12 (SEQ ID NO: 3) and/or mutating at least one amino acid residue of the β-subunit of human Interleukin 12 (SEQ ID NO: 4), and/or
(b) introducing into a nucleic acid molecule encoding the recombinant α- and β-subunits of the human Interleukin 27 polypeptide (SEQ ID NOs: 5 and 6) a nucleotide sequence mutating at least one amino acid residue of the α-subunit of human Interleukin 27 (SEQ ID NO: 5), and
(c) introducing the obtained nucleic acid molecule(s) of steps (a) and/or (b) for expression into a suitable host cell or into a suitable cell extract or cell lysate.
2. The method according to item 1, wherein the method further comprises a step (d) comprising the isolation of human Interleukin 12 and/or the isolation of human Interleukin 27 from the product of step (c).
3. The method according to item 2, wherein the isolation of human Interleukin 12 and/or the isolation of human Interleukin 27 is/are carried out by affinity chromatography.
4. The method according to item 2 or item 3, wherein the method further comprises a step (e) comprising the separation of the α-subunit of human Interleukin 12 (SEQ ID NO: 3) from the isolated human Interleukin 12 of step (d) and/or the separation of the β-subunit of human Interleukin 27 (SEQ ID NO: 6) from the isolated human Interleukin 27 of step (d).
5. The method according to item 4, wherein the separation of the α-subunit of human Interleukin 12 (SEQ ID NO: 3) from the isolated human Interleukin 12 of step (d) and/or the separation of the β-subunit of human Interleukin 27 (SEQ ID NO: 6) from the isolated human Interleukin 27 of step (d) is carried out by denaturation and subsequent renaturation under physiological conditions.
6. The method according to item 4 or item 5, wherein the method further comprises a step (f) comprising the purification of the α-subunit of human Interleukin 12 (SEQ ID NO: 3) of step (e) and/or the purification of the β-subunit of human Interleukin 27 (SEQ ID NO: 6) of step (e).
7. The method according to item 6, wherein the purification of the α-subunit of human Interleukin 12 (SEQ ID NO: 3) of step (e) and/or the purification of the β-subunit of human Interleukin 27 (SEQ ID NO: 6) of step (e) is/are carried out by size exclusion chromatography.
8. The method according to any one of the preceding items 1 to 7, wherein the method comprises the steps of:
(a) introducing into a nucleic acid molecule encoding the recombinant α- and β-subunits of the human Interleukin 12 polypeptide (SEQ ID NOs: 3 and 4) a nucleotide sequence mutating one amino acid residue of the α-subunit of human Interleukin 12 (SEQ ID NO: 3) and/or mutating one amino acid residue of the β-subunit of human Interleukin 12 (SEQ ID NO: 4), and/or
(b) introducing into a nucleic acid molecule encoding the recombinant α- and β-subunits of the human Interleukin 27 polypeptide (SEQ ID NOs: 5 and 6) a nucleotide sequence mutating one amino acid residue of the α-subunit of human Interleukin 27 (SEQ ID NO: 5), and
(c) introducing the obtained nucleic acid molecule(s) of steps (a) and/or (b) for expression into a suitable host cell or into a suitable cell extract or cell lysate.
9. The method according to any one of the preceding items 1 to 7, wherein the method comprises the steps of:
(a) introducing into a nucleic acid molecule encoding the recombinant α- and β-subunits of the human Interleukin 12 polypeptide (SEQ ID NOs: 3 and 4) a nucleotide sequence mutating one amino acid residue of the α-subunit of human Interleukin 12 (SEQ ID NO: 3), and/or
(b) introducing into a nucleic acid molecule encoding the recombinant α- and β-subunits of the human Interleukin 27 polypeptide (SEQ ID NO: 5 and 6) a nucleotide sequence mutating one amino acid residue of the α-subunit of human Interleukin 27 (SEQ ID NO: 5), and
(c) introducing the obtained nucleic acid molecule(s) of steps (a) and/or (b) for expression into a suitable host cell or into a suitable cell extract or cell lysate.
10. The method according to any one of the preceding items 1 to 7, wherein the method comprises the steps of:
(a) introducing into a nucleic acid molecule encoding the recombinant α- and β-subunits of the human Interleukin 12 polypeptide (SEQ ID NOs: 3 and 4) a nucleotide sequence mutating one amino acid residue of the β-subunit of human Interleukin 12 (SEQ ID NO: 4), and/or
(b) introducing into a nucleic acid molecule encoding the recombinant α- and β-subunits of the human Interleukin 27 polypeptide (SEQ ID NO: 5 and 6) a nucleotide sequence mutating one amino acid residue of the α-subunit of human Interleukin 27 (SEQ ID NO: 5), and
(c) introducing the obtained nucleic acid molecule(s) of steps (a) and/or (b) for expression into a suitable host cell or into a suitable cell extract or cell lysate.
11. The method according to any one of the preceding items 1 to 7, wherein the method comprises the steps of:
(a) introducing into a nucleic acid molecule encoding the recombinant α- and β-subunits of the human Interleukin 12 polypeptide (SEQ ID NOs: 3 and 4) a nucleotide sequence mutating at least one amino acid residue of the α-subunit of human Interleukin 12 (SEQ ID NO: 3), and
(b) introducing the obtained nucleic acid molecule of step (a) for expression into a suitable host cell or into a suitable cell extract or cell lysate.
12. The method according to any one of the preceding items 1 to 7, wherein the method comprises the steps of:
(a) introducing into a nucleic acid molecule encoding the recombinant α- and β-subunits of the human Interleukin 12 polypeptide (SEQ ID NOs: 3 and 4) a nucleotide sequence mutating one amino acid residue of the α-subunit of human Interleukin 12 (SEQ ID NO: 3), and
(b) introducing the obtained nucleic acid molecule of step (a) for expression into a suitable host cell or into a suitable cell extract or cell lysate.
13. The method according to any one of the preceding items 1 to 7, wherein the method comprises the steps of:
(a) introducing into a nucleic acid molecule encoding the recombinant α- and β-subunits of the human Interleukin 12 polypeptide (SEQ ID NOs: 3 and 4) a nucleotide sequence mutating at least one amino acid residue of the β-subunit of human Interleukin 12 (SEQ ID NO: 4), and
(b) introducing the obtained nucleic acid molecule of step (a) for expression into a suitable host cell or into a suitable cell extract or cell lysate.
14. The method according to any one of the preceding items 1 to 7, wherein the method comprises the steps of:
(a) introducing into a nucleic acid molecule encoding the recombinant α- and β-subunits of the human Interleukin 12 polypeptide (SEQ ID NOs: 3 and 4) a nucleotide sequence mutating one amino acid residue of the β-subunit of human Interleukin 12 (SEQ ID NO: 4), and
(b) introducing the obtained nucleic acid molecule of step (a) for expression into a suitable host cell or into a suitable cell extract or cell lysate.
15. The method according to any one of the preceding items 1 to 7, wherein the method comprises the steps of:
(a) introducing into a nucleic acid molecule encoding the recombinant α- and β-subunits of the human Interleukin 27 polypeptide (SEQ ID NOs: 5 and 6) a nucleotide sequence mutating at least one amino acid residue of the α-subunit of human Interleukin 27 (SEQ ID NO: 5), and
(b) introducing the obtained nucleic acid molecule of step (a) for expression into a suitable host cell or into a suitable cell extract or cell lysate.
16. The method according to any one of the preceding items 1 to 7, wherein the method comprises the steps of:
(a) introducing into a nucleic acid molecule encoding the recombinant α- and β-subunits of the human Interleukin 27 polypeptide (SEQ ID NOs: 5 and 6) a nucleotide sequence mutating one amino acid residue of the α-subunit of human Interleukin 27 (SEQ ID NO: 5), and
(b) introducing the obtained nucleic acid molecule of step (a) for expression into a suitable host cell or into a suitable cell extract or cell lysate.
17. The method according to any one of the preceding items 1 to 7, wherein the method comprises the steps of:
(a) introducing into a nucleic acid molecule encoding the recombinant α- and β-subunits of the human Interleukin 12 polypeptide (SEQ ID NOs: 3 and 4) a nucleotide sequence mutating at least one, preferably one, amino acid residue of the α-subunit of human Interleukin 12 (SEQ ID NO: 3) and/or mutating at least one, preferably one, amino acid residue of the β-subunit of human Interleukin 12 (SEQ ID NO: 4), and/or
(b) introducing into a nucleic acid molecule encoding the recombinant α- and β-subunits of the human Interleukin 27 polypeptide (SEQ ID NOs: 5 and 6) the nucleotide sequence encoding the wild type of the α-subunit of human Interleukin 27 (SEQ ID NO: 5), and
(c) introducing the obtained nucleic acid molecule(s) of steps (a) and/or (b) for expression into a suitable host cell or into a suitable cell extract or cell lysate.
18. The method according to any one of the preceding items 1 to 7, wherein the method comprises the steps of:
(a) introducing into a nucleic acid molecule encoding the recombinant α- and β-subunits of the human Interleukin 12 polypeptide (SEQ ID NOs: 3 and 4) a nucleotide sequence encoding the wild type of the α-subunit of human Interleukin 12 (SEQ ID NO: 3) and a nucleotide sequence mutating at least one, preferably one, amino acid residue of the β-subunit of human Interleukin 12 (SEQ ID NO: 4), and/or
(b) introducing into a nucleic acid molecule encoding the recombinant α- and β-subunits of the human Interleukin 27 polypeptide (SEQ ID NOs: 5 and 6) a nucleotide sequence mutating at least one, preferably one, amino acid residue of the α-subunit of human Interleukin 27 (SEQ ID NO: 5), and
(c) introducing the obtained nucleic acid molecule(s) of steps (a) and/or (b) for expression into a suitable host cell or into a suitable cell extract or cell lysate.
19. The method according to any one of the preceding items 1 to 7, wherein the method comprises the steps of:
(a) introducing into a nucleic acid molecule encoding the recombinant α- and β-subunits of the human Interleukin 12 polypeptide (SEQ ID NOs: 3 and 4) a nucleotide sequence mutating at least one, preferably one, amino acid residue of the α-subunit of human Interleukin 12 (SEQ ID NO: 3) and a nucleotide sequence encoding the wild type of the β-subunit of human Interleukin 12 (SEQ ID NO: 4), and/or
(b) introducing into a nucleic acid molecule encoding the recombinant α- and β-subunits of the human Interleukin 27 polypeptide (SEQ ID NOs: 5 and 6) a nucleotide sequence mutating at least one, preferably one, amino acid residue of the α-subunit of human Interleukin 27 (SEQ ID NO: 5), and
(c) introducing the obtained nucleic acid molecule(s) of steps (a) and/or (b) for expression into a suitable host cell or into a suitable cell extract or cell lysate.
20. The method according to any one of the preceding items 1 to 7, wherein the method comprises the steps of:
(a) introducing into a nucleic acid molecule encoding the recombinant α- and β-subunits of the human Interleukin 12 polypeptide (SEQ ID NOs: 3 and 4) a nucleotide sequence mutating at least one, preferably one, amino acid residue of the α-subunit of human Interleukin 12 (SEQ ID NO: 3) and mutating at least one, preferably one, amino acid residue of the β-subunit of human Interleukin 12 (SEQ ID NO: 4), and
(b) introducing the obtained nucleic acid molecule of step (a) for expression into a suitable host cell or into a suitable cell extract or cell lysate.
21. The method according to any one of the preceding items 1 to 7, wherein the method comprises the steps of:
(a) introducing into a nucleic acid molecule encoding the recombinant α- and β-subunits of the human Interleukin 12 polypeptide (SEQ ID NOs: 3 and 4) a nucleotide sequence encoding the wild type of the α-subunit of human Interleukin 12 (SEQ ID NO: 3) and a nucleotide sequence mutating at least one, preferably one, amino acid residue of the β-subunit of human Interleukin 12 (SEQ ID NO: 4), and
(c) introducing the obtained nucleic acid molecule(s) of steps (a) and/or (b) for expression into a suitable host cell or into a suitable cell extract or cell lysate.
22. The method according to any one of the preceding items 1 to 7, wherein the method comprises the steps of:
(a) introducing into a nucleic acid molecule encoding the recombinant α- and β-subunits of the human Interleukin 12 polypeptide (SEQ ID NOs: 3 and 4) a nucleotide sequence mutating at least one, preferably one, amino acid residue of the α-subunit of human Interleukin 12 (SEQ ID NO: 3) and a nucleotide sequence encoding the wild type of the β-subunit of human Interleukin 12 (SEQ ID NO: 4), and
(b) introducing the obtained nucleic acid molecule of step (a) for expression into a suitable host cell or into a suitable cell extract or cell lysate.
23. The method according to any one of the preceding items 1 to 7, wherein the method comprises the steps of:
(a) introducing into a nucleic acid molecule encoding the recombinant α- and β-subunits of the human Interleukin 27 polypeptide (SEQ ID NOs: 5 and 6) a nucleotide sequence mutating at least one, preferably one, amino acid residue of the α-subunit of human Interleukin 27 (SEQ ID NO: 5), and
(b) introducing the obtained nucleic acid molecule of step (a) for expression into a suitable host cell or into a suitable cell extract or cell lysate.
24. The method according to any one of the preceding items, wherein step (a) comprises introducing into a nucleic acid molecule encoding the recombinant α- and β-subunits of the human Interleukin 12 polypeptide (SEQ ID NOs: 3 and 4) a nucleotide sequence mutating the amino acid residue of the α-subunit of human Interleukin 12 at sequence position 96.
25. The method according to any one of the preceding items, wherein step (a) comprises introducing into a nucleic acid molecule encoding the recombinant α- and β-subunits of the human Interleukin 12 polypeptide (SEQ ID NOs: 3 and 4) a nucleotide sequence mutating the amino acid residue of the α-subunit of human Interleukin 12 at sequence position 96 to another amino acid than cysteine, preferably to serine or alanine, more preferably to serine.
26. The method according to item 24 or item 25, wherein the nucleotide sequence further comprises a nucleotide sequence encoding an affinity tag.
27. The method according to any one of the preceding items, wherein step (a) comprises introducing into a nucleic acid molecule encoding the recombinant α- and β-subunits of the human Interleukin 12 polypeptide (SEQ ID NOs: 3 and 4) a nucleotide sequence mutating the amino acid residue of the β-subunit of human Interleukin 12 at sequence position 199.
28. The method according to any one of the preceding items, wherein step (a) comprises introducing into a nucleic acid molecule encoding the recombinant α- and β-subunits of the human Interleukin 12 polypeptide (SEQ ID NOs: 3 and 4) a nucleotide sequence mutating the amino acid residue of the β-subunit of human Interleukin 12 at sequence position 199 to another amino acid than cysteine, preferably to serine.
29. The method according to any one of the preceding items, wherein step (a) comprises introducing into a nucleic acid molecule encoding the recombinant α- and β-subunits of the human Interleukin 12 polypeptide (SEQ ID NOs: 3 and 4) a nucleotide sequence mutating the amino acid residue of the α-subunit of human Interleukin 12 at sequence position 96 and introducing into a nucleic acid molecule encoding the recombinant α- and β-subunits of the human Interleukin 12 polypeptide (SEQ ID NOs: 3 and 4) a nucleotide sequence mutating the amino acid residue of the β-subunit of human Interleukin 12 at sequence position 199.
30. The method according to any one of the preceding items, wherein step (a) comprises introducing into a nucleic acid molecule encoding the recombinant α- and β-subunits of the human Interleukin 12 polypeptide (SEQ ID NOs: 3 and 4) a nucleotide sequence mutating the amino acid residue of the α-subunit of human Interleukin 12 at sequence position 96 to serine and introducing into a nucleic acid molecule encoding the recombinant α- and β-subunits of the human Interleukin 12 polypeptide (SEQ ID NOs: 3 and 4) a nucleotide sequence mutating the amino acid residue of the β-subunit of human Interleukin 12 at sequence position 199 to serine.
31. The method according to any one of the preceding items, wherein step (b) comprises introducing into a nucleic acid molecule encoding the recombinant α- and β-subunits of the human Interleukin 27 polypeptide (SEQ ID NOs: 5 and 6) a nucleotide sequence mutating the amino acid residue of the α-subunit of human Interleukin 27 at sequence position 162.
32. The method according to any one of the preceding items, wherein step (b) comprises introducing into a nucleic acid molecule encoding the recombinant α- and β-subunits of the human Interleukin 27 polypeptide (SEQ ID NOs: 5 and 6) a nucleotide sequence mutating the amino acid residue of the α-subunit of human Interleukin 27 at sequence position 162 to cysteine.
33. The method according to item 31 or item 32, wherein the nucleotide sequence further comprises a nucleotide sequence encoding an affinity tag.
34. The method according to any one of the preceding items, wherein steps (a) and (b) are carried out simultaneously or step (a) is carried out before step (b) or step (b) is carried out before step (a).
35. The method according to any one of the preceding items, wherein step (a) comprises introducing into a nucleic acid molecule encoding the recombinant α- and β-subunits of the human Interleukin 12 polypeptide (SEQ ID NOs: 3 and 4) a nucleotide sequence mutating the amino acid residue of the α-subunit of human Interleukin 12 at sequence position 96, and wherein step (a) comprises introducing into a nucleic acid molecule encoding the recombinant α- and β-subunits of the human Interleukin 12 polypeptide (SEQ ID NOs: 3 and 4) a nucleotide sequence mutating the amino acid residue of the β-subunit of human Interleukin 12 at sequence position 199, and wherein step (b) comprises introducing into a nucleic acid molecule encoding the recombinant α- and β-subunits of the human Interleukin 27 polypeptide (SEQ ID NOs: 5 and 6) a nucleotide sequence mutating the amino acid residue of the α-subunit of human Interleukin 27 at sequence position 162.
36. The method according to any one of the preceding items, wherein step (a) comprises introducing into a nucleic acid molecule encoding the recombinant α- and β-subunits of the human Interleukin 12 polypeptide (SEQ ID NOs: 3 and 4) a nucleotide sequence mutating the amino acid residue of the α-subunit of human Interleukin 12 at sequence position 96 to serine, and wherein step (a) comprises introducing into a nucleic acid molecule encoding the recombinant α- and β-subunits of the human Interleukin 12 polypeptide (SEQ ID NOs: 3 and 4) a nucleotide sequence mutating the amino acid residue of the β-subunit of human Interleukin 12 at sequence position 199 to serine, and wherein step (b) comprises introducing into a nucleic acid molecule encoding the recombinant α- and β-subunits of the human Interleukin 27 polypeptide (SEQ ID NOs: 5 and 6) a nucleotide sequence mutating the amino acid residue of the α-subunit of human Interleukin 27 at sequence position 162 to cysteine.
37. The α-subunit of human Interleukin 12 (SEQ ID NO: 3) obtained according to any one of items 1 to 36.
38. The α-subunit of human Interleukin 12 (SEQ ID NO: 3) according to item 37, wherein said α-subunit of human Interleukin 12 is identical to the α-subunit of human Interleukin 35 (SEQ ID NO: 1).
39. The α-subunit of human Interleukin 12 (SEQ ID NO: 3) according to item 37 or 38, wherein the amino acid residue at amino acid position 96 is mutated.
40. The α-subunit of human Interleukin 12 (SEQ ID NO: 3) according to any one of items 37 to 39, wherein the amino acid residue at amino acid position 96 is mutated to serine.
41. The β-subunit of human Interleukin 27 (SEQ ID NO: 6) obtained according to the method of any one of items 1 to 36.
42. The β-subunit of human Interleukin 27 (SEQ ID NO: 6) according to item 41, wherein said β-subunit of human Interleukin 27 is identical to the β-subunit of human Interleukin 35 (SEQ ID NO: 2).
43. A nucleic acid molecule comprising a nucleotide sequence encoding the α-subunit of human Interleukin 12 (SEQ ID NO: 3) according to any one of items 37 to 40.
44. A nucleic acid molecule comprising a nucleotide sequence encoding the β-subunit of human Interleukin 27 (SEQ ID NO: 6) according to item 41 or item 42.
45. The nucleic acid molecule according to item 43 or item 44, wherein the nucleic acid molecule is operably linked to a regulatory sequence to allow expression of the nucleic acid molecule.
46. The nucleic acid molecule according to item 45, wherein the regulatory sequence comprises a promoter sequence.
47. The nucleic acid molecule of any one of items 43 to 46 comprised in a vector.
48. A host cell containing a nucleic acid molecule of any one of items 43 to 46.
49. An immune modulator comprising a subunit of any one of items 37 to 42.
50. Use of the α-subunit of human Interleukin 35 (SEQ ID NO: 1) and/or of the β-subunit of human Interleukin 35 (SEQ ID NO: 2), preferably of a subunit of any one of items 21 to 26, for the manufacture of a medicament for treating a disease in a mammal.
51. The use of item 50, wherein the mammal is a human.
52. The use of item 50 or item 51, wherein the disease is an infectious disease, an autoimmune disease, cancer, preferably breast cancer, pancreatic cancer or lung cancer, a transplantation-related disease, preferably Graft-versus-Host-disease, an inflammatory disease, preferably a chronic inflammatory disease or an acute inflammatory disease, more preferably a chronic inflammatory bowel disease, sepsis, septic shock, diabetes or an allergic disease, preferably asthma in a mammal.
53. The α-subunit of human Interleukin 35 (SEQ ID NO: 1) and/or the β-subunit of human Interleukin 35 (SEQ ID NO: 2), preferably the subunit of any one of items 37 to 42, for use as a medicament or a drug.
54. The α-subunit of human Interleukin 35 (SEQ ID NO: 1) and/or the β-subunit of human Interleukin 35 (SEQ ID NO: 2), preferably the subunit of any one of items 37 to 42, for use in the treatment of a disease in a mammal.
55. The α-subunit of human Interleukin 35 (SEQ ID NO: 1) and/or the β-subunit of human Interleukin 35 (SEQ ID NO: 2) for use of item 54, wherein the disease is an infectious disease, an autoimmune disease, cancer, preferably breast cancer, pancreatic cancer or lung cancer, a transplantation-related disease, preferably Graft-versus-Host-disease, an inflammatory disease, preferably a chronic inflammatory disease or an acute inflammatory disease, more preferably a chronic inflammatory bowel disease, sepsis, septic shock, diabetes or an allergic disease, preferably asthma.
56. The α-subunit of human Interleukin 35 (SEQ ID NO: 1) and/or the β-subunit of human Interleukin 35 (SEQ ID NO: 2) for use of item 54 or item 55, wherein the mammal is a human.
57. A method of treating an Interleukin 35-mediated disease, comprising the step of administering a composition comprising the α-subunit and/or the β-subunit of human Interleukin 35 (SEQ ID NOs: 1 and 2), preferably the subunit of any one of items 37 to 42.
58. The method of treating according to item 57, wherein the Interleukin 35-mediated disease is an infectious disease, an autoimmune disease, cancer, preferably breast cancer, pancreatic cancer or lung cancer, a transplantation-related disease, preferably Graft-versus-Host-disease, an inflammatory disease, preferably a chronic inflammatory disease or an acute inflammatory disease, more preferably a chronic inflammatory bowel disease, sepsis, septic shock, diabetes or an allergic disease, preferably asthma.
58. The use of the α-subunit of human Interleukin 35 (SEQ ID NO: 1) and/or the β-subunit of human Interleukin 35 (SEQ ID NO: 2), preferably the subunit of any one of items 37 to 42, for producing (a) binding reagent(s) against any of said subunit(s).
59. A method for producing (a) binding reagent(s) against the α-subunit of human Interleukin 35 (SEQ ID NO: 1) and/or the β-subunit of human Interleukin 35 (SEQ ID NO: 2), preferably against the subunit of any one of items 37 to 42.
60. The method according to item 59, comprising immunizing an animal with any of said subunits(s).
61. The method according to item 59 or item 60, comprising the in vitro generation of the binding reagent(s) against any of said subunit(s).
62. The method according to any one of items 59 to 61, comprising the selection of the binding reagent(s) specific for any of said subunit(s), preferably comprising the selection of the binding reagent(s) specific for any of said subunit(s) from a pool of binding reagent(s).
62. The method according to any one of items 59 to 61, wherein the binding reagent is selected from the group consisting of an antibody, a nanobody, a divalent antibody fragment, a monovalent antibody fragment, a proteinaceous binding molecule with antibody-like binding properties and an MHC molecule, preferably wherein the divalent antibody fragment is an (Fab)2′-fragment, or a divalent single-chain Fv fragment, and/or preferably wherein the monovalent antibody fragment is selected from the group consisting of a Fab fragment, a Fv fragment, and a single-chain Fv fragment (scFv) and/or preferably wherein the proteinaceous binding molecule with antibody-like binding properties is selected from the group consisting of an aptamer, a mutein based on a polypeptide of the lipocalin family, a glubody, a protein based on the ankyrin scaffold, a protein based on the crystalline scaffold, an adnectin, an avimer, a EGF-like domain, a Kringle-domain, a fibronectin type I domain, a fibronectin type II domain, a fibronectin type III domain, a PAN domain, a G1a domain, a SRCR domain, a Kunitz/Bovine pancreatic trypsin Inhibitor domain, tendamistat, a Kazal-type serine protease inhibitor domain, a Trefoil (P-type) domain, a von Willebrand factor type C domain, an Anaphylatoxin-like domain, a CUB domain, a thyroglobulin type I repeat, LDL-receptor class A domain, a Sushi domain, a Link domain, a Thrombospondin type I domain, an immunoglobulin domain or a an immunoglobulin-like domain, a C-type lectin domain, a MAM domain, a von Willebrand factor type A domain, a Somatomedin B domain, a WAP-type four disulfide core domain, a F5/8 type C domain, a Hemopexin domain, an SH2 domain, an SH3 domain, a Laminin-type EGF-like domain, a C2 domain, a nanobody, a adnectin, a tetranectin, a microbody, an affilin, an affibody or an ankyrin, a crystallin, a knottin, ubiquitin, a zinc-finger protein, an autofluorescent protein, an ankyrin or ankyrin repeat protein or a leucine-rich repeat protein, and an avimer.
63. A binding reagent which specifically binds to the α-subunit of human Interleukin 35 (SEQ ID NO: 1) and/or the β-subunit of human Interleukin 35 (SEQ ID NO: 2).
64. The binding reagent according to item 63, wherein the binding reagent is selected from the group consisting of an antibody, a nanobody, a divalent antibody fragment, a monovalent antibody fragment, a proteinaceous binding molecule with antibody-like binding properties and an MHC molecule, preferably wherein the divalent antibody fragment is an (Fab)2′-fragment, or a divalent single-chain Fv fragment, and/or preferably wherein the monovalent antibody fragment is selected from the group consisting of a Fab fragment, a Fv fragment, and a single-chain Fv fragment (scFv) and/or preferably wherein the proteinaceous binding molecule with antibody-like binding properties is selected from the group consisting of an aptamer, a mutein based on a polypeptide of the lipocalin family, a glubody, a protein based on the ankyrin scaffold, a protein based on the crystalline scaffold, an adnectin, an avimer, a EGF-like domain, a Kringle-domain, a fibronectin type I domain, a fibronectin type II domain, a fibronectin type III domain, a PAN domain, a G1a domain, a SRCR domain, a Kunitz/Bovine pancreatic trypsin Inhibitor domain, tendamistat, a Kazal-type serine protease inhibitor domain, a Trefoil (P-type) domain, a von Willebrand factor type C domain, an Anaphylatoxin-like domain, a CUB domain, a thyroglobulin type I repeat, LDL-receptor class A domain, a Sushi domain, a Link domain, a Thrombospondin type I domain, an immunoglobulin domain or an immunoglobulin-like domain, a C-type lectin domain, a MAM domain, a von Willebrand factor type A domain, a Somatomedin B domain, a WAP-type four disulfide core domain, a F5/8 type C domain, a Hemopexin domain, an SH2 domain, an SH3 domain, a Laminin-type EGF-like domain, a C2 domain, a nanobody, a adnectin, a tetranectin, a microbody, an affilin, an affibody or an ankyrin, a crystallin, a knottin, ubiquitin, a zinc-finger protein, an autofluorescent protein, an ankyrin or ankyrin repeat protein or a leucine-rich repeat protein, and an avimer.

### REFERENCES

1 Brocker, C., Thompson, D., Matsumoto, A., Nebert, D. W. & Vasiliou, V. Evolutionary divergence and functions of the human interleukin (IL) gene family. Human Genomics 5, 30 (2010). https://doi.org:10.1186/1479-7364-5-1-30
2 Vignali, D. A. & Kuchroo, V. K. IL-12 family cytokines: immunological playmakers. Nature immunology 13, 722-728 (2012). https://doi.org:ni.2366 [pii]10.1038/ni.2366
3 Hildenbrand, K., Aschenbrenner, I., Franke, F. C., Devergne, O. & Feige, M. J. Biogenesis and engineering of interleukin 12 family cytokines. Trends Biochem Sci 47, 936-949 (2022). https://doi.org:10.1016/j.tibs.2022.05.005
4 Tait Wojno, E. D., Hunter, C. A. & Stumhofer, J. S. The Immunobiology of the Interleukin-12 Family: Room for Discovery. Immunity 50, 851-870 (2019). https://doi.org:10.1016/j.immuni.2019.03.011
5 Yoshida, H. & Hunter, C. A. The immunobiology of interleukin-27. Annu Rev Immunol 33, 417-443 (2015). https://doi.org:10.1146/annurev-immunol-032414-112134
6 Ye, C., Yano, H., Workman, C. J. & Vignali, D. A. A. Interleukin-35: Structure, Function and Its Impact on Immune-Related Diseases. J Interferon Cytokine Res 41, 391-406 (2021). https://doi.org:10.1089/jir.2021.0147
7 Li, S. et al. STING-induced regulatory B cells compromise NK function in cancer immunity. Nature 610, 373-380 (2022). https://doi.org:10.1038/s41586-022-05254-3
8 Collison, L. W. et al. The inhibitory cytokine IL-35 contributes to regulatory T-cell function. Nature 450, 566-569 (2007). https://doi.org:nature06306 [pii] 10.1038/nature06306
9 Li, W., Gao, R., Xin, T. & Gao, P. Different expression levels of interleukin-35 in asthma phenotypes. Respir Res 21, 89 (2020). https://doi.org:10.1186/s12931-020-01356-6
10 Cheng, S. T. et al. Interleukin-35 Level Is Elevated in Patients with Chronic Hepatitis B Virus Infection. Int J Med Sci 15, 188-194 (2018). https://doi.org:10.7150/ijms.21957
11 Collison, L. W. et al. The inhibitory cytokine IL-35 contributes to regulatory T-cell function. Nature 450, 566-569 (2007). https://doi.org:10.1038/nature06306
12 Wirtz, S., Billmeier, U., McHedlidze, T., Blumberg, R. S. & Neurath, M. F. Interleukin-35 mediates mucosal immune responses that protect against T-cell-dependent colitis. Gastroenterology 141, 1875-1886 (2011). https://doi.org:10.1053/j.gastro.2011.07.040
13 Wang, Z. et al. Tumor-Derived IL-35 Promotes Tumor Growth by Enhancing Myeloid Cell Accumulation and Angiogenesis. The Journal of Immunology 190, 2415-2423 (2013). https://doi.org:10.4049/jimmunol. 1202535
14 Zhao, Z. et al. Increased interleukin-35 expression in tumor-infiltrating lymphocytes correlates with poor prognosis in patients with breast cancer. Cytokine 89, 76-81 (2017). https://doi.org:10.1016/j.cyto.2016.09.012
15 Chen, G. et al. Circulating low IL-23: IL-35 cytokine ratio promotes progression associated with poor prognosisin breast cancer. Am J Transl Res 8, 2255-2264 (2016).
16 Jin, P. et al. Circulating IL-35 in pancreatic ductal adenocarcinoma patients. Hum Immunol 75, 29-33 (2014). https://doi.org:10.1016/j.humimm.2013.09.018
17 Nicholl, M. B. et al. IL-35 promotes pancreas cancer growth through enhancement of proliferation and inhibition of apoptosis: evidence for a role as an autocrine growth factor. Cytokine 70, 126-133 (2014). https://doi.org:10.1016/i.cyto.2014.06.020
18 Huang, C. et al. Tumour-derived Interleukin 35 promotes pancreatic ductal adenocarcinoma cell extravasation and metastasis by inducing ICAM1 expression. Nature Communications 8, 14035 (2017). https://doi.org:10.1038/ncomms14035
19 Heim, L. et al. Increased expression of the immunosuppressive interleukin-35 in patients with non-small cell lung cancer. British journal of cancer 120, 903-912 (2019). https://doi.org:10.1038/s41416-019-0444-3
20 Yoon, C. et al. Charged residues dominate a unique interlocking topography in the heterodimeric cytokine interleukin-12. EMBO J 19, 3530-3541 (2000). https://doi.org:10.1093/emboj/19.14.3530
21 Lupardus, P. J. & Garcia, K. C. The structure of interleukin-23 reveals the molecular basis of p40 subunit sharing with interleukin-12. J Mol Biol 382, 931-941 (2008). https://doi.org: S0022-2836 (08) 00905-4 [pii] 10.1016/j.jmb.2008.07.051
22 Muller, S. I., Aschenbrenner, I., Zacharias, M. & Feige, M. J. An Interspecies Analysis Reveals Molecular Construction Principles of Interleukin 27. J Mol Biol 431, 2383-2393 (2019). https://doi.org:10.1016/j.jmb.2019.04.032
23 Rousseau, F. et al. IL-27 structural analysis demonstrates similarities with ciliary neurotrophic factor (CNTF) and leads to the identification of antagonistic variants. Proceedings of the National Academy of Sciences of the United States of America 107, 19420-19425 (2010). https://doi.org:1005793107 [pii] 10.1073/pnas. 1005793107
24 Glassman, C. R. et al. Structural basis for IL-12 and IL-23 receptor sharing reveals a gateway for shaping actions on T versus NK cells. Cell 184, 983-999.e924 (2021). https://doi.org:10.1016/j.cell.2021.01.018
25 Bloch, Y. et al. Structural Activation of Pro-inflammatory Human Cytokine IL-23 by Cognate IL-23 Receptor Enables Recruitment of the Shared Receptor IL-12Rbeta1. Immunity (2017). https://doi.org:10.1016/j.immuni.2017.12.008
26 Caveney, N. A., Glassman, C. R., Jude, K. M., Tsutsumi, N. & Garcia, K. C. Structure of the IL-27 quaternary receptor signaling complex. eLife 11 (2022). https://doi.org:10.7554/eLife.78463
27 Składanowska, K. et al. Structural basis of activation and antagonism of receptor signaling mediated by interleukin-27. Cell Rep 41, 111490 (2022). https://doi.org:10.1016/j.celrep.2022.111490
28 Jin, Y. et al. Structural insights into the assembly and activation of the IL-27 signaling complex. EMBO Rep 23 e55450 (2022). https://doi.org:10.15252/embr.202255450
29 Jones, L. L., Chaturvedi, V., Uyttenhove, C., Van Snick, J. & Vignali, D. A. Distinct subunit pairing criteria within the heterodimeric IL-12 cytokine family. Mol Immunol 51, 234-244 (2012). https://doi.org: S0161-5890 (12) 00209-X [pii] 10.1016/j.molimm.2012.03.025
30 Choi, J. K. et al. IL-12p35 Inhibits Neuroinflammation and Ameliorates Autoimmune Encephalomyelitis. Frontiers in immunology 8, 1258 (2017). https://doi.org:10.3389/fimmu.2017.01258
31 Dambuza, I. M. et al. IL-12p35 induces expansion of IL-10 and IL-35-expressing regulatory B cells and ameliorates autoimmune disease. Nat Commun 8, 719 (2017). https://doi.org:10.1038/s41467-017-00838-4
32 Chehboun, S. et al. Epstein-Barr virus-induced gene 3 (EBI3) can mediate IL-6 trans-signaling. J Biol Chem 292, 6644-6656 (2017). https://doi.org:10.1074/jbc.M116.762021
33 Wang, R. X. et al. Interleukin-35 induces regulatory B cells that suppress autoimmune disease. Nat Med 20, 633-641 (2014). https://doi.org:10.1038/nm.3554
34 Cassatella, M. A. et al. Human neutrophils activated by TLR8 agonists, with or without IFNγ, synthesize and release EBI3, but not IL-12, IL-27, IL-35, or IL-39. J Leukoc Biol (2020). https://doi.org:10.1002/jlb.3ma0520-054r
35 Collison, L. W. et al. The composition and signaling of the IL-35 receptor are unconventional. Nature immunology 13, 290-299 (2012). https://doi.org: ni.2227 [pii]10.1038/ni.2227
36 Devergne, O., Birkenbach, M. & Kieff, E. Epstein-Barr virus-induced gene 3 and the p35 subunit of interleukin 12 form a novel heterodimeric hematopoietin. Proceedings of the National Academy of Sciences of the United States of America 94, 12041-12046 (1997).
37 Gubler, U. et al. Coexpression of two distinct genes is required to generate secreted bioactive cytotoxic lymphocyte maturation factor. Proceedings of the National Academy of Sciences of the United States of America 88, 4143-4147 (1991).
38 Pflanz, S. et al. IL-27, a heterodimeric cytokine composed of EBI3 and p28 protein, induces proliferation of naive CD4 (+) T cells. Immunity 16, 779-790 (2002). https://doi.org: S1074761302003242 [pii]
39 Oppmann, B. et al. Novel p19 protein engages IL-12p40 to form a cytokine, IL-23, with biological activities similar as well as distinct from IL-12. Immunity 13, 715-725 (2000). https://doi.org: S1074-7613(00)00070-4 [pii]
40 Hildenbrand, K., Aschenbrenner, I., Franke, F. C., Devergne, O. & Feige, M. J. Biogenesis and engineering of interleukin 12 family cytokines. Trends Biochem Sci (2022). https://doi.org:10.1016/j.tibs.2022.05.005
41 Munro, S. & Pelham, H. R. A C-terminal signal prevents secretion of luminal ER proteins. Cell 48, 899-907 (1987). https://doi.org:10.1016/0092-8674(87)90086-9
42 Reitberger, S., Haimerl, P., Aschenbrenner, I., Esser-von Bieren, J. & Feige, M. J. Assembly-induced folding regulates interleukin 12 biogenesis and secretion. J Biol Chem 292, 8073-8081 (2017). https://doi.org:10.1074/jbc.M117.782284
43 Muller, S. I. et al. A folding switch regulates interleukin 27 biogenesis and secretion of its alpha-subunit as a cytokine. Proceedings of the National Academy of Sciences of the United States of America 116, 1585-1590 (2019). https://doi.org:10.1073/pnas. 1816698116
44 Bohnacker, S. et al. Influence of glycosylation on IL-12 family cytokine biogenesis and function. Mol Immunol 126, 120-128 (2020). https://doi.org:10.1016/j.molimm.2020.07.015
45 Devergne, O. et al. A novel interleukin-12 p40-related protein induced by latent Epstein-Barr virus infection in B lymphocytes. J Virol 70, 1143-1153 (1996).
46 Dong, J. et al. Amelioration of allergic airway inflammation in mice by regulatory IL-35 through dampening inflammatory dendritic cells. Allergy 70, 921-932 (2015). https://doi.org:10.1111/all. 12631
47 Schneider, C. et al. Induction of the nuclear receptor PPAR-γ by the cytokine GM-CSF is critical for the differentiation of fetal monocytes into alveolar macrophages. Nature immunology 15, 1026-1037 (2014). https://doi.org:10.1038/ni.3005
48 Yu, X. et al. The Cytokine TGF-β Promotes the Development and Homeostasis of Alveolar Macrophages. Immunity 47, 903-912.e904 (2017). https://doi.org:10.1016/j.immuni.2017.10.007
49 Liu, J., Guan, X. & Ma, X. Regulation of IL-27 p28 gene expression in macrophages through MyD88- and interferon-gamma-mediated pathways. J Exp Med 204, 141-152 (2007). https://doi.org:10.1084/jem.20061440
50 Chehimi, J. & Trinchieri, G. Interleukin-12: a bridge between innate resistance and adaptive immunity with a role in infection and acquired immunodeficiency. J Clin Immunol 14, 149-161 (1994). https://doi.org:10.1007/bf01533364
51 Mondal, S. et al. IL-12 p40 monomer is different from other IL-12 family members to selectively inhibit IL-12RB1 internalization and suppress EAE. Proceedings of the National Academy of Sciences of the United States of America 117, 21557-21567 (2020). https://doi.org:10.1073/pnas.2000653117
52 Collison, L. W. et al. IL-35-mediated induction of a potent regulatory T cell population. Nature immunology 11, 1093-1101 (2010). https://doi.org:10.1038/ni.1952
53 Shamji, M. H. et al. Role of IL-35 in sublingual allergen immunotherapy. J Allergy Clin Immunol 143, 1131-1142.e1134 (2019). https://doi.org:10.1016/j.jaci.2018.06.041
54 van der Veen, T. A., de Groot, L. E. S. & Melgert, B. N. The different faces of the macrophage in asthma. Curr Opin Pulm Med 26, 62-68 (2020). https://doi.org:10.1097/mcp.0000000000000647
55 Draijer, C., Robbe, P., Boorsma, C. E., Hylkema, M. N. & Melgert, B. N. Characterization of macrophage phenotypes in three murine models of house-dust-mite-induced asthma. Mediators Inflamm 2013, 632049 (2013). https://doi.org:10.1155/2013/632049
56 Chen, C. et al. Decreased concentration of IL-35 in plasma of patients with asthma and COPD. Asian Pac J Allergy Immunol 32, 211-217 (2014). https://doi.org:10.12932/ap0403.32.3.2014
57 Devergne, O., Coulomb-L′Hermine, A., Capel, F., Moussa, M. & Capron, F. Expression of Epstein-Barr virus-induced gene 3, an interleukin-12 p40-related molecule, throughout human pregnancy: involvement of syncytiotrophoblasts and extravillous trophoblasts. Am J Pathol 159, 1763-1776 (2001). https://doi.org:10.1016/S0002-9440(10)63023-4
58 Dokmeci, E. et al. EBI3 deficiency leads to diminished T helper type 1 and increased T helper type 2 mediated airway inflammation. Immunology 132, 559-566 (2011). https://doi.org:10.1111/j.1365-2567.2010.03401.x
59 Baran, P. et al. The balance of interleukin (IL)-6, IL-6⋅soluble IL-6 receptor (sIL-6R), and IL-6 sIL-6R.sgp130 complexes allows simultaneous classic and trans-signaling. J Biol Chem 293, 6762-6775 (2018). https://doi.org:10.1074/jbc.RA117.001163
60 Rose-John, S. The Soluble Interleukin 6 Receptor: Advanced Therapeutic Options in Inflammation. Clinical pharmacology and therapeutics 102, 591-598 (2017). https://doi.org:10.1002/cpt.782
61 Watanabe, A. et al. A Chaperone-Like Role for EBI3 in Collaboration With Calnexin Under Inflammatory Conditions. Frontiers in immunology 12, 757669 (2021). https://doi.org:10.3389/fimmu.2021.757669
62 Gerber, A. N., Abdi, K. & Singh, N. J. The subunits of IL-12, originating from two distinct cells, can functionally synergize to protect against pathogen dissemination in vivo. Cell Rep 37, 109816 (2021). https://doi.org:10.1016/j.celrep.2021.109816
63 Sullivan, J. A. et al. Treg-Cell-Derived IL-35-Coated Extracellular Vesicles Promote Infectious Tolerance. Cell Rep 30, 1039-1051.e1035 (2020). https://doi.org:10.1016/j.celrep.2019.12.081
64 Aparicio-Siegmund, S. et al. Recombinant p35 from bacteria can form (IL-)12, but Not IL-35. PLoS One 9, e107990 (2014). https://doi.org:10.1371/journal.pone.0107990
65 Meier, S. et al. The molecular basis of chaperone-mediated interleukin 23 assembly control. Nat Commun 10, 4121 (2019). https://doi.org:10.1038/s41467-019-12006-x
66 Dam, J., Velikovsky, C. A., Mariuzza, R. A., Urbanke, C. & Schuck, P. Sedimentation velocity analysis of heterogeneous protein-protein interactions: Lamm equation modeling and sedimentation coefficient distributions c(s). Biophys J 89, 619-634 (2005). https://doi.org:10.1529/biophysj.105.059568
67 Dietz, K. et al. Age dictates a steroid-resistant cascade of Wnt5a, transglutaminase 2, and leukotrienes in inflamed airways. J Allergy Clin Immunol 139, 1343-1354.e1346 (2017). https://doi.org:10.1016/j.jaci.2016.07.014
68 Esser-von Bieren, J. et al. Antibodies trap tissue migrating helminth larvae and prevent tissue damage by driving IL-4Ra-independent alternative differentiation of macrophages. PLoS Pathog 9, e1003771 (2013). https://doi.org:10.1371/journal.ppat.1003771
69 Prodjinotho, U. F. et al. Helminthic dehydrogenase drives PGE (2) and IL-10 production in monocytes to potentiate Treg induction. EMBO Rep 23, e54096 (2022). https://doi.org:10.15252/embr.202154096
70 Layland, L. E. et al. Pronounced phenotype in activated regulatory T cells during a chronic helminth infection. Journal of immunology 184, 713-724 (2010). https://doi.org:10.4049/jimmunol.0901435
71 Jumper, J. et al. Highly accurate protein structure prediction with AlphaFold. Nature 596, 583-589 (2021). https://doi.org:10.1038/s41586-021-03819-2
72 Evans, R. et al. Protein complex prediction with AlphaFold-Multimer. bioRxiv, 2021.2010.2004.463034 (2022). https://doi.org:10.1101/2021.10.04.463034

1. A method for producing the α-subunit of human Interleukin 35 (SEQ ID NO: 1) from human Interleukin 12 and/or the β-subunit EBI3 of human Interleukin 35 (SEQ ID NO: 2) from human Interleukin 27, comprising the steps of:
(a) introducing into a nucleic acid molecule encoding the recombinant α- and β-subunits of the human Interleukin 12 polypeptide (SEQ ID NOs: 3 and 4) a nucleotide sequence mutating at least one amino acid residue of the α-subunit of human Interleukin 12 (SEQ ID NO: 3) and/or mutating at least one amino acid residue of the β-subunit of human Interleukin 12 (SEQ ID NO: 4), and/or
(b) introducing into a nucleic acid molecule encoding the recombinant α- and β-subunits of the human Interleukin 27 polypeptide (SEQ ID NOs: 5 and 6) a nucleotide sequence mutating at least one amino acid residue of the α-subunit of human Interleukin 27 (SEQ ID NO: 5), and
(c) introducing the obtained nucleic acid molecule(s) of steps (a) and/or (b) for expression into a suitable host cell or into a suitable cell extract or cell lysate.

2. The method according to claim 1, wherein the method further comprises a step (d) comprising the isolation of human Interleukin 12 and/or the isolation of human Interleukin 27 from the product of step (c), preferably wherein the isolation of human Interleukin 12 and/or the isolation of human Interleukin 27 is/are carried out by affinity chromatography.

3. The method according to claim 2, wherein the method further comprises a step (e) comprising the separation of the α-subunit of human Interleukin 12 (SEQ ID NO: 12) from the isolated human Interleukin 12 of step (d) and/or the separation of the β-subunit of human Interleukin 27 (SEQ ID NO: 6) from the isolated human Interleukin 27 of step (d), optionally wherein the separation of the α-subunit of human Interleukin 12 (SEQ ID NO: 3) from the isolated human Interleukin 12 of step (d) and/or the separation of the β-subunit of human Interleukin 27 (SEQ ID NO: 6) from the isolated human Interleukin 27 of step (d) is carried out by denaturation and subsequent renaturation under physiological conditions.

4. The method according to claim 3, wherein the method further comprises a step (f) comprising the purification of the α-subunit of human Interleukin 12 (SEQ ID NO: 3) of step (e) and/or the purification of the β-subunit of human Interleukin 27 (SEQ ID NO: 6) of step (e), optionally wherein the purification of the α-subunit of human Interleukin 12 (SEQ ID NO: 3) of step (e) and/or the purification of the β-subunit of human Interleukin 27 (SEQ ID NO: 6) of step (e) is/are carried out by size exclusion chromatography.

5. The method according to any one of the preceding claims, wherein step (a) comprises introducing into a nucleic acid molecule encoding the recombinant α- and β-subunits of the human Interleukin 12 polypeptide (SEQ ID NOs: 3 and 4) a nucleotide sequence mutating the amino acid residue of the α-subunit of human Interleukin 12 (SEQ ID NO: 3) at sequence position 96, preferably wherein step (a) comprises introducing into a nucleic acid molecule encoding the recombinant α- and β-subunits of the human Interleukin 12 polypeptide (SEQ ID NOs: 3 and 4) a nucleotide sequence mutating the amino acid residue of the α-subunit of human Interleukin 12 (SEQ ID NO: 3) at sequence position 96 to any other amino acid than cysteine, preferably to serine or alanine, more preferably to serine, preferably wherein the nucleotide sequence further comprises a nucleotide sequence encoding an affinity tag, and/or
wherein step (a) comprises introducing into a nucleic acid molecule encoding the recombinant α- and β-subunits of the human Interleukin 12 polypeptide (SEQ ID NOs: 3 and 4) a nucleotide sequence mutating the amino acid residue of the β-subunit of human Interleukin 12 (SEQ ID NO: 4) at sequence position 199, preferably wherein step (a) comprises introducing into a nucleic acid molecule encoding the recombinant α- and β-subunits of the human Interleukin 12 polypeptide (SEQ ID NOs: 3 and 4) a nucleotide sequence mutating the amino acid residue of the β-subunit of human Interleukin 12 (SQ ID NO: 4) at sequence position 199 to serine.

6. The method according to any one of the preceding claims, wherein step (a) comprises introducing into a nucleic acid molecule encoding the recombinant α- and β-subunits of the human Interleukin 12 polypeptide (SEQ ID NOs: 3 and 4) a nucleotide sequence mutating the amino acid residue of the α-subunit of human Interleukin 12 (SEQ ID NO: 3) at sequence position 96 and introducing into a nucleic acid molecule encoding the recombinant α- and β-subunits of the human Interleukin 12 polypeptide (SEQ ID NOs: 3 and 4) a nucleotide sequence mutating the amino acid residue of the β-subunit of human Interleukin 12 (SEQ ID NO: 4) at sequence position 199, preferably wherein step (a) comprises introducing into a nucleic acid molecule encoding the recombinant α- and β-subunits of the human Interleukin 12 polypeptide (SEQ ID NOs: 3 and 4) a nucleotide sequence mutating the amino acid residue of the α-subunit of human Interleukin 12 (SEQ ID NO: 3) at sequence position 96 to any other amino acid than cysteine, preferably to serine or alanine, more preferably to serine, and introducing into a nucleic acid molecule encoding the recombinant α- and β-subunits of the human Interleukin 12 polypeptide (SEQ ID NOs: 3 and 4) a nucleotide sequence mutating the amino acid residue of the β-subunit of human Interleukin 12 (SEQ ID NO: 4) at sequence position 199 to any other amino acid than cysteine, preferably to serine, and/or
wherein step (b) comprises introducing into a nucleic acid molecule encoding the recombinant α- and β-subunits of the human Interleukin 27 polypeptide (SEQ ID NOs: 5 and 6) a nucleotide sequence mutating the amino acid residue of the α-subunit of human Interleukin 27 (SEQ ID NO: 5) at sequence position 162, preferably wherein step (b) comprises introducing into a nucleic acid molecule encoding the recombinant α- and β-subunits of the human Interleukin 27 polypeptide (SEQ ID NOs: 5 and 6) a nucleotide sequence mutating the amino acid residue of the α-subunit of human Interleukin 27 (SEQ ID NO: 5) at sequence position 162 to cysteine, preferably wherein the nucleotide sequence further comprises a nucleotide sequence encoding an affinity tag.

7. The method according to any one of the preceding claims, wherein steps (a) and (b) are carried out simultaneously or step (a) is carried out before step (b) or step (b) is carried out before step (a).

8. The method according to any one of the preceding claims, wherein step (a) comprises introducing into a nucleic acid molecule encoding the recombinant α- and β-subunits of the human Interleukin 12 polypeptide (SEQ ID NOs: 3 and 4) a nucleotide sequence mutating the amino acid residue of the α-subunit of human Interleukin 12 (SEQ ID NO: 3) at sequence position 96, and wherein step (a) comprises introducing into a nucleic acid molecule encoding the recombinant α- and β-subunits of the human Interleukin 12 polypeptide (SEQ ID NOs: 3 and 4) a nucleotide sequence mutating the amino acid residue of the β-subunit of human Interleukin 12 (SEQ ID NO: 4) at sequence position 199, and wherein step (b) comprises introducing into a nucleic acid molecule encoding the recombinant α- and β-subunits of the human Interleukin 27 polypeptide (SEQ ID NOs: 5 and 6) a nucleotide sequence mutating the amino acid residue of the α-subunit of human Interleukin 27 (SEQ ID NO: 5) at sequence position 162,
preferably wherein step (a) comprises introducing into a nucleic acid molecule encoding the recombinant α- and β-subunits of the human Interleukin 12 polypeptide (SEQ ID NOs: 3 and 4) a nucleotide sequence mutating the amino acid residue of the α-subunit of human Interleukin 12 (SEQ ID NO: 3) at sequence position 96 to any other amino acid than cysteine, preferably to serine or alanine, more preferably to serine, and wherein step (a) comprises introducing into a nucleic acid molecule encoding the recombinant α- and β-subunits of the human Interleukin 12 polypeptide (SEQ ID NOs: 3 and 4) a nucleotide sequence mutating the amino acid residue of the β-subunit of human Interleukin 12 (SEQ ID NO: 4) at sequence position 199 to any other amino acid than cysteine, preferably to serine or alanine, more preferably to serine, and wherein step (b) comprises introducing into a nucleic acid molecule encoding the recombinant α- and β-subunits of the human Interleukin 27 polypeptide (SEQ ID NOs: 5 and 6) a nucleotide sequence mutating the amino acid residue of the α-subunit of human Interleukin 27 (SEQ ID NO: 5) at sequence position 162 to cysteine.

9. The α-subunit of human Interleukin 12 (SEQ ID NO: 3) obtained according to the method of any one of claims 1 to 8, preferably wherein said α-subunit of human Interleukin 12 (SEQ ID NO: 3) is identical to the α-subunit of human Interleukin 35 (SEQ ID NO: 1), and/or wherein the amino acid residue at amino acid position 96 is mutated, preferably wherein the amino acid residue at amino acid position 96 is mutated to any other amino acid than cysteine, more preferably to serine or alanine, even more preferably to serine.

10. The β-subunit of human Interleukin 27 (SEQ ID NO: 6) obtained according to the method of any one of claims 1 to 8, preferably wherein said β-subunit of human Interleukin 27 (SEQ ID NO: 6) is identical to the β-subunit of human Interleukin 35 (SEQ ID NO: 2).

11. A nucleic acid molecule comprising a nucleotide sequence encoding the α-subunit of human Interleukin 12 (SEQ ID NO: 3) according to claim 9 or a nucleic acid molecule comprising a nucleotide sequence encoding the β-subunit of human Interleukin 27 (SEQ ID NO: 6) according to claim 10, preferably wherein the nucleic acid molecule is operably linked to a regulatory sequence to allow expression of the nucleic acid molecule, more preferably wherein the regulatory sequence comprises a promoter sequence, or said nucleic acid molecule being comprised in a vector, or a host cell containing said nucleic acid molecule, or an immune modulator comprising a subunit of claim 9 or claim 10.

12. Use of the α-subunit of human Interleukin 35 (SEQ ID NO: 1) and/or of the β-subunit of human Interleukin 35 (SEQ ID NO: 2), preferably of a subunit of claim 9 or 10, for the manufacture of a medicament for treating a disease in a mammal, preferably wherein the mammal is a human, preferably wherein the disease is an infectious disease, an autoimmune disease, cancer, more preferably breast cancer, pancreatic cancer or lung cancer, a transplantation-related disease, more preferably Graft-versus-Host-disease, an inflammatory disease, more preferably a chronic inflammatory disease or an acute inflammatory disease, even more preferably a chronic inflammatory bowel disease, sepsis, septic shock, diabetes or an allergic disease, more preferably asthma in a mammal.

13. The α-subunit of human Interleukin 35 (SEQ ID NO: 1) and/or the β-subunit of human Interleukin 35 (SEQ ID NO: 2), preferably the subunit of claim 9 or claim 10, for use as a medicament or a drug, or the α-subunit of human Interleukin 35 (SEQ ID NO: 1) and/or the β-subunit of human Interleukin 35 (SEQ ID NO: 2), preferably the subunit of claim 9 or claim 10, for use in the treatment of a disease in a mammal, preferably wherein the disease is an infectious disease, an autoimmune disease, cancer, more preferably breast cancer, pancreatic cancer or lung cancer, a transplantation-related disease, more preferably Graft-versus-Host-disease, an inflammatory disease, more preferably a chronic inflammatory disease or an acute inflammatory disease, even more preferably a chronic inflammatory bowel disease, sepsis, septic shock, diabetes or an allergic disease, more preferably asthma, preferably wherein the mammal is a human.

14. A method of treating an Interleukin 35-mediated disease, comprising the step of administering a composition comprising the α-subunit and/or the β-subunit of human Interleukin 35 (SEQ ID NOs: 1 and 2), preferably the subunit of claim 9 or claim 10, preferably wherein the Interleukin 35-mediated disease is an infectious disease, an autoimmune disease, cancer, more preferably breast cancer, pancreatic cancer or lung cancer, a transplantation-related disease, more preferably Graft-versus-Host-disease, an inflammatory disease, more preferably a chronic inflammatory disease or an acute inflammatory disease, even more preferably a chronic inflammatory bowel disease, sepsis, septic shock, diabetes or an allergic disease, more preferably asthma.

15. The use of the α-subunit of human Interleukin 35 (SEQ ID NO: 1) and/or the β-subunit of human Interleukin 35 (SEQ ID NO: 2), preferably the subunit of claim 9 or claim 10, for producing a binding reagent against any of said subunit(s), or
a method for producing a binding reagent against the α-subunit of human Interleukin 35 (SEQ ID NO: 1) and/or the β-subunit of human Interleukin 35 (SEQ ID NO: 2), preferably against the subunit of claim 9 or claim 10, preferably wherein the method comprises immunizing an animal with any of said subunit(s), and/or preferably wherein the method comprises the in vitro generation of the binding reagent(s) against any of said subunit(s), and/or preferably wherein the method comprises the selection of the binding reagents(s) specific for any of said subunit(s), more preferably comprising the selection of the binding reagents(s) specific for any of said subunit(s) from a pool of binding reagents(s), or a binding reagent which specifically binds to the α-subunit of human Interleukin 35 (SEQ ID NO: 1) and/or the β-subunit of human Interleukin 35 (SEQ ID NO: 2), preferably wherein the binding reagent is selected from the group consisting of an antibody, a nanobody, a divalent antibody fragment, a monovalent antibody fragment, a proteinaceous binding molecule with antibody-like binding properties and an MHC molecule, more preferably wherein the divalent antibody fragment is an (Fab)2′-fragment, or a divalent single-chain Fv fragment, and/or more preferably wherein the monovalent antibody fragment is selected from the group consisting of a Fab fragment, a Fv fragment, and a single-chain Fv fragment (scFv) and/or more preferably wherein the proteinaceous binding molecule with antibody-like binding properties is selected from the group consisting of an aptamer, a mutein based on a polypeptide of the lipocalin family, a glubody, a protein based on the ankyrin scaffold, a protein based on the crystalline scaffold, an adnectin, an avimer, a EGF-like domain, a Kringle-domain, a fibronectin type I domain, a fibronectin type II domain, a fibronectin type III domain, a PAN domain, a G1a domain, a SRCR domain, a Kunitz/Bovine pancreatic trypsin Inhibitor domain, tendamistat, a Kazal-type serine protease inhibitor domain, a Trefoil (P-type) domain, a von Willebrand factor type C domain, an Anaphylatoxin-like domain, a CUB domain, a thyroglobulin type I repeat, LDL-receptor class A domain, a Sushi domain, a Link domain, a Thrombospondin type I domain, an immunoglobulin domain or an immunoglobulin-like domain, a C-type lectin domain, a MAM domain, a von Willebrand factor type A domain, a Somatomedin B domain, a WAP-type four disulfide core domain, a F5/8 type C domain, a Hemopexin domain, an SH2 domain, an SH3 domain, a Laminin-type EGF-like domain, a C2 domain, a nanobody, a adnectin, a tetranectin, a microbody, an affilin, an affibody or an ankyrin, a crystallin, a knottin, ubiquitin, a zinc-finger protein, an autofluorescent protein, an ankyrin or ankyrin repeat protein or a leucine-rich repeat protein, and an avimer.


SEQUENCE LISTING (35 sequences)

SEQ ID NO: 1 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 2 - protein, 229 aa, Homo sapiens
MTPQLLLALVLWASCPPCSGRKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 3 - protein, 219 aa, Homo sapiens; identical to SEQ ID NO: 1

SEQ ID NO: 4 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 5 - protein, 243 aa, Homo sapiens
MGQTAGDLGWRLSLLLLPLLLVQAGVWGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSALQGPAQVSWPQLLSTYRLLHSLELVLSRAVRELLLLSKAGHSVWPLGFPTLSPQP

SEQ ID NO: 6 - protein, 229 aa, Homo sapiens; identical to SEQ ID NO: 2

SEQ ID NO: 7 - protein, 219 aa, synthetic construct
  REGION 1..219: note = C96S mutant of alpha-subunit of IL-12
MCPARSLLLVATLVLLDHLSLARNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 8 - protein, 328 aa, synthetic construct
  REGION 1..328: note = C199S mutant of beta-subunit of IL-12
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSASPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 9 - protein, 243 aa, synthetic construct
  REGION 1..243: note = L162C mutant of alpha-subunit of IL-27
MGQTAGDLGWRLSLLLLPLLLVQAGVWGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNCPEEEEEEEEEEEEERKGLLPGALGSALQGPAQVSWPQLLSTYRLLHSLELVLSRAVRELLLLSKAGHSVWPLGFPTLSPQP

SEQ ID NO: 10 - protein, 20 aa, synthetic construct
  REGION 1..20: note = 18S primer forward sequence
GTAACCCGTTGAACCCCATT

SEQ ID NO: 11 - protein, 20 aa, synthetic construct
  REGION 1..20: note = 18S primer reverse sequence
CCATCCATTCGGTAGTAGCG

SEQ ID NO: 12 - protein, 20 aa, synthetic construct
  REGION 1..20: note = ACTB primer forward sequence
GGATGCAGAAGGAGATCACT

SEQ ID NO: 13 - protein, 20 aa, synthetic construct
  REGION 1..20: note = ACTB primer reverse sequence
CGATCCACACGGAGTACTTG

SEQ ID NO: 14 - protein, 22 aa, synthetic construct
  REGION 1..22: note = CXCL8 primer forward sequence
GAAGTTTTTGAAGAGGGCTGAG

SEQ ID NO: 15 - protein, 21 aa, synthetic construct
  REGION 1..21: note = CXCL8 primer reverse sequence
TGCTTGAAGTTTCACTGGCAT

SEQ ID NO: 16 - protein, 18 aa, synthetic construct
  REGION 1..18: note = GAPDH primer forward sequence
GAAGGTGAAGGTCGGAGT

SEQ ID NO: 17 - protein, 20 aa, synthetic construct
  REGION 1..20: note = GAPDH primer reverse sequence
GAAGATGGTGATGGGATTTC

SEQ ID NO: 18 - protein, 20 aa, synthetic construct
  REGION 1..20: note = IL1B primer forward sequence
AGAAGTACCTGAGCTCGCCA

SEQ ID NO: 19 - protein, 22 aa, synthetic construct
  REGION 1..22: note = IL1B primer reverse sequence
CTGGAAGGAGCACTTCATCTGT

SEQ ID NO: 20 - protein, 21 aa, synthetic construct
  REGION 1..21: note = IL6 primer forward sequence
ACATGTGTGAAAGCAGCAAAG

SEQ ID NO: 21 - protein, 22 aa, synthetic construct
  REGION 1..22: note = IL6 primer reverse sequence
GGCAAGTCTCCTCATTGAATCC

SEQ ID NO: 22 - protein, 21 aa, synthetic construct
  REGION 1..21: note = TNF primer forward sequence
CCCATGTTGTAGCAAACCCTC

SEQ ID NO: 23 - protein, 20 aa, synthetic construct
  REGION 1..20: note = TNF primer reverse sequence
TATCTCTCAGCTCCACGCCA

SEQ ID NO: 24 - protein, 20 aa, synthetic construct
  REGION 1..20: note = TGFB primer forward sequence
CACGCAGTACAGCAAGGTCC

SEQ ID NO: 25 - protein, 20 aa, synthetic construct
  REGION 1..20: note = TGFB primer reverse sequence
CCACGTAGTACACGATGGGC

SEQ ID NO: 26 - protein, 20 aa, synthetic construct
  REGION 1..20: note = IL5 primer forward sequence
TCTCCAGTGTGCCTATTCCC

SEQ ID NO: 27 - protein, 20 aa, synthetic construct
  REGION 1..20: note = IL5 primer reverse sequence
CGAACTCTGCTGATAGCCAA

SEQ ID NO: 28 - protein, 229 aa, synthetic construct
  REGION 1..229: note = alpha-subunit of IL-12 with KDEL-sequence
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGSGSKDEL

SEQ ID NO: 29 - protein, 239 aa, synthetic construct
  REGION 1..239: note = EBI3 with KDEL-sequence
MTPQLLLALVLWASCPPCSGRKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGSGSGSKDEL

SEQ ID NO: 30 - protein, 338 aa, synthetic construct
  REGION 1..338: note = C199S-mutant of beta-subunit of IL-12 with KDEL-sequence
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSASPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGSGSGSKDEL

SEQ ID NO: 31 - protein, 252 aa, synthetic construct
  REGION 1..252: note = alpha-subunit of IL-27 with KDEL-sequence
MGQTAGDLGWRLSLLLLPLLLVQAGVWGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSALQGPAQVSWPQLLSTYRLLHSLELVLSRAVRELLLLSKAGHSVWPLGFPTLSPQPGSGSGSKDE

SEQ ID NO: 32 - protein, 234 aa, synthetic construct
  REGION 1..234: note = C96S-mutant of alpha-subunit of IL-12 with His-sequence
MCPARSLLLVATLVLLDHLSLARNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGENLYFQSHHHHHH

SEQ ID NO: 33 - protein, 216 aa, synthetic construct
  REGION 1..216: note = alpha-subunit of mouse IL-27 with His-sequence
FPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDSHHHHHHHHHH

SEQ ID NO: 34 - protein, 263 aa, synthetic construct
  REGION 1..263: note = L162C-mutant of alpha-subunit of IL-27 with His-sequence
MGQTAGDLGWRLSLLLLPLLLVQAGVWGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNCPEEEEEEEEEEEEERKGLLPGALGSALQGPAQVSWPQLLSTYRLLHSLELVLSRAVRELLLLSKAGHSVWPLGFPTLSPQPGGLEVLFQGPGGGGHHHHHH

SEQ ID NO: 35 - protein, 345 aa, synthetic construct
  REGION 1..345: note = C199S-mutant of beta-subunit of IL-12 with His-sequence
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSASPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGSENLYFQSHHHHHHGS